(19) United States
(12) Patent Application Publication
(10) Pub. No.: US 2022/0128543 A1
(43) Pub. Date: Apr. 28, 2022

(54) MACROPHAGE MARKERS IN CANCER

(71) Applicant: The University Court of the University of Edinburgh, Edinburgh (GB)

(72) Inventors: Jeff Pollard, Edinburgh (GB); Luca Cassetta, Edinburgh (GB); Stamatina Fragkogianni, Edinburgh (GB)

(21) Appl. No.: 17/436,163

(22) PCT Filed: Mar. 6, 2020
(86) PCT No.: PCT/EP2020/056126

§ 371 Date: Sep. 3, 2021

(30) Foreign Application Priority Data
Mar. 6, 2019 (GB) 1903014.7

(51) Int. Cl. G01N 33/50; C12Q 1/6886; G01N 33/574; G01N 33/53; C12Q 1/6809

(52) U.S. Cl. CPC G01N 33/5023; C12Q 1/6886; G01N 33/57488; C12Q 2600/118; C12Q 1/6809; C12Q 2600/158; G01N 33/5302

(57) ABSTRACT

The present invention relates to the diagnosis, prognosis, prediction and/or treatment of cancer, for example breast cancer, lung cancer or colon cancer. The present invention provides methods of diagnosing and/or prognosing cancer, predicting efficacy of treatment for cancer, assessing outcome of treatment for cancer, assessing likelihood of metastasis and/or assessing recurrence of cancer, the methods comprising a) analyzing a biological sample obtained from a subject to determine the presence of one or more target molecules representative of expression of SIGLEC1 and/or CCL8; and b) comparing the expression level of SIGLEC1 and/or CCL8 determined in (a) with one or more reference values, wherein whether there is a difference in the expression of SIGLEC1 and/or CCL8 in the sample from the subject or not compared to the one or more reference values is indicative of a clinical indication. The invention further provides related methods for treatment of cancer, kits and assay devices for use in the methods, and methods for identifying molecules for use in treating cancer.

### FIELD OF THE INVENTION

The present invention relates to methods for the diagnosis, prognosis, prediction and/or treatment of cancer, for example breast cancer, lung cancer or colon cancer. The invention also concerns kits and assay devices for use in the methods of the invention. Further, the invention concerns methods of finding new therapeutic molecules for treating cancer.

### BACKGROUND TO THE INVENTION

The tumor microenvironment is a dominant player in the regulation of tumor progression and growth. Tumors not only comprise of malignant cells but also a complex stroma in which immune cells are highly represented; cancer cells acquire the ability to “distract and educate” the immune system so that their abnormal proliferation is not detected, but rather promoted.

In mouse models of cancer, tumor associated macrophages (TAMs) promote progression to malignancy. Experimental models have indicated that Tumor Associated Macrophages (TAMs), whose density correlates with poor prognosis in many human cancers, promote angiogenesis, blunt anti-tumor cytolytic T cell responses, enhance tumor extravasation, dissemination and overt metastasis [1]. TAMs are largely but not wholly derived from circulating monocytes recruited by neoplastic cells and then educated to differentiate into TAMs. In humans however, little is known regarding these activities and the role of TAMs in cancer.

Breast Cancer is the most common cancer in women. Early detection of tumors significantly improves survival rates; more than 90% of women diagnosed with early stage breast cancer survive for at least five years. Consequently mammographic screening, by enabling early detection, reduces mortality in women 50-74 years of age although efficacy is more limited for younger women, with false positive resulting in overdiagnosis and potentially unnecessary treatment. Other early detection screening methods (e.g., MRI, ultrasonography, clinical and self-breast examination) are inadequate at present to reduce breast cancer mortality. These data underlie an urgent need for improved detection and clinical management of malignant cancers.

SIGLEC1 (CD169) is a sialic-acid binding lectin mainly expressed by macrophages in the lymph-node and in the spleen [2]; marginal zone CD169+ macrophages are reported to recruit regulatory T cells (Tregs) and Dendritic cells through CCL22 after exposure with apoptotic cells, contributing to the establishment of the immunological tolerance in the spleen. In the lymph node CD169⁺ macrophages can capture exosomes to decrease the probability of self-antigen response [2].

CCL8 (C-C motif chemokine ligand 8) encodes a cytokine that displays chemotactic activity for monocytes, lymphocytes, basophils and eosinophils. It is a member of the CC subfamily which is characterized by two adjacent cysteine residues.

### SUMMARY OF THE INVENTION

The present invention provides methods of diagnosing and/or prognosing cancer, predicting efficacy of treatment for cancer, assessing outcome of treatment for cancer, assessing likelihood of metastasis or assessing recurrence of cancer. The methods comprise the steps of a) analyzing a biological sample obtained from a subject to determine the presence of one or more target molecules representative of expression of SIGLEC1 and/or CCL8; and b) comparing the expression level of SIGLEC1 and/or CCL8 determined in (a) with one or more reference values, wherein whether there is a difference in the expression of SIGLEC1 and/or CCL8 in the sample from the subject or not compared to the one or more reference values is indicative of a clinical indication. Preferably the methods involve considering the expression levels of SIGLEC1.

Suitably said clinical indication comprises one or more of the presence or absence of cancer in the subject, likelihood of metastasis, likely outcome of treatment of the cancer in the subject, likelihood of recurrence of the cancer following treatment, an indication of whether the prognosis for the cancer treatment and subject is good or poor and/or predicted survival (life expectancy) of the subject, likelihood of benign tissues progressing to malignancy.

Preferably, the methods comprise analyzing a biological sample from the subject to determine the presence of target molecules representative of expression of SIGLEC1 and/or CCL8, and optionally CD163 and/or CD68, and comparing the determined expression level(s) with one or more reference values, wherein whether there is a difference in the expression of SIGLEC1, CCL8, CD163, and/or CD68 in the sample from the subject compared to the one or more reference values is indicative of a clinical indication. Thus in some embodiments of the methods of the invention, step a) may comprise analyzing a biological sample obtained from a subject to determine the presence of target molecules representative of expression of SIGLEC1, and one or more of CCL8, CD163, and CD68; and b) comparing the expression levels of SIGLEC1, and one or more of CCL8, CD163, andr CD68, determined in (a) with one or more reference values, wherein whether there is a difference in the expression of SIGLEC1, CCL8, CD163 and/or CD68 in the sample from the subject compared to the one or more reference values is indicative of a clinical indication. The analysis of the levels of CCL8 and/or CD163 and/or CD68 may be each carried out simultaneously, sequentially or separately from the analysis of the levels of SIGLEC1.

In some embodiments of the methods of the invention, step a) may comprise analyzing a biological sample obtained from a subject to determine the presence of target molecules representative of expression of CCL8, and one or more of SIGLEC1, CD163, and CD68; and b) comparing the expression levels of CCL8, and the one or more of SIGLEC1, CD163, and CD68, determined in (a) with one or more reference values, wherein whether there is a difference in the expression of CCL8, SIGLEC1, CD163 and/or CD68 in the sample from the subject compared to the one or more reference values is indicative of a clinical indication. The analysis of the levels of SIGLEC1 and/or CD163 and/or CD68 may be each carried out simultaneously, sequentially or separately from the analysis of the levels of CCL8.

The invention also provides associated methods of treating cancer in a subject. The methods comprise the steps of a) analyzing a biological sample obtained from a subject to determine the presence of one or more target molecules representative of expression of SIGLEC1 and/or CCL8; and b) comparing the expression level of SIGLEC1 and/or CCL8 determined in (a) with one or more reference values, and providing the subject with a particular treatment for cancer or not according to whether there is a difference in the expression of SIGLEC1 in the sample from the subject or not compared to the one or more reference values. As explained for the methods above, preferably the methods of treatment may comprise determining the expression levels of two or more of SIGLEC1, CCL8,CD163, and CD68, and comparing those expression levels with reference values. Thus providing the subject with a particular treatment for cancer or not may be according to whether there is a difference in the expression of SIGLEC1 and/or CCL8, and optionally CD163 and/or CD68, in the sample from the subject compared to the one or more reference values.

The invention also provides kits for use in the above methods, the kits comprise binding partners capable of binding to target molecules representative of expression of SIGLEC1 and/or CCL8, and optionally CD163 and/or CD68. Preferably the kits also comprise indicators capable of indicating when said binding occurs.

The invention also provides an assay device for use in embodiments of the above methods, the device comprising: a) a loading area for receipt of a biological sample; b) binding partners specific for target molecules representative of expression of SIGLEC1 and/or CCL8, and optionally CD163 and/or CD68; and c) detection means to detect the levels of said target molecules present in the sample. Preferably, the invention provides an assay device for use in embodiments of the above methods, the device comprising: a) a loading area for receipt of a biological sample; b) binding partners specific for target molecules representative of expression of CCL8, and optionally SIGLEC1 and/or CD163 and/or CD68; and c) detection means to detect the levels of said target molecules present in the sample.

The invention also provides a method of identifying one or more molecules for use in treating cancer. The method may comprise identifying a molecule that binds SIGLEC1, or CD163, or CCL8 or a CCL8 receptor. Thus the method may comprise the steps of a) preparing a candidate molecule, b) contacting a cell that expresses SIGLEC1, CD163, CCL8, and/or a CCL8 receptor, with the candidate molecule, and c) determining whether said candidate molecule binds the SIGLEC1, CD163, CCL8 and/or CCL8 receptor and affects its activity. Alternatively, the method may comprise identifying a molecule that interferes with expression of SIGLEC1, or CD163, or CCL8 or a CCL8 receptor. Thus, the method may comprise the steps of a) preparing a candidate molecule, b) contacting a cell that expresses SIGLEC1, CD163, CCL8, and/or a CCL8 receptor, with the candidate molecule, and c) determining whether said candidate molecule interferes with either transcription or translation of the SIGLEC1, CD163, CCL8 and/or CCL8 receptor and thereby affects its expression. A candidate molecule that inhibits the activity, and/or downregulates the expression, of the SIGLEC1, CD163, or CCL8 or a CCL8 receptor may be identified as for use in treating cancer.

Preferably the cell used in the method of identifying a molecule will be an induced Pluripotent stem cell (iPS) derived macrophage conditioned by tumor cell conditioned media, or the cell may be from a mouse model of cancer. Activities include the ability of a cell to immunosuppress an immune response through blocking action of cytotoxic cells such as T cells or NK cells, to block the migration or invasion of tumor cells in response to the candidate molecule, to inhibit angiogenesis in the tumor or its metastatic site, to increase the viability of the tumor cells, to increase their extravasation and survival and spread in a metastatic site, and to inhibit expression of a target molecule disclosed herein, for example a molecule that binds and inhibits CCL8 or a CCL8 receptor may cause down-regulation of SIGLEC1 expression and/or inhibit the invasiveness of tumor cells. Tumor cell invasiveness, for example, may be assessed using in vitro assays known in the art, such as a scratch assay or collagen invasion assay. The activity inhibited may be antigen specific T-cell suppression. In addition said inhibitory molecule may block the pro-tumoral phenotype of macrophages and convert it to one that is anti-tumoral. Preferably the CCL8 receptor will be CCR1, CCR2, CCR5 or CCR8. Preferably the therapeutic molecule will be an antagonist of SIGLEC1 or CCL8. Thus the invention provides therapeutic molecules for use in treating cancer that can be found using these methods, for example the therapeutic molecule may be for use in inhibiting metastasis and/or for use in inhibiting recurrence. Preferably the identified molecule that binds SIGLEC1, or CD163, or CCL8 or a CCL8 receptor will be an antibody, further preferably a monoclonal antibody. Preferably the identified molecule that interferes with expression may be a nucleic acid molecule, for example a small interfering RNA for use in RNA interference silencing of SIGLEC1, CD163, CCL8, and/or CCL8 receptor.

### DETAILED DESCRIPTION

The methods of the present invention provide simple tests that may be used in the methods of diagnosing and/or prognosing cancer, predicting efficacy of treatment for cancer, assessing outcome of treatment for cancer, assessing likelihood of metastasis, assessing recurrence of cancer and/or treating cancer. The kits and devices of the invention are useful for conducting the methods of the invention. Methods are also provided for identifying new therapeutic molecules for treating cancer. Cancers to which the invention may be applied include, but are not limited to, breast, lung, brain and colon cancer, particularly breast cancer.

The invention is based on the inventors' significant and surprising finding that the expression of certain biomarkers, namely SIGLEC1, CCL8, CD163, and CD68, in tissue, specifically macrophages, is associated with cancer and in particular cancer that is more likely to spread to other parts of the body (metastasize) and more likely to recur following treatment, and such expression is also associated with a decreased overall life expectancy for the subject.

As discussed elsewhere in the specification, the methods of the invention directed at diagnosis, prognosis, prediction and/or treatment may be carried out using tissue samples, for example tissue samples obtained during diagnostic, preventative, curative, palliative and/or reconstructive surgery. The ability to carry out the methods using this tissue provides advantages, because it provides the clinician with additional clinical indications regarding the (potentially) cancerous tissue. Those clinical indications allow more informed decisions to be made regarding treatment options, and can provide the patient with greater idea as to what they should expect to experience due to their specific cancer.

Of course, in some embodiments the methods of the invention may be used in combination with other methods of diagnosing, prognosing, predicting and/or treating cancer, in which case the combination may advantageously increase specificity and sensitivity compared to use of the other methods on their own, and allow the prioritization of the identification, follow-up and treatment of those most likely to have or develop a more aggressive form of cancer and those most suited to a particular form of treatment.

Thus methods of the invention may allow patients with suspected cancer to be identified swiftly, and guide medical staff to commence appropriate treatment promptly. Furthermore, the methods may allow patients with less aggressive cancer to avoid unnecessarily harsh treatment regimens.

In order to assist the understanding of the present invention, certain terms used herein will now be further defined, and more generally further details of the invention will be given, in the following paragraphs. Generally, terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Biological Sample

The expression level of SIGLEC1, and optionally CD163, CD68, and/or CCL8, is analyzed in a biological sample obtained from a subject. The biological sample is preferably a tissue sample or a derivative thereof, preferably the tissue sample will be tissue removed during diagnostic, for example core needle biopsy or fine needle aspiration, preventative, curative, palliative and/or reconstructive surgery. Preferably the tissue will have been removed from a subject having, or suspected of having, cancer, for example in, or in the vicinity of, the removed tissue.

Preferably the biological sample will comprise, or substantially consist of, stromal tissue that is or was adjacent to the cancerous cells. The inventors have surprisingly found that the methods of the invention may be carried out on the stromal tissue that supports, or has supported, the cancerous cells. Thus, the methods of the invention may involve using stromal tissue that has been removed from a site where cancerous cells are suspected or have been detected in the past, and carrying out methods of the invention on the stromal tissue to obtain a diagnosis, prognosis, or prediction regarding the cancer. An attempt may have been made to remove, e.g. surgically, or destroy, e.g. chemically, the cancerous cells and the methods of the invention may provide an analysis as to how successful that removal (i.e. treatment) has been and what the likely outcome will be for the subject; that treatment may have immediately preceded the removal of the stromal tissue for use in the methods of the invention, or alternatively the stromal tissue may be removed a significant period of time after the treatment, for example weeks, months, or years after the treatment, in which case the methods of the invention may be used, for example, to monitor the likely recurrence or metastasis of the cancer.

Methods of the invention may involve detecting expression levels in tissue samples in which the target molecules have been labelled, for example using immunohistochemistry (IHC), preferably multiplex IHC (mIHC), or fluorescence in situ hybridization (FISH) to detect RNA molecules (RNA FISH). Such methods for labelling target molecules may be included in the methods of the invention, and associated reagents may be included in the kits and devices for use in the methods of the invention, for example reagents required to carry out IHC, mIHC, and/or RNA FISH. Preferably there will be no artificial enrichment of macrophages in the tissue sample prior to the analysis, such that up to 60% of the cells in the tissue sample may be macrophages, for example between 5 and 55% of cells in the tissue. In particularly preferred methods, IHC will be performed to detect and quantify target molecules present in tissue that has been removed before therapeutic treatment and in corresponding tissue that has been removed after therapeutic treatment, with a comparison made of the concentration or number of target molecules present in each tissue sample, in order to detect changes in expression in response to treatment.

Expression levels may be selectively detected in macrophages of the biological sample. Preferably the biological sample, or part thereof, in which the levels are detected will be enriched for macrophages or may substantially consist of macrophages. Thus the sample may be enriched for macrophages or may substantially consist of macrophages, for example at least 75% of the cells in the biological sample may be macrophages, preferably at least 80%, 85%, 90%, 95%, 96%, 97%, 97.5% 98%, 99%, 99.5% or 99.8% of the cells in the sample may be macrophages. It is particularly preferred that at least 97% of the cells in the sample will be macrophages. Suitable methods for enriching samples for macrophages are known to those of skill in the art, for example using FACS sorting or commercially available kits like magnetic cell isolation kits (Miltenyi Biotec), which may make use of selective antibodies to CD163, CD68, CD169 coupled to magnetic beads. Such methods for enrichment may be included in the methods of the invention, and associated reagents may be included in the kits and devices for use in the methods of the invention.

Alternatively or additionally, the step of analyzing the expression levels of the one or more biomarkers may specifically target the macrophages. For example, the analysis may take place on the magnetic beads to which the macrophages specifically attach, such that even though the biological sample may be complete biopsy tissue for example, the expression levels analyzed substantially correspond only to the levels in the macrophages of the sample.

It is preferred that the sample will be enriched for macrophages, or the step of analyzing the levels of the one or more biomarkers will specifically target the macrophages, for example preferably when the expression levels of SIGLEC1 are to be analyzed.

Alternatively or additionally, it may be preferable to take the numbers of macrophages into account when carrying out the analysis of the expression levels of the one or more biomarkers in the sample, so that the analysis will indicate, for example a difference in expression levels of at least one biomarker that may be due to a difference in the relative number of macrophages in the tissue, and/or the analysis may include, for example, an indication of the number or concentration of macrophages present in the sample (for example, whether the tissue from which the sample was taken is relatively enriched for macrophages or not, according to how many macrophages there are in a particular amount of tissue), the expression levels per macrophage, and/or how many of the macrophages present in the sample express the biomarker of interest. Thus in one embodiment the analysis may take place on a section of tissue, and the tissue may be stained, using one or more biomarkers of the invention or otherwise, such that the macrophages present in the tissue are identifiable and the expression levels for SIGLEC1 and/or CCL8, and optionally CD163 and/or CD68, associated with those macrophages can be assessed according to how many macrophages there are expressing each biomarker in a particular area or volume of tissue. In this way, it is possible to identify increases in specific populations of macrophages, which increases in population correlate with a poor outcome for the subject from whom the tissue was taken. For example, an increase in the population of macrophages that are positive for CD163, SIGLEC1 and CCL8 generally indicates a likely poorer outcome.

Particularly where there is no artificial enrichment for macrophages in the sample, the analysis of biomarker levels may reflect the number of macrophages present in the tissue; for example, an increase in the level of one or more biomarkers in the tissue may, at least in part, be due to a larger number of macrophages (TAMs) in the tissue when compared to the number of resident macrophages in an equivalent amount of normal tissue. It is particularly preferred that expression levels of CD163 and/or CD68 in the sample will be analyzed without adjustment of the ratio of macrophages to other cell types in the tissue, such that an increase in expression levels of CD163 and/or CD68 according to a method of the invention may be due, at least in part, to a relative increase in the number of macrophages (TAMs) in the tissue. CD163 and CD68 are pan-macrophage markers (i.e. not specific to TAMs), and quantification of their presence in a tissue may indicate the number of macrophages present in the tissue. Since there is some difference in expression of CD163 and CD68, i.e. there is a population of macrophages that will be positive for CD163 but not CD68, and vice versa, preferably methods of the invention will involve looking at levels of both CD163 and CD68. Therefore, preferably methods of the invention will involve determining the presence of target molecules for analyzing SIGLEC1 and CD163 expression in the sample, or SIGLEC1 and CD68, or CCL8 and CD163, or CCL8 and CD68, or SIGLEC1, CD68 and CD163, or CCL8, CD68 and CD163, or SIGLEC1, CCL8 and CD163, or SIGLEC1, CCL8 and CD68, or SIGLEC1, CCL8, CD68 and CD163. Thus it may not be necessary to enrich for macrophages when preparing a sample for use in the methods of the invention.

The method may involve obtaining a sample of biological material from the subject, or it may be performed on a pre-obtained sample, e.g. one of which has been obtained previously for this or other clinical purposes. Similarly, the biological sample obtained from the subject may be processed before use in methods of the invention, for example to enrich for macrophages or to prepare the tissue in sections on slides for staining, and/or the methods of the invention may include suitable processing steps to enrich for or identify macrophages in the sample, for example through the use of selective magnetic separation systems or suitable tissue sample labelling, such as mentioned above.

In some embodiments the methods of the present invention may make use of multiple biological samples taken from a subject to determine the expression level of the one or more biomarkers.

A Subject

In the context of the methods and medical uses of the present invention, a subject may be anyone requiring the diagnosis, prognosis, prediction and/or treatment for cancer. Suitably the subject may be a mammal, preferably a primate and further preferably a human subject. The subject may be of any sex, for example female or male.

As mentioned elsewhere in the specification, the subject may present with symptoms consistent with cancer and/or they may have already undergone tests that have suggested that they have cancer. Potentially cancerous tissue may be removed from such subjects during diagnostic, curative, palliative and/or reconstructive surgery as described above. For such a subject, the removed tissue may be used in a method of the invention to indicate, for example, the presence of cancer, and optionally the likelihood that the cancer has, or will, metastasize, and/or the likelihood of recurrence of the cancer after it is treated, as explained further below.

Alternatively, the subject may appear to be asymptomatic. Suitably an asymptomatic subject may be a subject who is believed to be at elevated risk of having cancer, for example breast cancer. Such an asymptomatic subject may be one who has a family history of early-onset of cancer, or a genetic risk of cancer such as breast cancer, or who has an increased risk of an age-related cancer. For example, the subject may be a subject considered to be at increased risk of developing breast cancer who has a prophylactic mastectomy, and the breast tissue removed may be used in a method described herein in order to check for the presence of breast cancer.

Levels of Biomarkers

Methods of the invention involve looking at the expression level of the one or more biomarkers of the invention, i.e. one or more biomarkers corresponding to SIGLEC1, and/or one or more biomarkers corresponding to CD163 and optionally one or more biomarkers corresponding to CD68 and/or one or more biomarkers corresponding to CCL8. Preferably the methods involve looking at the expression levels of: SIGLEC1 and CD163; or SIGLEC1 and CD68; or CCL8 and CD68, or CCL8 and CD163, or SIGLEC1, CD163 and CD68; or CCL8, CD163, and CD68, or SIGLEC1, CD163, CD68 and CCL8.

It will be apparent to the skilled person that the above-mentioned biomarkers (SIGLEC1 and/or CCL8) and combinations of biomarkers represent various minimal marker sets, and additional biomarkers can also be included. Alternatively, in some methods, the one or more biomarkers corresponding to SIGLEC1, CD163, CD68 and/or CCL8 may be the only biomarkers for which the expression levels are assessed. However, the methods, kits and devices may also provide for the assessment of control target molecules in the biological sample, where the assessment of the control target molecules allow for the accuracy of the assessment mechanism to be tested.

The invention involves assessing changes in levels for biomarkers, and in preferred embodiments this change is typically differentially upwards for SIGLEC1, CCL8, CD68 and/or CD163 in subjects having a particular clinical indication, preferably a diagnosis that cancer is present, that the cancer is likely to metastasize, that recurrence is likely, and/or that the prognosis or prediction is poor. As discussed above, an increase in overall expression of CD163 and/or CD68 in a tissue sample may generally indicate an increase in the number of macrophages and/or TAMs in the tissue. An increase in the number of macrophages and/or TAMs in the tissue may, of course, anyway indicate that a subject has a particular clinical indication, such as a diagnosis that cancer is present, that the cancer is likely to metastasize, that recurrence is likely, and/or that the prognosis or prediction is poor.

Throughout, biomarkers in the biological sample(s) from the subject are said to be expressed at different levels, or differentially expressed, where they are significantly up- or down-regulated. Depending on the individual biomarker, a cancer diagnosis, prediction or prognosis may be given from a biological sample based on either an increase or decrease in expression level, optionally scaled in relation to sample mean and sample variance, relative to those of subjects not having cancer, or one or more reference values. Clearly, variation in the sensitivity of individual biomarkers, subjects and samples mean that different levels of confidence are attached to each biomarker. Biomarkers of the invention are said to be significantly up- or down-regulated when, optionally after scaling of biomarker expression levels in relation to sample mean and sample variance, they exhibit at least a 1.5-fold change, preferably a 2-fold change, compared with subjects not having cancer or one or more reference values (i.e. a log₂ fold change of greater than 0.58 or less than −0.58, preferably greater than +1 or less than −1). Preferably biomarkers will exhibit a 3-fold change or more compared with the reference value. More preferably biomarkers of the invention will exhibit a 4-fold change or more compared with the reference value. That is to say, in the case of increased expression level (up-regulation relative to reference values), the biomarker level will be more than double that of the reference value. Preferably the biomarker level will be more than 3 times the level of the reference value. More preferably, the biomarker level will be more than 4 times the level of the reference value. Conversely, in the case of decreased expression level (down-regulation relative to reference values), the biomarker level will be less than half that of the reference value. Preferably the biomarker level will be less than one third of the level of the reference value. More preferably, the biomarker level will be less than one quarter of the level of the reference value.

The term “reference value” may refer to a pre-determined reference value, for instance specifying a confidence interval or threshold value for a clinical indication to be allocated to the sample, for example a diagnosis that cancer is present or for prediction of the susceptibility of a subject to treatment, metastasis and/or recurrence. Alternatively, the reference value may be derived from the expression level of a corresponding biomarker or biomarkers in a ‘control’ biological sample, for example a positive (e.g. tissue sample from a patient, the sampled tissue having a cancer diagnosis and/or not being susceptible to treatment and/or leading to metastasis and/or leading to a poor outcome) or negative (e.g. tissue sample from a patient not diagnosed with cancer or a patient diagnosed with a cancer in the sampled tissue that proved susceptible to treatment or a patient diagnosed with cancer in the sampled tissue who had a successful outcome) control. Furthermore, the reference value may be an ‘internal’ standard or range of internal standards, for example a known concentration of a protein, transcript, label or compound within the sample. Alternatively, the reference value may be an internal technical control for the calibration of expression values or to validate the quality of the sample or measurement techniques. This may involve a measurement of one or several transcripts or proteins within the sample which are known to be constitutively expressed or expressed at a known level (e.g. an invariant level). Accordingly, it would be routine for the skilled person to apply these known techniques alone or in combination in order to quantify the level of one or more biomarkers in a sample relative to standards or other transcripts or proteins in order to validate the quality of the biological sample, the assay or statistical analysis.

In preferred methods of the invention the reference values correspond to the levels of the same one or more biomarkers in the same type of tissue, preferably breast tissue, when not associated with cancer i.e. from tissue samples from subjects not having cancer, where the tissue sample comes from the same type of tissue or organ as the suspected cancer; further preferably the reference values for SIGLEC1, CCL8 and/or CD163 will correspond to the levels of the same one or more biomarkers in resident macrophages from the tissue. Thus the reference values may be representative of corresponding values in subjects not having cancer. Preferably therefore, comparison of the expression levels of the one or more biomarkers in the biological sample from the subject with the reference values corresponding to those from a subject not having cancer will show whether there is a difference in expression of the one or more biomarkers relative to the normal tissue and/or resident macrophages in the normal tissue, and an increase in expression of the one or more biomarkers, as explained further below, will be indicative of a diagnosis that cancer is present or of a prediction of decreased susceptibility of a subject to treatment, and/or increased susceptibility to metastasis and/or recurrence.

Alternatively the reference values may correspond to the levels of the one or more biomarkers in samples from subjects who had been diagnosed with cancer and for whom one or more of the clinical details regarding the cancer are known, such as the outcome of treatment of the cancer, whether metastasis occurred, survival rate, and/or the recurrence status. Thus the reference values may be representative of corresponding values in the affected tissue from subjects who have been subsequently successfully treated for cancer, in subjects who have subsequently been unsuccessfully treated for cancer, in subjects who experienced metastasis, in subjects who did not experience metastasis, in subjects who had a substantially reduced lifespan, in subjects who survived for a significant period of time after cancer treatment, and/or in subjects previously successfully treated for whom the cancer has returned. Preferably, in some methods involving providing a prognosis for a subject, providing a treatment for a subject, and/or predicting a subject's response to (a particular) treatment, the reference values may correspond to the levels of the biomarkers in samples from subjects with a particular known prognosis or response to a particular treatment.

Preferably the subjects used to generate the reference values will be “matched” to some extent with those providing the biological sample. For example, if the subject providing the sample is a female suspected of having a cancer then preferably the subjects providing the reference values will also be female. Similarly, if the subject providing the sample is an adolescent male suspected of having cancer then preferably the subjects providing the reference values will also be adolescent males. Thus the subjects providing the samples to which the reference values correspond may be “matched” according to sex and/or age. Alternatively the subjects providing the samples to which the reference values correspond may comprise a range of ages and/or sexes. Also, the tissue from which the reference values are generated may be “matched” to the tissue from which the cancer is known or suspected to originate, or alternatively the reference values may be generated from numerous different tissues. Similarly, preferably the samples used to generate the reference values will be processed in the same way as the interrogated biological samples, according to the methods of the invention, for example with the same methods used to enrich for macrophages and/or interrogate the expression levels of the biomarkers.

The expression level may be an indication of the total number of target molecules present in the analyzed tissue sample, or alternatively the expression level may be an indication of the relative number of target molecules present in the analyzed tissue sample, for example relative to the total number of cells, the number of macrophages, or the size (e.g. volume or area) of the tissue sample that is analyzed; the reference value used will be chosen or calculated to take account of this nature of the expression level. Thus the analysis of expression levels in macrophages of the tissue samples may provide an indication of the number of target molecules, for example mRNA or protein molecules, in the analyzed tissue sample and/or in the macrophages of the tissue sample, and this may be compared to one or more reference values generated by a similar calculation of an indication of the number of target molecules in a tissue sample and/or in the macrophages of a tissue sample from similarly processed tissue of a known clinical indication. Alternatively, in particularly preferred embodiments of the methods of the invention the analysis of expression levels involves determining how many cells within a certain area of the tissue sample have the one or more target molecules (for example proteins) present, for example how many cells per mm² or per cm² of a histological section of the tissue sample have the one or more target molecules present, and this may be compared to one or more reference values generated by a similar calculation of how many cells within a certain area of similarly processed tissue of a known clinical indication have the one or more target molecules present, or alternatively it may be compared to a pre-determined reference value.

It will be apparent to the skilled person that there is considerable freedom to interpret and process data obtained by the methods of the present invention, and how to interpret or act upon the results. It may be desirable, for example, to prioritise sensitivity or specificity, or to optimise positive predictive value or negative predictive value. Depending on the context in which the method is performed, the skilled person can therefore select appropriate methods of interpreting the results. For example, the results for different markers can be weighted in different ways, different threshold abundance levels can be applied, various statistical analyses can be applied, and one or more indicators of a potentially dubious test result can be determined.

The person skilled in the art is free to formulate a wide range of calculations, e.g. via suitable algorithms, in order to obtain a desired result from processing data obtained from the methods of the present invention. There involves routine application of well-known statistical analysis techniques. Similarly, routine assay techniques can be used to detect the target molecules, such as IHC, mIHC, and/or RNA FISH with tissue samples, and counting of the detected molecules may be carried out in an automated way, for example using machine learning or artificial intelligence based methods; the skilled person will be aware of such techniques from the art.

SIGLEC1 (sialic acid binding Ig like lectin 1; Ensembl ID ENSG00000088827; also called CD169) is located on chromosome 20 and it encodes a member of the immunoglobulin superfamily. The terms SIGLEC1 and CD169 are used interchangeably herein. The inventors have surprisingly found that this gene is significantly overexpressed in TAMs found in cancer, compared to the expression in resident macrophages not associated with cancer, and that its expression is associated with a poorer prognosis in terms of metastasis and recurrence. Therefore in methods of the invention in which the expression levels of SIGLEC1 are analyzed, it is preferred that significant up-regulation of the expression level of SIGLEC1 in a sample from a subject is associated with the subject having a particular clinical indication, preferably a diagnosis that cancer is present, an indication that metastasis is likely, an indication that recurrence is likely and/or an indication of a poor prognosis or prediction. For example, a log₂ fold change of at least 0.58, for example a log₂ fold change of at least 1, 1.5, 2.0, 2.5, 3.0, 3.5, 4.0, 4.5, 5.0, 5.1, 5.2, 5.3, 5.4, 5.5, 5.6, 5.7, 5.8, 5.9, or 6.0 in a sample compared to one or more reference values may be indicative of the subject having a particular clinical indication, preferably a diagnosis that cancer is present. The skilled person will appreciate that the relative expression levels of SIGLEC1 will depend on the reference values used in the comparison; however, in preferred methods the reference values will correspond to the levels of the biomarkers in resident macrophages from the same type of tissue from subjects not having cancer, and a log₂ fold change of at least 2, preferably at least 2.5, 3.0, 3.5, 4.0, 4.5, 5.0 or 5.5, in expression levels of SIGLEC1 will be indicative of the subject having a particular clinical indication, preferably a diagnosis that cancer is present, an indication that metastasis is likely, an indication that recurrence is likely and/or an indication of a poor prognosis or prediction.

In the experiments described below, the inventors show that SIGLEC1 expression is increased in TAMs, particularly TAMs associated with a poor prognosis. This is shown at both the RNA and the protein level. Therefore the skilled person will appreciate that SIGLEC1 expression can similarly be analyzed in the methods of the invention by looking at nucleic acid and/or protein levels. The skilled person will also appreciate, and it is illustrated below, that when nucleic acid levels of SIGLEC1 are analyzed it is preferable to analyze the expression in samples enriched for macrophages, and/or to specifically analyze the expression in the macrophages of the sample as explained above, whilst when protein levels of SIGLEC1 are analyzed this is less important so that it is preferable to analyze samples, for example histological sections, of tissue that retains its cell ratio and structure.

Suitably, the methods may involve analyzing the number of cells (macrophages) in the tissue sample that express SIGLEC1 protein, i.e. that are CD169 positive (CD169+). Preferably in such methods, the reference value will be 5, 10, 15, 20, 25, 30, 35, 40, 45, 50, 60, 70, or 75 CD169+ cells per mm² of tissue section, such that an expression level of greater than 5, 10, 15, 20, 25, 30, 35, 40, 45, 50, 60, 70, or 75 CD169+ cells per mm² of tissue section will be indicative of the subject having a particular clinical indication, preferably a diagnosis that cancer is present, an indication that metastasis is likely, an indication that recurrence is likely and/or an indication of a poor prognosis or prediction. Further preferably in such methods, the reference value will be 25 CD169+ cells per mm² of tissue section, such that an expression level of greater than 25 CD169+ cells per mm² of tissue section will be indicative of the subject having a particular clinical indication, preferably a diagnosis that cancer is present, an indication that metastasis is likely, an indication that recurrence is likely and/or an indication of a poor prognosis or prediction. Generally the greater the increase in the number of CD169+ cells per mm² of tissue section, compared to reference values based on similar analysis of normal tissue or cancer tissue having a good prognosis, the greater the likelihood that the subject has a particular clinical indication, preferably a diagnosis that cancer is present, an indication that metastasis is likely, an indication that recurrence is likely and/or an indication of a poor prognosis or prediction, i.e. higher numbers of CD169+ cells per mm² of tissue section are associated with a poorer prognosis. When analysis of the number of cells per mm² of tissue section is carried out in this way, it is preferred that the expression level is calculated based on the mean value of the number of positive cells in a particular size of tissue section, such as 5, 10, 15, or 20 mm² of tissue section, since the skilled person will appreciate that generally the accuracy of the analysis as being representative of the tissue section will increase as the area of tissue used for the analysis also increases.

CCL8 (C-C motif chemokine ligand 8; Ensembl ID ENSG00000108700) is located on chromosome 17 and it encodes a cytokine that displays chemotactic activity for monocytes, lymphocytes, basophils and eosinophils. The inventors have surprisingly found that this gene is significantly overexpressed by TAMs found in cancer, compared to the expression by resident macrophages not associated with cancer. Therefore in methods of the invention in which the expression levels of CCL8 are analyzed, it is preferred that significant up-regulation of the expression level of CCL8 in a sample from a subject is associated with the subject having a particular clinical indication, preferably a diagnosis that cancer is present, an indication that metastasis is likely, an indication that recurrence is likely and/or an indication of a poor prognosis or prediction. For example, a log₂ fold change of at least 0.58, for example a log₂ fold change of at least 1, 1.5, 2.0, 2.5, 3.0, 3.5, 4.0, or 4.5 in a sample compared to one or more reference values may be indicative of the subject having a particular clinical indication, preferably a diagnosis that cancer is present. The skilled person will appreciate that the relative expression levels of CCL8 will depend on the reference values used in the comparison; however, in preferred methods the reference values will correspond to the levels of the biomarkers in samples from subjects not having cancer, and a log₂ fold change of at least 1.5, preferably at least 2.5, 3.0, or 3.5 in expression levels of CCL8 will be indicative of the subject having a particular clinical indication, preferably a diagnosis that cancer is present, an indication that metastasis is likely, an indication that recurrence is likely and/or an indication of a poor prognosis or prediction.

In the experiments described below, the inventors show that CCL8 expression at the RNA level is increased in TAMs, particularly TAMs associated with a poor prognosis, and that CCL8 is produced in these TAMs and then secreted into the surrounding tissues. This is shown at both the RNA and the protein level. Therefore the skilled person will appreciate that CCL8 expression can be analyzed in the methods of the invention by looking at either nucleic acid or protein levels. The skilled person will also appreciate, and it is illustrated below, that when nucleic acid levels of CCL8 are analyzed, for example using RNA FISH or intracellular FACS, it is preferable to analyze the expression in samples enriched for macrophages, and/or to specifically analyze the expression in the macrophages of the sample as explained above, whilst when protein levels of CCL8 are analyzed this is less important so that it is preferable to analyze samples, for example histological sections, of whole tissue. Secreted CCL8 protein can be detected in media using an enzyme-linked immunosorbent assay (ELISA).

CD163 (CD163 molecule; Ensembl ID ENSG00000177575) is located on chromosome 12 and it encodes a member of the scavenger receptor cysteine-rich (SRCR) superfamily. This gene is significantly overexpressed in cancer tissue compared to the expression in the same tissue not associated with cancer, and its expression is associated with a poorer prognosis in terms of metastasis and recurrence. Without intending to be bound by any theory, this is most likely due to an increase in the number of macrophages (i.e. TAMs) in the cancer tissue, since CD163 is a pan-macrophage marker. Therefore in methods of the invention in which the expression levels of CD163 are analyzed, it is preferred that significant up-regulation of the expression level of CD163 in a sample from a subject is associated with the subject having a particular clinical indication, preferably a diagnosis that cancer is present, an indication that metastasis is likely, an indication that recurrence is likely and/or an indication of a poor prognosis or prediction. It is also preferred that any analysis is carried out on tissue samples, or derivatives thereof, that have not been enriched for macrophages. For example, a log₂ fold change of at least 0.58, for example a log₂ fold change of at least 1, 1.5, 2.0, or 3.0 in a sample compared to one or more reference values may be indicative of the subject having a particular clinical indication, preferably a diagnosis that cancer is present. The skilled person will appreciate that the relative expression levels of CD163 will depend on the reference values used in the comparison; however, in preferred methods the reference values will correspond to the levels of the biomarker in samples from subjects not having cancer, and a log₂ fold change of at least 0.58, preferably at least 1.0 in expression levels of CD163 will be indicative of the subject having a particular clinical indication, preferably a diagnosis that cancer is present, an indication that metastasis is likely, an indication that recurrence is likely and/or an indication of a poor prognosis or prediction.

In the experiments described below, the inventors show that CD163 expression at the RNA level is increased in cancer tissue, particularly cancer tissue with a poor prognosis. This is shown at both the RNA and the protein level. This is probably due to the expression of CD163 in macrophages, and the infiltration of macrophages into cancer tissues, resulting in an associated increased expression of CD163 in the cancer tissue. Therefore the skilled person will appreciate that CD163 expression can be analyzed in the methods of the invention by looking at either nucleic acid or protein levels, but it is particularly preferred that any such analysis will be in the context of a representative section of the tissue sample rather than a sample enriched for macrophages or a sample in which only the macrophages are analyzed. By “representative section” it is meant some or all of the tissue sample in which the ratio of macrophage to tissue of other cell types is substantially maintained.

Suitably, the methods may involve analyzing the number of cells (macrophages) in the tissue sample that express CD163 protein, i.e. that are CD163 positive (CD163+). Preferably in such methods, the reference value will be 0, 2, 4, 6, 7 or 80 CD163+ cells per mm² of tissue section, such that an expression level of greater than 10, 20, 30, 40, 45, 50, 55, 60, 65, 70, 75, or 80 and up to 300 CD163+ cells per mm² of tissue section will be indicative of the subject having a particular clinical indication, preferably a diagnosis that cancer is present, an indication that metastasis is likely, an indication that recurrence is likely and/or an indication of a poor prognosis or prediction. For example, an expression level of up to, at most, 10 cells per mm² may be considered as normal or pre-cancerous tissue; expression of above 10 cells per mm² up to 250 cells per mm² may be considered a cancerous tissue. Further preferably in such methods, the reference value will be 5 CD163+ cells per mm² of tissue section, such that an expression level of greater than 5 CD163+ cells per mm² of tissue section will be indicative of the subject having a particular clinical indication, preferably a diagnosis that cancer is present, an indication that metastasis is likely, an indication that recurrence is likely and/or an indication of a poor prognosis or prediction. Generally the greater the increase in the number of CD163+ cells per mm² of tissue section, compared to reference values based on similar analysis of normal tissue or cancer tissue having a good prognosis, the greater the likelihood that the subject has a particular clinical indication, preferably a diagnosis that cancer is present, an indication that metastasis is likely, an indication that recurrence is likely and/or an indication of a poor prognosis or prediction, i.e. higher numbers of CD163+ cells per mm² of tissue section are associated with a poorer prognosis. When analysis of the number of cells per mm² of tissue section is carried out in this way, it is preferred that the expression level is calculated based on the mean value of the number of positive cells in 1, 2, 3, 4, 5, 10, 15, 20, 25 or 30 mm² of tissue section, since the skilled person will appreciate that generally the accuracy of the analysis as being representative of the whole tissue section will increase as the area of tissue used for the analysis also increases. It is particularly preferred that the expression level is calculated across substantially the whole tissue section, or at least most of it, for example one or more cm² of tissue, and this may be carried out using generated machine learning or artificial intelligence techniques to capture the amount of labelling (staining).

CD68 (CD68 molecule; Ensembl ID ENSG00000129226) is located on chromosome 17 and it encodes a transmembrane glycoprotein that is a member of the lysosomal/endosomal-associated membrane glycoprotein (LAMP) family and the scavenger receptor family. This gene is significantly overexpressed in cancer tissue compared to the expression in the same tissue not associated with cancer, and its expression is associated with a poorer prognosis in terms of metastasis and recurrence. Without intending to be bound by any theory, this is most likely due to an increase in the number of macrophages (i.e. TAMs) in the cancer tissue, since CD68 is a pan-macrophage marker. Therefore in methods of the invention in which the expression levels of CD68 are analyzed, it is preferred that significant up-regulation of the expression level of CD68 in a sample from a subject is associated with the subject having a particular clinical indication, preferably a diagnosis that cancer is present, an indication that metastasis is likely, an indication that recurrence is likely and/or an indication of a poor prognosis or prediction. It is also preferred that any analysis is carried out on tissue samples, or derivatives thereof, that have not been artificially enriched for macrophages. For example, a log₂ fold change of at least 0.58, for example a log₂ fold change of at least 1, 1.5, 2.0, or 3.0 in a sample compared to one or more reference values may be indicative of the subject having a particular clinical indication, preferably a diagnosis that cancer is present. The skilled person will appreciate that the relative expression levels of CD68 will depend on the reference values used in the comparison; however, in preferred methods the reference values will correspond to the levels of the biomarker in samples from subjects not having cancer, and a log₂ fold change of at least 0.58, preferably at least 1.0 in expression levels of CD68 will be indicative of the subject having a particular clinical indication, preferably a diagnosis that cancer is present, an indication that metastasis is likely, an indication that recurrence is likely and/or an indication of a poor prognosis or prediction.

Without wishing to be bound by any theory, increased CD68 expression in cancer tissue, particularly cancer tissue with a poor prognosis, is probably due to the expression of CD68 in macrophages, and the infiltration of macrophages into cancer tissues, resulting in an associated increased expression of CD68 in the cancer tissue. Therefore the skilled person will appreciate that CD68 expression can be analyzed in the methods of the invention by looking at either nucleic acid or protein levels, but it is particularly preferred that any such analysis will be in the context of a representative section of the tissue sample rather than a sample artificially (i.e. as part of the processing of the tissue sample as opposed to naturally whilst the tissue is in situ) enriched for macrophages or a sample in which only the macrophages are analyzed. By “representative section” it is meant some or all of the tissue sample in which the ratio of macrophage to tissue of other cell types is substantially maintained.

Suitably, the methods may involve analyzing the number of cells (macrophages) in the tissue sample that express CD68 protein, i.e. that are CD68 positive (CD68+). Preferably in such methods, the reference value will be 0, 2, 4, 6, 7 or 80 CD68+ cells per mm² of tissue section, such that an expression level of greater than 10, 20, 30, 40, 45, 50, 55, 60, 65, 70, 75, or 80 and up to 300 CD68+ cells per mm² of tissue section will be indicative of the subject having a particular clinical indication, preferably a diagnosis that cancer is present, an indication that metastasis is likely, an indication that recurrence is likely and/or an indication of a poor prognosis or prediction. Further preferably in such methods, the reference value will be 5 CD68+ cells per mm² of tissue section, such that an expression level of greater than 5 CD68+ cells per mm² of tissue section will be indicative of the subject having a particular clinical indication, preferably a diagnosis that cancer is present, an indication that metastasis is likely, an indication that recurrence is likely and/or an indication of a poor prognosis or prediction. Generally the greater the increase in the number of CD68+ cells per mm² of tissue section, compared to reference values based on similar analysis of normal tissue or cancer tissue having a good prognosis, the greater the likelihood that the subject has a particular clinical indication, preferably a diagnosis that cancer is present, an indication that metastasis is likely, an indication that recurrence is likely and/or an indication of a poor prognosis or prediction, i.e. higher numbers of CD68+ cells per mm² of tissue section are associated with a poorer prognosis. When analysis of the number of cells per mm² of tissue section is carried out in this way, it is preferred that the expression level is calculated based on the mean value of the number of positive cells in 1, 2, 3, 4, 5, 10, 15, 20, 25 or 30 mm² of tissue section, since the skilled person will appreciate that generally the accuracy of the analysis as being representative of the whole tissue section will increase as the area of tissue used for the analysis also increases. It is particularly preferred that the expression level is calculated across substantially the whole tissue section, or at least most of it, for example one or more cm² of tissue, and this may be carried out using generated machine learning or artificial intelligence techniques to capture the amount of labelling (staining).

The biological samples are analyzed to determine the expression levels of one or more of the biomarkers. “Gene expression”, or more simply “expression” is the process by which information from a gene is used in the synthesis of a functional gene product, such as a protein or non-coding RNA (ncRNA). As used herein, the term “expression” includes RNA (for example mRNA) transcription. Thus suitably the expression level for a biomarker may be determined by looking at the amount of a target molecule selected from the group consisting of the protein expressed from the biomarker and a polynucleotide molecule encoding the biomarker, or a nucleic acid complementary thereto. The target molecule may be a nucleic acid molecule, and preferably an RNA molecule, for example mRNA, transcribed from the biomarker or a cDNA molecule corresponding and complementary thereto, such that the expression levels of mRNA for the biomarker(s) can be analyzed. Preferably, the target molecule(s) may be a protein molecule(s), and further preferably the protein molecule(s) may be detected, for example on tissue sections, using IHC, mIHC or immunofluorescence techniques generally.

Determining the abundance of more than one biomarker can be preferable to a single biomarker as it can allow for a more reliable or powerful test. This can occur for many reasons, e.g. because combining information about a plurality of markers reduces the risk that a single biomarker might have an altered abundance because of an unrelated cause and unduly skew the result, and because changes in a broader pattern of abundance levels can be highly informative. Also, as explained herein, the biomarkers of the invention act as biomarkers in a synergistic fashion, in that the power of the biomarkers in combination is greater than would be expected from an additive effect of the power of each individual biomarker.

Therefore in some embodiments of methods of the invention, the expression levels of SIGLEC1 and CCL8 are analyzed. Preferably the analysis is of SIGLEC1 and CCL8 mRNA levels in the tissue sample, preferably wherein the tissue sample is enriched for macrophages or the analysis is targeted at the macrophages in the tissue sample, and comparison is made to mRNA levels in corresponding tissue sample that is not cancerous, and a log₂ fold change as disclosed above, for example of at least 0.58, is indicative of the subject having a particular clinical indication, preferably a diagnosis that cancer is present, an indication that metastasis is likely, an indication that recurrence is likely and/or an indication of a poor prognosis or prediction.

In some embodiments of methods of the invention, the expression levels of SIGLEC1 and CD163 are analyzed. The inventors have surprisingly found that the analysis of SIGLEC1 and CD163 expression levels as biomarkers for a poor prognosis, in particular a poor prognosis with respect to metastasis, recurrence, and overall survival, is synergistic, in that the use of both SIGLEC1 and CD163 as biomarkers together is more informative that the additive effect of using the two biomarkers separately and independently. Therefore preferably the analysis according to the methods of the invention is of SIGLEC1 and CD163 protein levels in the tissue sample, further preferably wherein the expression level is provided as the numbers of SIGLEC1 and/or CD163 positive cells per area, such as cm² mm², of tissue sample section, and comparison is made to reference values which are corresponding SIGLEC1 and CD163 protein levels in tissue from patients having a known clinical indication, for example tissue not associated with cancer, cancer tissue that was subsequently successfully treated and/or the cancer did not recur and/or from which there was no metastasis, or comparison is made to pre-determined reference values. In such preferred methods a significant increase in the number of cells positive for both SIGLEC1 and CD163 (CD163+/CD169+) may be indicative of cancer, and optionally likely recurrence, likely distal metastasis and/or a poorer prognosis or prediction. Preferably in such methods the reference value will be 5, 10, 15, 20, 25, 30, 35, or 40 CD163+/CD169+ cells per mm² of tissue section, such that an expression level of greater than 5, 10, 15, 20, 25, 30, 35, or 40 CD163+/CD169+ cells per mm² of tissue section will be indicative of the subject having a particular clinical indication, preferably a diagnosis that cancer is present, an indication that metastasis is likely, an indication that recurrence is likely and/or an indication of a poor prognosis or prediction. Further preferably in such methods, the reference value will be 25 CD163+/CD169+ cells per mm² of tissue section, such that an expression level of greater than 25 CD163+/CD169+ cells per mm² of tissue section will be indicative of the subject having a particular clinical indication, preferably a diagnosis that cancer is present, an indication that metastasis is likely, an indication that recurrence is likely and/or an indication of a poor prognosis or prediction. Generally the greater the increase in the number of CD163+/CD169+ cells per mm² of tissue section, compared to reference values based on similar analysis of normal tissue or cancer tissue having a good prognosis, the greater the likelihood that the subject has a particular clinical indication, preferably a diagnosis that cancer is present, an indication that metastasis is likely, an indication that recurrence is likely and/or an indication of a poor prognosis or prediction, i.e. higher numbers of CD163+/CD169+ cells per mm² of tissue section are associated with a poorer prognosis. When analysis of the number of cells per mm² of tissue section is carried out in this way, it is preferred that the expression level is calculated based on the mean value of the number of positive cells in 1, 2, 3, 4, 5, 10, 15, or 20 mm² of tissue section, since the skilled person will appreciate that generally the accuracy of the analysis as being representative of the whole tissue section will increase as the area of tissue used for the analysis also increases. It is particularly preferred that the expression level is calculated across substantially the whole tissue section, or at least most of it, for example one or more cm² of tissue, and this may be carried out efficiently using generated machine learning or artificial intelligence techniques to capture the amount of labelling (staining).

In particularly preferred methods, the expression levels of SIGLEC1, CCL8 and CD163 are analyzed. This is because the inventors have surprisingly found that CCL8 expression together with CD163 expression and SIGLEC1 expression shows greater correlation with poor survival than the two factors alone. The expression levels for each of the biomarkers may be carried out simultaneously, sequentially or separately. The target molecules considered in the analysis may be of the same type, e.g. nucleic acid or protein, for each of the biomarkers, or alternatively different types of target molecules may be considered, e.g. protein for CD163 and SIGLEC1 and nucleic acid (such as mRNA or corresponding cDNA) for CCL8. Preferably the analysis for SIGLEC1 and CCL8 may focus on expression in the macrophages of the tissue sample, e.g. by enriching the tissue sample for macrophages before carrying out the analysis, whilst the analysis for CD163 may involve analyzing the expression in a representative sample of cells from the tissue sample, i.e. without any artificial enrichment or specific focus on the macrophages in the sample. In particularly preferred methods, FISH is used to analyze expression levels of the biomarkers.

The levels of the one or more target molecules, which are representative of expression of the one or more biomarkers in the biological sample, may be investigated for example using specific binding partners, polymerase chain reaction (PCR) and/or sequencing techniques, particularly high-throughput sequencing techniques. The binding partners may be selected from the group consisting of complementary nucleic acids, aptamers, and antibodies or antibody fragments. Preferably the levels of the one or more biomarkers in the biological sample are investigated using either antibodies (or antibody fragments) specific for a protein target molecule or a nucleic acid probe having a sequence which is complementary to the sequence of the relevant mRNA or cDNA against which it is targeted. Techniques of particular interest for measuring expression levels of biomarkers include IHC, mIHC, the nCounter® platform from nanoString®, CODEX (CO-Detection by indexing) multiplexed imaging and spatial transcriptomics.

Suitable classes of binding partners for any given biomarker will be apparent to the skilled person, and are discussed further below. The expression levels of the biomarkers in the biological sample may be detected by direct assessment of binding between the target molecules and binding partners. The levels of the biomarkers in the biological sample may be detected using a reporter moiety attached to a binding partner. Preferably the reporter moiety is selected from the group consisting of fluorophores; chromogenic substrates; and chromogenic enzymes.

Methods of Diagnosis, Prognosis, Prediction and Treatment

As explained in the Experimental Results section, the methods of the invention are able to distinguish at least between samples from individuals with and without cancer, and stratify patients according to recurrence, distal metastasis and overall survival. Therefore the term “clinical indication” should be interpreted broadly to refer to clinical details that may generally be associated with a particular tissue sample known as comprising or potentially comprising cancer. In particular “a clinical indication” may refer to the presence or absence of cancer generally, a good response to treatment, a poor response to treatment, a poor survival rate, a good survival rate, a good prognosis/outcome, an intermediate prognosis/outcome, a poor prognosis/outcome, the presence of local and/or distant metastasis, the absence of local and/or distant metastasis, and/or recurrence of the cancer following treatment or no recurrence of the cancer following treatment.

Therefore in some methods of the invention, the clinical indication is of whether cancer is present or not in the tissue of the subject. This diagnosis may be made, for example, by using reference values corresponding to, or having a defined relationship with, the biomarker expression in tissue samples from patients not having cancer; the differential expression for a clinical indication of cancer being present when such reference values are used is indicated above. Alternatively, the diagnosis may be made, for example, by using reference values corresponding to, or having a defined relationship with, biomarker expression in tissue samples from patients having cancer, such that a clinical indication of cancer being present could be made based on the lack of significant differential expression, or the absence of a particular decrease in expression, when such reference values are used.

Alternatively or additionally, the clinical indication comprises an indication of whether the cancer is likely to be associated with local and/or distant metastasis. This indication may be made, for example, by using reference values corresponding to, or having a defined relationship with, the biomarker expression in tissue samples from patients not having cancer; the differential expression for a clinical indication of metastasis being likely when such reference values are used is indicated above. Alternatively, the indication may be made, for example, by using reference values corresponding to, or having a defined relationship with, biomarker expression in tissue samples from patients having a cancer that had metastasized, such that a clinical indication of a likelihood of metastasis could be made based on the lack of significant differential expression, or the absence of a particular decrease in expression, when such reference values are used.

Alternatively or additionally, the clinical indication comprises an indication of whether the cancer is likely to recur following treatment. This indication may be made, for example, by using reference values corresponding to, or having a defined relationship with, the biomarker expression in tissue samples from patients not having cancer; the differential expression for a clinical indication of recurrence being likely when such reference values are used is indicated above, preferably wherein the expression is four-fold or above that of the corresponding tissue from patients not having cancer. Alternatively, the indication may be made, for example, by using reference values corresponding to, or having a defined relationship with, biomarker expression in tissue samples from patients having a cancer that did subsequently recur, such that a clinical indication of a likelihood of recurrence could be made based on the lack of significant differential expression, or the absence of a particular decrease in expression, when such reference values are used.

Alternatively or additionally, the clinical indication comprises an indication of the likelihood (probability) that the subject will survive (such as for one, two, three, four or five years). This indication may be made, for example, by using reference values corresponding to, or having a defined relationship with, the biomarker expression in tissue samples from patients not having cancer; the differential expression for a clinical indication of a likely shortened survival period when such reference values are used is indicated above. Alternatively, the indication may be made, for example, by using reference values corresponding to, or having a defined relationship with, biomarker expression in tissue samples from patients that only survived for a short period, such that a clinical indication of likely survival period could be made based on the lack of significant differential expression, or the magnitude of any differential expression.

The term “diagnosing” or “diagnosis” as used herein in the context of cancer should be taken as allowing a distinction to be made regarding a tissue sample; the term is used to mean an indication of the presence or absence of cancer and/or an indication of particulars of the disease.

The term “prognosis” as used herein refers to the likelihood of the clinical outcome for a subject having cancer, and is a representation of the likelihood (probability) that the subject will survive (such as for one, two, three, four or five years) and/or the likelihood (probability) that the tumor will progress in grade and/or metastasize. The term “prediction” is used herein to refer to the likelihood that a patient will respond either favorably or unfavorably to a therapy, drug or set of drugs, and also the extent of those responses. The prognostic and predictive methods of the invention can be used clinically to make treatment decisions by choosing the most appropriate treatment modalities for any particular patient. The methods are thus valuable tools in predicting if a patient is likely to respond favorably to a treatment regimen, such as surgical intervention, chemotherapy with a given drug or drug combination, and/or radiation therapy, as explained further below. Methods of prognosis or prediction may involve, for example, comparison of biomarker expression in the tissue sample from the subject with biomarker expression in tissue samples from patients having a particular known outcome; difference or similarity in expression can then be used to decide whether the subject is likely to have the same outcome from a prognosis and/or prediction perspective.

In preferred methods, the greater the extent of the increased expression of one, two or all three, of the biomarkers of the invention compared to reference values from subjects not having cancer, the poorer the prognosis for the subject and/or the less likely they are to respond to the cancer treatment.

Other physical or biological measurements may be taken, or tests carried out, in conjunction with the measurement of biomarker expression levels as part of the methods of the invention. Preferably the methods of the invention, or at least preferably those that do not involve treatment of the subject, are performed in vitro and/or ex vivo and/or are not practiced on the subject's body. For the avoidance of doubt, it should be noted that the present invention can be used for both initial diagnosis of cancer and for ongoing monitoring of cancer, e.g. indicating the continued presence of cancer despite treatment (response to, or outcome following, treatment) or indicating the presence of cancer after a period of being “cancer free” following treatment (assessing recurrence).

Where methods are described herein as indicating a “poor” diagnosis, prognosis, or outcome, this is used to mean that the cancer is clinically associated with more developed, advanced, aggressive and/or extensive disease and so a poor clinical outcome. A “poor” prediction means that the cancer is clinically associated with an incomplete response to a particular treatment, so that the cancer is not completely removed and/or it is likely to recur, leading to a poor clinical outcome. Clinical outcome refers to the health status of a patient following treatment for a disease or disorder, or in the absence of treatment, and so clinical outcomes include, but are not limited to, an increase in the length of time until death, a decrease in the length of time until death, an increase in the chance of survival, an increase in the risk of death, survival, disease-free survival, chronic disease, metastasis, advanced or aggressive disease, disease recurrence, death, and favorable or poor response to therapy. For example, a method indicating a poor clinical diagnosis, prognosis, or outcome may indicate a higher grade of cancer, a lower probability of response to treatment, a greater probability of recurrence following treatment, and/or a greater probability of a reduced life expectancy. In comparison, a “good” diagnosis, prognosis, or outcome, is used to mean that the cancer is clinically associated with less developed, advanced, aggressive and/or extensive disease and so a good clinical outcome. For example, it may indicate a lower grade of cancer, a higher chance of response to treatment, a lower chance of recurrence following treatment, and/or minimal impact of the cancer on life expectancy.

The methods of the invention may be used to provide a clinical indication, for example diagnose cancer, in a subject showing symptoms consistent with such disease. Alternatively, the methods of the invention may be used to diagnose cancer in a subject that appears asymptomatic. Cancer may be asymptomatic, for example, during the early stages of recurrence of the disease.

As used herein the term “cancer” includes: cancer generically; groups or sub-groups of cancers originating from specific organs, tissues and/or cell types; cancer originating from a specific organ, tissue and/or cell type; and cancers of unknown primary origin. For example, a method of the invention may indicate that the subject has cancer, without the site or origin of the cancer being known or indicated, or alternatively a method of the invention may indicate that the subject has a more specific type of cancer, such as breast cancer. Indeed it is a remarkable feature of the present invention that these biomarkers have broad utility for detecting and providing information about many different types of cancers. The specificity of the cancer diagnosis given may depend, for example, on whether the subject has any symptoms and what those symptoms are, which may indicate a suspected originating site for the cancer, and/or further measurements taken or tests carried out in order to indicate a likely origin of the cancer; said measurements or tests may form part of the methods of the invention, or alternatively may be carried out additionally, simultaneously with or separately from the methods of the invention, before or after the methods of the invention. Such measurements or tests that may be part of the methods of the invention, or additional to it, include further blood tests, X-rays, CT scans and endoscopy.

The cancer detected and/or indicated in the present invention may be breast cancer, endometrial cancer, ovarian cancer, prostate cancer, pancreatic cancer, thyroid cancer, cervical cancer, bladder cancer, blastoma, brain cancer and gliomas, bowel cancer, gastric cancer, head and neck cancer, kidney cancer, liver cancer, lung cancer, mesothelioma, melanoma, oral cancer, pituitary cancer, skin cancer, soft tissue cancer, testicular cancer, uterine cancer, heart cancer, and/or eye cancer. Preferably the methods, kits and devices of the invention will be for subjects having, or suspected of having, a solid tumor cancer, for example a carcinoma. Preferably the solid tumor cancer will not be a sarcoma. Preferably the cancer will be breast, lung, or colon cancer, further preferably breast cancer.

Preferably the methods, kits and devices of the invention will not be for subjects having, or suspected of having, a blood cancer, for example preferably the cancer will not be a leukemia, and/or a myeloma. It is particularly preferred that the cancer will not be a myeloid leukemia, for example monocytic leukemia, or a lymphocytic leukemia.

It is preferred in the present invention that the cancer is lung cancer, colon cancer, or a hormone-related cancer, for example breast cancer, endometrial cancer, ovarian cancer, prostate cancer, pancreatic cancer, and thyroid cancer. In some embodiments, the cancer is not hepatocellular carcinoma. It is particularly preferred that it is lung cancer, colon cancer, or an estrogen-dependent cancer, for example a cancer selected from breast cancer, endometrial cancer and ovarian cancer. It is most preferred that the cancer is lung, colon or breast cancer. Breast cancer includes, for example, ductal carcinoma in situ (DCIS), invasive ductal carcinoma, invasive lobular carcinoma, or inflammatory breast cancer. Lung cancer includes small cell lung cancer, and non-small cell lung cancer, such as adenocarcinoma, squamous cell cancer and large cell carcinoma, Pancoast tumors and mesothelioma. Colon cancer includes adenocarcinomas, squamous cell tumors, and carcinoid tumors. In particularly preferred embodiments, the methods can be used to identify the sub-type to which the cells of the tissue sample belong, for example ER+, Her2+, basal, luminal, etc., breast cancer.

The cancer may be primary or metastatic, and may be a recurrent cancer. The methods may suitably comprise comparing the results obtained with the results obtained in an equivalent (typically identical) procedure carried out previously on a biological sample of tissue from the same subject. In this way, for example, the development or treatment of cancer in a subject may be monitored.

The methods of treatment may involve any of the treatments known in the art for the cancer diagnosed, for example one or more treatments selected from the group consisting of surgery, radiation therapy, chemotherapy, immunotherapy, hormone therapy, and targeted therapy. The therapy may, for example, be used to remove the entire tumor, to debulk the tumor, and/or to ease the cancer symptoms.

Surgery involves removing or destroying tumor tissue and may be open or minimally invasive. It may include, for example, the use of sharp tools to cut the body, cryosurgery, lasers, hyperthermia and/or photodynamic therapy.

Radiation therapy involves the use of high doses of radiation to kill cancer cells and shrink tumors. Treatment using radiation therapy in accordance with the invention includes the use of external beam radiation therapy, where an external source is used to aim radiation at the affected part(s) of the body, internal radiation therapy (brachytherapy), where a solid or liquid radiation source is put into the body, and systemic radiation therapy. Radiation therapies of use in embodiments of the invention include the use of external x-rays or gamma rays, interstitial brachytherapy, intracavitary brachytherapy, episcleral brachytherapy, radioactive iodine, samarium-153-lexidronam (Quadramet) and strontium-89 chloride (Metastron).

Chemotherapy involves the use of chemicals that target the fast dividing cancer cells. It may be used on its own or in combination with other cancer therapies. Chemotherapy drugs of use in embodiments of the invention include one or more of Abraxane (Abraxane), Amsacrine (Amsidine), Azacitidine (Vidaza), Bendamustine, (Levact), Bleomycin, Busulfan (Busilvex, Myleran), Cabazitaxel (Jevtana), Capecitabine (Xeloda), Carboplatin, Carmustine (BiCNU), Chlorambucil (Leukeran), Cisplatin, Cladribine (Leustat, LITAK), Clofarabine (Evoltra), Crisantaspase (Erwinase, asparaginase or L-asparaginase), Cyclophosphamide, Cytarabine, Dacarbazine (DTIC), Dactinomycin (Cosmegen Lyovac), Daunorubicin, Docetaxel (Taxotere), Doxorubicin (Adriamycin), Epirubicin (Pharmorubicin), Eribulin (Halaven), Etoposide (VP-16, Etopophos, Vepesid), Fludarabine (Fludara), Fluorouracil (5FU), Gemcitabine (Gemzar), Hydroxycarbamide (Hydrea, hydroxyurea), Idarubicin (Zavedos), Ifosfamide (Mitoxana), Irinotecan (Campto), Leucovorin (Folinic acid), Liposomal daunorubicin (DaunoXome), Liposomal doxorubicin (DaunoXome), Melphalan (Alkeran), Mercaptopurine (Puri-Nethol), Mesna (Uromitexan), Methotrexate (Maxtrex), Mitomycin (Mitomycin C Kyowa), Mitotane (Lysodren), Mitoxantrone, Oxaliplatin (Eloxatin), Paclitaxel (Taxol), Pemetrexed (Alimta), Pentostatin (Nipent), Procarbazine, Raltitrexed (Tomudex), Rasburicase (Fasturtec), Streptozocin (Zanosar), Temozolomide (Temodal), Thiotepa, Tioguanine (lanvis), Topotecan (Hycamtin), Trabectedin (Yondelis), Treosulfan, Vinblastine (Velbe), Vincristine (Oncovin), Vindesine (Eldisine), and Vinorelbine (Navelbine).

Immunotherapy includes treatment that helps the subject's immune system to target the cancer cells. Immunotherapies of use in embodiments of the invention include monoclonal antibodies such as those targeting CTLA4 or PD1, adoptive cell transfer which boosts the ability of T or NK cells to fight the cancer, cytokines such as interferons and interleukins, vaccines, and BCG.

Hormone therapy blocks the body's ability to produce hormones, or interferes with how the hormones behave. Hormone therapies of use in embodiments of the invention include estrogens and anti-estrogens, androgens and anti-androgens, progestins, gonadotropin-releasing hormone (GnRH) analogues and aromatase inhibitors.

Targeted therapy involves selecting drugs that specifically target changes that have occurred during the development of the specific cancer in the subject's body. Examples of targeted therapies that may be used in embodiments of the invention include small-molecule drugs and monoclonal antibodies. Preferably the targeted therapies in the embodiments of the invention will include one or more from the group consisting of Trastuzumab (Herceptin), ramucirumab (Cyramza), Vismodegib (Erivedge), sonidegib (Odomzo), Atezolizumab (Tecentriq), nivolumab (Opdivo), Bevacizumab (Avastin), Everolimus (Afinitor), tamoxifen (Nolvadex), toremifene (Fareston), fulvestrant (Faslodex), anastrozole (Arimidex), exemestane (Aromasin), lapatinib (Tykerb), letrozole (Femara), pertuzumab (Perjeta), ado-trastuzumab emtansine (Kadcyla), palbociclib (Ibrance), Cetuximab (Erbitux), panitumumab (Vectibix), ziv-aflibercept (Zaltrap), regorafenib (Stivarga), ramucirumab (Cyramza), Lanreotide acetate (Somatuline Depot), pembrolizumab (Keytruda), Imatinib mesylate (Gleevec), sunitinib (Sutent), regorafenib, Denosumab (Xgeva), Alitretinoin (Panretin), sorafenib (Nexavar), pazopanib (Votrient), temsirolimus (Torisel), axitinib (Inlyta), cabozantinib (Cabometyx), lenvatinib mesylate (Lenvima), crizotinib (Xalkori), erlotinib (Tarceva), gefitinib (Iressa), afatinib dimaleate (Gilotrif), ceritinib (LDK378/Zykadia), osimertinib (Tagrisso), necitumumab (Portrazza), alectinib (Alecensa), Ipilimumab (Yervoy), vemurafenib (Zelboraf), trametinib (Mekinist), dabrafenib (Tafinlar), cobimetinib (Cotellic), Bortezomib (Velcade), carfilzomib (Kyprolis), panobinostat (Farydak), daratumumab (Darzalex), ixazomib citrate (Ninlaro), elotuzumab (Empliciti), Dinutuximab (Unituxin), olaparib (Lynparza), rucaparib camsylate (Rubraca), niraparib (Zejula), talazoparib (Talzenna), enzalutamide (Xtandi), abiraterone acetate (Zytiga), radium 223 dichloride (Xofigo), Cabozantinib (Cometriq), and vandetanib (Caprelsa).

In some embodiments of the methods of treatment provided herein, the cancer is breast cancer and the treatment is one or more selected from the group consisting of surgery, radiation therapy, chemotherapy, hormonal therapy, immunotherapy, and targeted therapy. Preferably the chemotherapy involves treatment with one or more drugs selected from the group consisting of Abraxane (Abraxane), Bendamustine, (Levact), Bleomycin, Capecitabine (Xeloda), Carboplatin, Carmustine (BiCNU), Chlorambucil (Leukeran), Cisplatin, Cyclophosphamide (Cytoxan), Cytarabine, Dacarbazine (DTIC), Dactinomycin (Cosmegen Lyovac), Daunorubicin, Docetaxel (Taxotere), Doxorubicin (Adriamycin), Epirubicin (Pharmorubicin), Eribulin (Halaven), Etoposide (VP-16, Etopophos, Vepesid), Fluorouracil (5FU), Gemcitabine (Gemzar), Idarubicin (Zavedos), Ifosfamide (Mitoxana), Irinotecan (Campto), Liposomal doxorubicin (DaunoXome), Lobaplatin, Melphalan (Alkeran), Methotrexate (Maxtrex), Mitomycin (Mitomycin C), Mitoxantrone, Oxaliplatin (Eloxatin), Paclitaxel (Taxol), Pemetrexed (Alimta), Tegafur (Utefos), Temozolomide (Temodal), Thiotepa, Topotecan (Hycamtin), Trabectedin (Yondelis), Venetoclax (Venclexta), Vinblastine (Velbe), Vincristine (Oncovin), Vindesine (Eldisine), and Vinorelbine (Navelbine). The chemotherapy may involve treatment with one or more drugs selected from the group consisting of Capecitabine (Xeloda), Carboplatin (Paraplatin), Cisplatin (Platinol), Cyclophosphamide (Neosar), Docetaxel (Docefrez, Taxotere), Doxorubicin (Adriamycin), Pegylated liposomal doxorubicin (Doxil), Epirubicin (Ellence), Fluorouracil (5-FU, Adrucil), Gemcitabine (Gemzar), Methotrexate (multiple brand names), Paclitaxel (Taxol), Protein-bound paclitaxel (Abraxane), Vinorelbine (Navelbine), Eribulin (Halaven), mitoxantrone (Mitozantrone or Onkotrone), mitomycin C, Ixabepilone (Ixempra) and megestrol (Megace). Preferably the hormonal therapy involves treatment with one or more treatments selected from the group consisting of Tamoxifen, aromatase inhibitors (AIs) such as Anastrozole (Arimidex) and Exemestane (Aromasin), Letrozole (Femara), Fulvestrant (Faslodex), ovarian suppression or ablation such as using goserelin (Zoladex), megestrol acetate (Megace) and high-dose estradiol. Hormone therapies of use in embodiments of the invention include anti-estrogens e.g Raloxifene hydrochloride (Evista), medroxyprogesterone, Dromostanolone propionate (Masteril), luteinising hormone blockers e.g Goserelin (Zoladex), gonadotropin-releasing hormone (GnRH) analogues e.g Leuprolide acetate (Lucrin), Triptorelin pamoate (Decapeptyl SR), Buserelin acetate, and aromatase inhibitors e.g. formestane (Lentaron). Preferably the targeted therapy and/or immunotherapy involves treatment with one or more selected from the group consisting of palbociclib (Ibrance), Everolimus (Afinitor), Trastuzumab, Pertuzumab (Perjeta), Ado-trastuzumab emtansine or T-DM1(Kadcyla), Lapatinib (Tykerb), Bisphosphonates, and Denosumab (Xgeva). Immunotherapies of use in embodiments of the invention include monoclonal antibodies such as those targeting CTLA4 or PD1 or PDL1, adoptive cell transfer which boosts the ability of T cells to fight the cancer, cytokines such as interferons and interleukins, vaccines, immune system stimulators (CpG, Iquimod, 852A etc) such as engagement of Toll like receptors and live tumor targeted viruses or bacteria. Examples of targeted therapies that may be used in embodiments of the invention include small-molecule drugs and monoclonal antibodies. Preferably the targeted therapies in the embodiments of the invention will include one or more from the group consisting of Trastuzumab (Herceptin), ramucirumab (Cyramza), Vismodegib (Erivedge), sonidegib (Odomzo), Atezolizumab (Tecentriq), nivolumab (Opdivo), Bevacizumab (Avastin), Everolimus (Afinitor), tamoxifen (Nolvadex), afimoxifene, toremifene (Fareston), fulvestrant (Faslodex), anastrozole (Arimidex), exemestane (Aromasin), lapatinib (Tykerb), letrozole (Femara), pertuzumab (Perjeta), ado-trastuzumab emtansine (Kadcyla), palbociclib (Ibrance), ribociclib (Kisqali), abemaciclib (Verzenio), Alpelisib (BYL-719), Ipatasertib (RG-7440/GDC-0068), plinabulin (NPI-2358), tucidinostat (Chidamide), Cetuximab (Erbitux), panitumumab (Vectibix), ramucirumab (Cyramza), pembrolizumab (Keytruda), Denosumab (Xgeva), Margetuximab, Mogamulizumab (Poteligeo), sorafenib (Nexavar), pazopanib (Votrient), temsirolimus (Torisel), axitinib (Inlyta), cabozantinib (Cabometyx), lenvatinib mesylate (Lenvima), crizotinib (Xalkori), erlotinib (Tarceva), gefitinib (Iressa), afatinib dimaleate (Gilotrif), Ipilimumab (Yervoy), vemurafenib (Zelboraf), trametinib (Mekinist), neratinib (Nerlynx), pyrotinib (SHR-1258/HTI-1001), cobimetinib (Cotellic), Bortezomib (Velcade), panobinostat (Farydak), daratumumab (Darzalex), ixazomib citrate (Ninlaro), olaparib (Lynparza), rucaparib camsylate (Rubraca), niraparib (Zejula), talazoparib (Talzenna), enzalutamide (Xtandi), abiraterone acetate (Zytiga), Cabozantinib (Cometriq), and vandetanib (Caprelsa).

In some embodiments of the methods of treatment provided herein, the cancer is lung cancer and the treatment is one or more selected from the group consisting of surgery, radiotherapy, chemotherapy, photodynamic therapy (PDT), laser therapy, microwave or radiofrequency ablation, diathermy, and targeted therapies. Preferably the surgery includes wedge resection, segmentectomy, sleeve resection, lobectomy, bilobectomy, or pneumonectomy. Preferably the radiotherapy comprises external radiotherapy. Preferably the chemotherapy involves treatment with one or more drugs selected from the group consisting of Carboplatin (Paraplatin), Cisplatin (Platinol), Etoposide, Gemcitabine, Vinorelbine, Docetaxel (Taxotere), Paclitaxel (Taxol), or Pemetrexed. Preferably the targeted therapy involves treatment with one or more drugs selected from the group consisting of Bevacizumab (Avastin), pembrolizumab (Keytruda), Nivolumab (Opdivo), Ramucirumab (Cyramza), Erlotinib (Tarceva), Afatinib (Gilotrif), Gefitinib (Iressa), osimertinib (Tagrisso), Necitumumab (Portrazza), Crizotinib (Xalkori), Ceritinib (Zykadia), Alectinib (Alecensa), and Brigatinib (Alunbrig).

In some embodiments of the methods of treatment provided herein, the cancer is colon cancer and the treatment is one or more selected from the group consisting of surgery, radiotherapy, chemotherapy and targeted therapy. The surgery may be open or keyhole (laparoscopic), and may involve local resection or colectomy. Preferably the radiotherapy comprises brachytherapy and/or external radiotherapy. Preferably the chemotherapy involves treatment with one or more drugs selected from the group consisting of Folinic acid, fluorouracil and oxaliplatin (FOLFOX), Oxaliplatin and capecitabine (XELOX), Capecitabine (Xeloda), Fluorouracil, and Irinotecan (Campto). Preferably the targeted therapy involves treatment with one or more treatments selected from the group consisting of Bevacizumab (Avastin), Ramucirumab (Cyramza), Ziv-aflibercept (Zaltrap), Cetuximab (Erbitux), Panitumumab (Vectibix), and Regorafenib (Stivarga).

The biomarkers of the invention allow a diagnosis, prognosis and/or prediction to be made regarding the cancer. Therefore in some embodiments of the methods of the invention, particularly embodiments of the methods of treating cancer, this knowledge will be used to decide the best treatment option. For example, if the expression of the biomarkers in the cancer sample indicate that the cancer is more likely to recur, more likely to metastasize, and/or that the subject providing the sample is likely to have a reduced life expectancy, then a more aggressive treatment schedule may be used. For example, systemic therapy may be used before and/or after surgery, or treatments may be given for longer or at higher concentrations. Similarly, if the expression of the biomarkers in the cancer sample indicate a good outcome for cancer treatment, then the course of treatment followed may be less aggressive, for example it may be decided that there is no need to use adjuvant chemotherapy after surgery and/or any treatments may be given for less time or at lower concentrations, compared to treatments that would be given for a more aggressive cancer.

Binding Partners

Expression levels of the one or more biomarkers in a biological sample may be investigated using binding partners that bind or hybridize specifically to one or more target molecules for the one or more biomarkers, or a fragment thereof. In relation to the present invention the term ‘binding partners’ may include any ligands, which are capable of binding specifically to the relevant biomarker and/or nucleotide or peptide variants thereof with high affinity. Said ligands include, but are not limited to nucleic acids (DNA or RNA), proteins, peptides, antibodies, synthetic affinity probes, carbohydrates, lipids, artificial molecules or small organic molecules such as drugs and nanoparticles. In certain embodiments the binding partners may be selected from the group comprising: complementary nucleic acids; aptamers; antibodies or antibody fragments. In the case of detecting mRNAs and cDNAs, nucleic acids represent highly suitable binding partners. In the case of detecting proteins, antibodies and antibody fragments represent highly suitable binding partners.

In the context of the present invention, a binding partner specific to a biomarker should be taken as requiring that the binding partner should be capable of binding to at least one target molecule for such biomarker in a manner that can be distinguished from non-specific binding to molecules that are not target molecules for biomarkers. A suitable distinction may, for example, be based on distinguishable differences in the magnitude of such binding.

In preferred embodiments of the methods or devices of the invention, the target molecule for the biomarker is a nucleic acid, preferably an mRNA or cDNA molecule, and the binding partner is selected from the group consisting of complementary nucleic acids and aptamers.

Suitably the binding partner is a nucleic acid molecule (typically DNA, but it can be RNA) having a sequence which is complementary to the sequence of the relevant mRNA or cDNA against which is targeted. Such a nucleic acid is often referred to as a ‘probe’ (or a reporter or an oligo) and the complementary sequence to which it binds is often referred to as the ‘target’. Probe-target hybridization is usually detected and quantified by detection of fluorophore-, silver-, or chemiluminescence-labeled targets to determine relative abundance of nucleic acid sequences in the target.

Probes can be from 25 to 1000 nucleotides in length. However, lengths of 30 to 100 nucleotides are preferred, and probes of around 50 nucleotides in length are commonly used with great success in complete transcriptome analysis.

While the determination of suitable probes can be difficult, e.g. in very complex arrays, there are many commercial sources of complete transcriptome arrays available, and it is routine to develop bespoke arrays to detect any given set of specific mRNAs using publicly available sequence information. Commercial sources of microarrays for transcriptome analysis include Illumina and Affymetrix.

TABLE 1
Probe sequences and accession numbers for the biomarkers.

| HGNC | | SEQ | |
|---|---|---|---|
| Symbol | Probe Sequence (Illumina Human HT 12 V4 probes) | ID No. | Gene ID |
| SIGLEC1 | GAGACCACGCAGCTCATTGATCCTGATGCAGCCACATGTGAGACCTCAAC | 1 | ENSG00000088827 |
| CD163 | AGCCACAACAGGTCGCTCATCCCGTCAGTCATCCTTTATTGCAGTCGGGA | 2 | ENSG00000177575 |
| CCL8 | GTCATTGTTCTCCCTCCTACCTGTCTGTAGTGTTGTGGGGTCCTCCCATG | 3 | ENSG00000108700 |
| CD68 | GACGGGGTTTTCCTTGCTCCTGCCAGGATTAAAAGTCCATGAGTTTCTTG | 14 | ENSG00000129226 |

In one embodiment the probe sequences will comprise sequences selected from those listed in Table 1. However, nucleotide probe sequences may be designed to any sequence region of the biomarker transcripts (accession numbers listed in Table 1) or a variant thereof. Nucleotide probe sequences, for example, may include, but are not limited to those listed in Table 1. The person skilled in the art will appreciate that equally effective probes can be designed to different regions of the transcript than those targeted by the probes listed in Table 1, and that the effectiveness of the particular probes chosen will vary, amongst other things, according to the platform used to measure transcript abundance and the hybridization conditions employed. It will therefore be appreciated that probes targeting different regions of the transcript may also be used in accordance with the present invention.

In other suitable embodiments of the invention, the target molecule for the biomarker may be a protein, and the binding partner is selected from the group consisting of antibodies, antibody fragments and aptamers. Antibodies to SIGLEC1, CCL8 and CD163 are commercially available, as explained in the Materials and Methods part of the Experimental Results section below. Assays of particular interest for detecting protein biomarkers include mIHC, mass spectrometry CyTOP, and macrophage isolation and FACS.

Polynucleotides encoding any of the specific binding partners of target molecules for biomarkers of the invention recited above may be isolated and/or purified nucleic acid molecules and may be RNA or DNA molecules.

Throughout, the term “polynucleotide” as used herein refers to a deoxyribonucleotide or ribonucleotide polymer in single- or double-stranded form, or sense or anti-sense, and encompasses analogues of naturally occurring nucleotides that hybridize to nucleic acids in a manner similar to naturally occurring nucleotides. Such polynucleotides may be derived from Homo sapiens, or may be synthetic or may be derived from any other organism.

Commonly, polypeptide sequences and polynucleotides used as binding partners in the present invention may be isolated or purified. By “purified” is meant that they are substantially free from other cellular components or material, or culture medium. “Isolated” means that they may also be free of naturally occurring sequences which flank the native sequence, for example in the case of nucleic acid molecule, isolated may mean that it is free of 5′ and 3′ regulatory sequences.

In a preferred embodiment the nucleic acid is mRNA or cDNA. There are numerous suitable techniques known in the art for the quantitative measurement of RNA transcript levels in a given biological sample. These techniques include but are not limited to; “Northern” RNA blotting, Real Time Polymerase Chain Reaction (RTPCR), Quantitative Polymerase Chain Reaction (qPCR), digital PCR (dPCR), multiplex PCR, Reverse Transcription Quantitative Polymerase Chain Reaction (RT-qPCR), FISH, particularly RNA FISH, branched DNA signal amplification or by high-throughput analysis such as hybridization microarray, Next Generation Sequencing (NGS) or by direct mRNA quantification, for example by “Nanopore” sequencing. Alternatively, “tag based” technologies may be used, which include but are not limited to Serial Analysis of Gene Expression (SAGE). Suitable techniques also include nCounter™ systems of NanoString technologies™, zip coding, spatial transcriptomics, and targeted hybridization and sequencing. Sequencing may be carried out in situ and/or on one or more single cells. Commonly, the levels of biomarker mRNA transcript in a given biological sample may be determined by hybridization to specific complementary nucleotide probes on a hybridization microarray or “chip”, by Bead Array Microarray technology or by RNA-Seq where sequence data is matched to a reference genome or reference sequences.

In a preferred embodiment, where the nucleic acid is RNA, the present invention provides methods wherein the levels of biomarker transcript(s) will be determined by PCR. Preferably mRNA transcript abundance will be determined by qPCR, dPCR or multiplex PCR or single cell sequencing. More preferably, transcript abundance will be determined by multiplex-PCR. Nucleotide primer sequences may be designed to any sequence region of the biomarker transcripts (accession numbers listed in Table 1) or a variant thereof. The person skilled in the art will appreciate that equally effective primers can be designed to different regions of the transcript or cDNA of biomarkers listed in Table 1, and that the effectiveness of the particular primers chosen will vary, amongst other things, according to the platform used to measure transcript abundance, the biological sample and the hybridization conditions employed. It will therefore be appreciated that primers targeting different regions of the transcript may also be used in accordance with the present invention. However, the person skilled in the art will recognise that in designing appropriate primer sequences to detect biomarker expression, it is required that the primer sequences be capable of binding selectively and specifically to the cDNA sequences of biomarkers corresponding to the nucleotide accession numbers listed in Table 1 or fragments or variants thereof.

Many different techniques known in the art are suitable for detecting binding of the target molecule sequence and for high-throughput screening and analysis of protein interactions. According to the present invention, appropriate techniques include (either independently or in combination), but are not limited to; co-immunoprecipitation, bimolecular fluorescence complementation (BiFC), dual expression recombinase based (DERB) single vector system, affinity electrophoresis, pull-down assays, label transfer, yeast two-hybrid screens, phage display, in vivo crosslinking, tandem affinity purification (TAP), ChIP assays, chemical cross-linking followed by high mass MALDI mass spectrometry, strep-protein interaction experiment (SPINE), quantitative immunoprecipitation combined with knock-down (QUICK), proximity ligation assay (PLA), bio-layer interferometry, dual polarisation interferometry (DPI), static light scattering (SLS), dynamic light scattering (DLS), surface plasmon resonance (SPR), fluorescence correlation spectroscopy, fluorescence resonance energy transfer (FRET), isothermal titration calorimetry (ITC), microscale thermophoresis (MST), chromatin immunoprecipitation assay, electrophoretic mobility shift assay, pull-down assay, microplate capture and detection assay, reporter assay, RNase protection assay, FISH/ISH co-localization, microarrays, microsphere arrays or silicon nanowire (SiNW)-based detection and CODEX (CO-Detection by indexing) multiplexed imaging. Where biomarker protein levels are to be quantified, preferably the interactions between the binding partner and biomarker protein will be analyzed using antibodies with a fluorescent reporter attached.

In certain embodiments of the invention, the expression level of a particular biomarker may be detected by direct assessment of binding of the target molecule to its binding partner. Suitable examples of such methods in accordance with this embodiment of the invention may utilise techniques such as electro-impedance spectroscopy (EIS) to directly assess binding of binding partners (e.g. antibodies) to target molecules (e.g. biomarker proteins).

In certain embodiments of the present invention the binding partner may be an antibody, or antibody fragment, and the detection of the target molecules utilises an immunological method. In certain embodiments of the methods or devices, the immunological method may be an enzyme-linked immunosorbent assay (ELISA) or utilise a lateral flow device.

A method of the invention may further comprise quantification of the amount of the target molecules indicative of expression of the biomarkers that is present in the patient sample. Suitable methods of the invention, in which the amount of the target molecule present has been quantified, and the volume of the patient sample is known, may further comprise determination of the concentration of the target molecules present in the patient sample which may be used as the basis of a qualitative assessment of the patient's condition, which may, in turn, be used to suggest a suitable course of treatment for the patient.

Reporter Moieties

In preferred embodiments of the present invention the expression levels of the protein of the biomarker in a biological sample may be determined, for example by antibody probing for physical expression of the protein.

The expression levels of a particular biomarker may be detectable in a biological sample by a high-throughput screening method, for example, relying on detection of an optical signal, for instance using reporter moieties. For this purpose, it may be necessary for the specific binding partner to incorporate a tag, or be labelled with a removable tag, which permits detection of expression, or alternatively for a second binding partner to be used which includes a tag and is specific for the first binding partner (where the first binding partner is specific for the target molecule). Such a tag may be, for example, a fluorescence reporter molecule translationally-fused to the protein of interest (POI), e.g. Green Fluorescent Protein (GFP), Yellow Fluorescent Protein (YFP), Red Fluorescent Protein (RFP), Cyan Fluorescent Protein (CFP) or mCherry. Such a tag may provide a suitable marker for visualisation of biomarker expression since its expression can be simply and directly assayed by fluorescence measurement in vitro or on an array. Alternatively, it may be an enzyme which can be used to generate an optical signal. Tags used for detection of expression may also be antigen peptide tags. Similarly, reporter moieties may be selected from the group consisting of fluorophores; chromogenic substrates; and chromogenic enzymes. Other kinds of label may be used to mark a nucleic acid binding partner including organic dye molecules, radiolabels and spin labels which may be small molecules.

Preferably, the levels of a biomarker or several biomarkers will be quantified by measuring the specific hybridization of a complementary nucleotide probe to the target molecule for the biomarker of interest under high-stringency or very high-stringency conditions.

Preferably, probe-target molecule hybridization will be detected and quantified by detection of fluorophore-, silver-, or chemiluminescence-labelled probes to determine relative abundance of biomarker nucleic acid sequences in the sample. Alternatively, levels of biomarker mRNA transcript abundance can be determined directly by RNA sequencing or nanopore sequencing technologies.

The methods or devices of the invention may make use of target molecules selected from the group consisting of: the biomarker protein; and nucleic acid encoding the biomarker protein. Where the target molecule is the biomarker protein, it is preferred that the binding partner is an antibody or antibody fragment and immunofluorescence is used to detect binding; methods of detecting bound antibodies and antibody fragments are well known in the art and may include the use of a tag attached to the antibodies or fragments themselves, and/or the use of additional antibodies that have such a detectable, e.g. fluorescent, tag and are specific for the primary antibody or fragment.

Nucleotides and Hybridization Conditions

Throughout, the term “polynucleotide” as used herein refers to a deoxyribonucleotide or ribonucleotide polymer in single- or double-stranded form, or sense or anti-sense, and encompasses analogues of naturally occurring nucleotides that hybridize to nucleic acids in a manner similar to naturally occurring nucleotides.

Exemplary probe sequences are provided in Table 1, although it will be appreciated that minor variations in these sequences may work. The person skilled in the art would regard it as routine to design nucleotide probe sequences to any sequence region of the biomarker transcripts (accession numbers listed in Table 1) or a variant thereof. This is also the case with nucleotide primers used where detection of expression levels is determined by PCR-based technology. Nucleotide probe sequences, for example, may include, but are not limited to those listed in Table 1. The person skilled in the art will appreciate that equally effective (and in some cases more beneficial) probes can be designed to different regions of the transcript than those targeted by the probes listed in Table 1, and that the effectiveness of the particular probes chosen will vary, amongst other things, according to the platform used to measure transcript abundance and the hybridization conditions employed. It will therefore be appreciated that probes targeting different regions of the transcript may also be used in accordance with the present invention.

Of course the person skilled in the art will recognise that in designing appropriate probe sequences to detect biomarker expression, it is required that the probe sequences be capable of binding selectively and specifically to the transcripts or cDNA sequences of biomarkers corresponding to the nucleotide accession numbers listed in Table 1 or fragments or variants thereof. The probe sequence will therefore be hybridizable to that nucleotide sequence, preferably under stringent conditions, more preferably very high stringency conditions. The term “stringent conditions” may be understood to describe a set of conditions for hybridization and washing and a variety of stringent hybridization conditions will be familiar to the skilled reader. Hybridization of a nucleic acid molecule occurs when two complementary nucleic acid molecules undergo an amount of hydrogen bonding to each other known as Watson-Crick base pairing. The stringency of hybridization can vary according to the environmental (i.e. chemical/physical/biological) conditions surrounding the nucleic acids, temperature, the nature of the hybridization method, and the composition and length of the nucleic acid molecules used. Calculations regarding hybridization conditions required for attaining particular degrees of stringency are discussed in Sambrook et al. (2001, Molecular Cloning: A Laboratory Manual, Cold Spring Harbor Laboratory Press, Cold Spring Harbor, N.Y.); and Tijssen (1993, Laboratory Techniques in Biochemistry and Molecular Biology—Hybridization with Nucleic Acid Probes Part I, Chapter 2, Elsevier, N.Y.). The Tm is the temperature at which 50% of a given strand of a nucleic acid molecule is hybridized to its complementary strand.

In any of the references herein to hybridization conditions, the following are exemplary and not limiting:

Very High Stringency (allows sequences that share at least 90% identity to hybridize)

Hybridization: 5×SSC at 65° C. for 16 hours

Wash twice: 2×SSC at room temperature (RT) for 15 minutes each

Wash twice: 0.5×SSC at 65° C. for 20 minutes each

High Stringency (allows sequences that share at least 80% identity to hybridize)

Hybridization: 5×-6×SSC at 65° C.-70° C. for 16-20 hours

Wash twice: 2×SSC at RT for 5-20 minutes each

Wash twice: 1×SSC at 55° C.-70° C. for 30 minutes each

Low Stringency (allows sequences that share at least 50% identity to hybridize)

Hybridization: 6×SSC at RT to 55° C. for 16-20 hours

Wash at least twice: 2×-3×SSC at RT to 55° C. for 20-30 minutes each.

Diagnostic Devices and Kits

The invention also provides an assay device for use in the above methods, the device comprising: a) a loading area for receipt of a biological sample; b) binding partners specific for target molecules representative of expression of SIGLEC1 and/or CCL8, and optionally CCL8 and/or CD68; and c) detection means to detect the levels of said target molecules present in the sample. The invention provides an assay device for use in embodiments of the above methods, the device comprising: a) a loading area for receipt of a biological sample; b) binding partners specific for target molecules representative of expression of CCL8, and optionally SIGLEC1 and/or CD163 and/or CD68; and c) detection means to detect the levels of said target molecules present in the sample.

Suitably the device comprises specific binding partners for amplifying the target molecules of the biomarkers. Suitable binding partners and associated reporter moieties for use in the devices and kits of the invention are described above. A variety of suitable PCR amplification-based technologies are well known in the art.

The binding partners are preferably nucleic acid primers adapted to bind specifically to the mRNA or cDNA transcripts of one of the biomarkers, or one or more labelled antibodies that binds to one of the biomarker proteins, as discussed above. Suitably, the kit may comprise a combination of nucleic acid primers and antibodies, for example nucleic acid primers may be provided in the kit for analyzing the levels of CCL8 and optionally SIGLEC1, whilst antibodies may be provided for analyzing the levels of CD163 and/or CD68, and optionally SIGLEC1.

The detection means suitably comprises means to detect a signal from a reporter moiety, e.g. a reporter moiety as discussed above.

The device is adapted to detect and quantify the levels of said biomarkers present in the biological sample.

The invention provides kits for use in the above methods, the kits comprising binding partners capable of binding to target molecules representative of expression of SIGLEC1 and/or CCL8, and optionally CCL8 and/or CD68. Preferably the kits further comprise indicators capable of indicating when said binding occurs.

Preferably the kits and devices comprise binding partners capable of binding to target molecules representative of expression of at least two of the biomarkers, for example SIGLEC1 and CCL8, or SIGLEC1 and CD163, or SIGLEC1 and CD68, or CCL8 and CD163, or CCL8 and CD68, and preferably three of the biomarkers, e.g. SIGLEC1, CCL8 and CD163 or SIGLEC1, CCL8 and CD68. Most preferably, the kits and devices comprise binding partners capable of binding to target molecules representative of expression of all four of the biomarkers, i.e. SIGLEC1, CCL8, CD163 and CD68.

PCR applications are routine in the art and the skilled person will be able to select appropriate polymerases, buffers, reporter moieties and reaction conditions.

The binding partners are preferably nucleic acid primers adapted to bind specifically to the mRNA or cDNA transcripts of biomarkers, as discussed above. The nucleic acid primers may be provided in a lyophilized or reconstituted form, or may be provided as a set of nucleotide sequences. In one embodiment, the primers are provided in a microplate format, where each primer set occupies a well (or multiple wells, as in the case of replicates) in the microplate. The microplate may further comprise primers sufficient for the detection of one or more housekeeping genes as a positive control. The kit may further comprise reagents and instructions sufficient for the amplification of expression products from the biomarkers.

As well as the binding partners for the target molecules of the biomarkers, the devices and kits may further comprise binding partners capable of binding to target molecules representative of expression of additional genes. For example, such genes may be “housekeeping genes”, which can act as a positive control and/or to normalize expression across samples, and/or such genes may give an indication of the concentration of the monocyte population within the biological sample.

Preferably said devices and kits provide binding partners capable of binding to target molecules representative of expression of less than 20, 15, 10, 7, 6, 5, or 4 genes, including the biomarkers and any housekeeping or other control genes.

The kit can optionally comprise instructions for carrying out the analysis required for the methods of the invention.

Methods of Screening

The inventors have identified SIGLEC1, CD163, CD68 and CCL8 as key proteins expressed by TAMs, and increased expression of these proteins is associated with more aggressive disease, showing more metastasis, a greater likelihood of recurrence and generally a poorer prognosis. The identification of these proteins in this context, and their link with cancer cell invasion, motility and a poorer prognosis, are consistent with the concept that TAMs in the tumor microenvironment promote malignancy. Thus by identifying these key proteins, the inventors have identified important targets for therapeutics that can be used to treat cancer and so reduce malignancy. Thus the invention also provides a method of identifying one or more molecules for use in treating cancer. The method may comprise identifying a molecule that binds SIGLEC1, or CD163, or CCL8 or a CCL8 receptor. Thus the method may comprise the steps of a) preparing a candidate molecule, b) contacting a cell that expresses SIGLEC1, CD163, CCL8, and/or a CCL8 receptor, with the candidate molecule, and c) determining whether said candidate molecule binds the SIGLEC1, CD163, CCL8 and/or CCL8 receptor and affects its activity. Alternatively, the method may comprise identifying a molecule that interferes with expression of SIGLEC1, or CD163, or CCL8 or a CCL8 receptor. Thus, the method may comprise the steps of a) preparing a candidate molecule, b) contacting a cell that expresses SIGLEC1, CD163, CCL8, and/or a CCL8 receptor, with the candidate molecule, and c) determining whether said candidate molecule interferes with either transcription or translation of the SIGLEC1, CD163, CCL8 and/or CCL8 receptor and thereby affects its expression. A candidate molecule that inhibits the activity, and/or downregulates the expression, of the SIGLEC1, CD163, or CCL8 protein or a CCL8 receptor may be identified as for use in treating cancer.

Preferably the CCL8 receptor will be CCR1 (C-C motif chemokine receptor 1; ENSG00000163823), CCR2 (C-C motif chemokine receptor 2; ENSG00000121807), CCR3 (C-C motif chemokine receptor 3 gene; ENSG00000183625.14), CCR5 (C-C motif chemokine receptor 5 (gene/pseudogene); ENSG00000160791) or CCR8 (C-C motif chemokine receptor 8; ENSG00000179934). It is particularly preferred that the receptor is CCR8. Preferably the method is for identifying an antagonist of SIGLEC1.

Preferably the cell used in the method of identifying a molecule will be an induced Pluripotent stem cell (iPS) derived macrophage conditioned by tumor cell conditioned media, or the cell may be from a mouse model of cancer. Activities include the ability of a cell to immunosuppress an immune response through blocking action of cytotoxic cells such as T cells or NK cells, to block the migration or invasion of tumor cells in response to the candidate molecule, to inhibit angiogenesis in the tumor or its metastatic site, to increase the viability of the tumor cells, to increase their extravasation and survival and spread in a metastatic site, and to inhibit expression of a target molecule disclosed herein, for example a molecule that binds and inhibits CCL8 or a CCL8 receptor activity or expression may cause down-regulation of SIGLEC1 expression and/or inhibit the invasiveness of tumor cells. A molecule that binds and inhibits SIGLEC1 activity or expression may inhibit immunosuppression/regulation, and may promote adherence of macrophages to tumor cells or matrix. Tumor cell invasiveness, for example, may be assessed using in vitro assays known in the art, such as a scratch assay or collagen invasion assay. The activity inhibited may be antigen specific T-cell suppression. In addition said inhibitory molecule may block the pro-tumoral phenotype of macrophages and convert it to one that is anti-tumoral.

As mentioned above and as explained further below, the activities assessed for downregulation in the methods of identifying a molecule may include macrophage suppression of T or NK cell killing, macrophage promotion of tumor cell invasion, macrophage promotion of trans-endothelial migration and survival, macrophage promotion of angiogenesis, macrophage promotion of cancer stem cell survival, and mouse or other animal (monkey) models of cancer with and without therapeutics directed to targets and/or in combination with check-point inhibitors.

The method of identifying a molecule may comprise identifying an antibody that binds and then adapting the structure of the antibody in order to minimize unwanted immunological responses, for example using humanization and/or deimmunization of the antibody.

Thus the invention provides therapeutic molecules for use in treating cancer that can be found using these methods, for example the therapeutic molecule may be for use in inhibiting metastasis and/or for use in inhibiting recurrence. Preferably the molecule will be an antibody, further preferably a monoclonal antibody. Alternatively, preferably the molecule will be a nucleic acid molecule able to downregulate biomarker expression, for example through RNA interference; this is particularly preferred in the context of downregulating CCL8 expression. Preferably, a molecule targeting the CCL8 receptor will be a small molecule inhibitor.

The therapeutic molecule, for example monoclonal antibody, may be provided with pharmaceutical excipients for administration to a patient. Preferably the therapeutic molecule will be administered in a method of treatment according to the invention as described above. Therapeutic molecules may be delivered to cells, for example cancer cells, for treatment using vehicles known in the art. For example inhibitory RNAs may be delivered in a variety of ways, including using nanoparticles, viruses or macrophages themselves.

### BRIEF DESCRIPTION OF THE FIGURES

The invention will now be described in detail with reference to the accompanying drawings, in which:

FIG. 1. Sorting strategy for tissue macrophages and TAMs and analysis of CD163 expression.

A)Gating strategy for tissue macrophages and TAMs; macrophages were defined as CD45⁺CD3/56/19⁻CD11b⁺CD14⁺CD163⁺.

B) (Left) Representative histogram of macrophage CD163 expression; dark grey histogram=CD163 staining, light grey histogram=Fluorescence minus one (FMO) control;

(Right) Representative histogram of macrophage CD163 expression in Br-MR (n=5) compared to Br-TAM (n=5). Data are expressed as Geometric Mean (Mean±SEM).

FIG. 2. TAMs from breast and endometrial cancers exhibit cancer-specific transcriptional profiles.

A) PCA plot of N=13,668 genes expressed in breast tissue resident macrophages (Br-RM, N=4) and breast cancer TAMs (Br-TAM, N=4). B) Hierarchical clustering of all DEGs between Br-RM and Br-TAM. Expression values are Z score-transformed. Samples were clustered using complete linkage and Euclidean distance. C) Gene ontology analysis of DEGs between Br-TAM and Br-RM (five GO groups at bottom of chart=down-regulated genes, six GO groups at top of chart=upregulated genes). D) Bar plot of selected DEGs in Br-TAM (FDR<=0.05). E) PCA plot of N=13,739 genes expressed in endometrial tissue resident macrophages (En-RM, N=5) from healthy individuals and endometrial cancer TAMs (En-TAM, N=9). F) Hierarchical clustering of all DEGs between En-RM and En-TAM. Expression values are Z score-transformed. Samples were clustered using complete linkage and Euclidean distance. G) Gene ontology analysis of DEGs between En-TAM and En-RM (nine GO groups at bottom of chart=down-regulated genes, three GO groups at top of chart=upregulated genes).H) Bar plot of selected DEGs in En-TAM (FDR<=0.05).

FIG. 3. Comparison of TAMs and resident macrophages.

A) (Left) PCA plot of N=14,229 expressed genes in Br-TAM (n=4) and En-TAM (n=9). (Right) Hierarchical clustering of all DEGs between Br-TAM and En-TAM. Expression values are Z score-transformed. Samples were clustered using complete linkage and Euclidean distance.

B) (Left) PCA plot of N=13,907 expressed genes in Br-RM (n=4) and En-RM (n=5). (Right) Hierarchical clustering of all DEGs between Br-RM against En-RM. Expression values are Z score-transformed. Samples were clustered using complete linkage and Euclidean distance.

C) Enrichment analysis of M1-like (left) and M2-like (right) macrophage signature [3] in Br-TAM. Black bars represent the position of M1-like or M2-like genes in the ranked list of Br-TAM expressed genes together with the running enrichment score.

D) Enrichment analysis of M1-like (left) and M2-like (right) macrophage signature in En-TAM. Black bars represent the position of M1-like or M2-like genes in the ranked list of Br-TAM expressed genes together with the running enrichment score.

FIG. 4. Data filtering and transmembrane receptor expression on TAMs.

A) Venn diagram showing comparison between the BRTAMs dataset, the Finak et al dataset (human tumor stroma, [4]) and the Ojalvo et al dataset (mouse mammary gland TAMs, [5]). 64 genes were commonly upregulated in all three datasets. The upper or top number in each pair of numbers is the number of up-regulated genes, and the bottom number in each pair of numbers is the number of down-regulated genes.

B) List of genes encoding for transmembrane receptors commonly upregulated in all 3 datasets; SIGLEC1 is the top upregulated gene.

FIG. 5. Breast TAM transcriptomes are associated with clinical outcomes and reveal new TAM-specific markers.

A) Scatterplot showing Pearson's correlation between CD163 and SIGLEC1 expression in the METABRIC cohort. The line indicates local regression (LOESS) fit. B) Disease-specific survival according to the mRNA level of SIGLEC1 in the METABRIC cohort. C) Expression of SIGLEC1 mRNA in Br-RM (n=4) and Br-TAM (n=6). D) SIGLEC1 expression in the Finak et al. dataset [4] (Left graph) and the Karnoub et al. dataset [6] (right graph). Expression calculated from the median centered normalized values. p values were estimated using a Wilcoxon rank sum test. Boxplots depict the first and third quartiles, with the median shown as a solid line inside the box and whiskers extending to 1.5 interquartile range from first and third quartiles. Data points beyond the limit of lines represent outliers (black dots) E) CD163 and SIGLEC1 immunofluorescent (IF) staining (n=5). Stains from cancer (top) and benign sample (bottom) are shown representative of n=12 independent tumors analyzed. Single channels and merge are shown. Inset representing a double positive SIGLEC1 and CD163 macrophage (top) and a single positive CD163 macrophage (bottom). Scale bars=50 μm and inset=5 μm. F) Quantification of CD163⁺ (left), SIGLEC1⁺ (right), and CD163⁺ and SIGLEC1⁺ (bottom) cells per mm² of tissue in benign (n=4) and breast cancer samples (n=8). Boxplots depict the first and third quartiles, with the median shown as a solid line inside the box and whiskers extending to 1.5 interquartile range from first and third quartiles (C) Horizontal bars represent the mean of the individual values±SD; (B) p value is based on Wald test, (C) Student's t-test, (F) Two-way ANOVA; *p<0.01, **p<0.001, ***p<0.0001, ****p<0.00001.

FIG. 6. Flow diagram showing the acquisition and analysis pipeline for cell quantification of immunofluorescently stained breast cancer tissues.

A) Images of 40×-scanned slides were processed in Tissue Studio for nuclear detection and cell simulation. Nuclear/cell quality control and manual sampling of cell classes were conducted in Developer XD and a 19-dimensional feature space optimized from 49 metrics and ranked by importance to Euclidean distance separation. Initial classification of tissues was refined 10 or more times using cycles of classification-correction-reclassification. This process was repeated for additional channels, then entire batches of scanned slides processed together and cell classification data exported. Final extraction, compilation, and statistics of cell numbers were conducted using Mathematica and GraphPad Prism as described in materials and methods. B) CD163 and SIGLEC1 immunofluorescent staining on breast cancer tissue samples (n=5 each, Bars=50 μm, inset=5 μm, 3 representative samples are shown). Enlargement of the selected area showing a representative SIGLEC1⁺ CD163⁺ macrophage.

FIG. 7. SIGLEC1 survival analysis in publicly available datasets.

A) Disease-specific survival according to the mRNA level of SIGLEC1/CD163 in GSE31210 lung cancer cohort.

B) Disease-specific survival according to the mRNA level of SIGLEC1/CD163 in GSE1433 (colon1) and GSE17536 (colon2) colon cancer cohorts.

FIG. 8. Expression of SIGLEC1 in Br-TAM and other immune cell types.

A) SIGLEC1 expression in Br-TAM samples (top), in CD3⁺/19⁺/56⁺ cells (middle) and CD45⁻ cells (bottom) from breast tumors. FMO controls (left), SIGLEC1 staining (right). Representative plot of 3 independent experiments is shown.

B) Quantification of SIGLEC1⁺ cells in Br-TAM, CD3⁺/19⁺/56⁺ cells and CD45⁻ cells from breast tumors (n=3). Data are depicted as % of SIGLEC1⁺ cells (top) and GEO Mean values (bottom).

C) SIGLEC1 expression in classical and non-classical Mo and breast cancer TEMo (square gate based on FMO control). Representative plot of 3 independent experiments is shown.

D) Quantification of % of SIGLEC1⁺ cells in Mo and TEMo (n=3). Data are depicted as % of SIGLEC1⁺ cells.

E) SIGLEC1 expression in blood circulating granulocytes from healthy donors (CTR) and breast cancer patients (gated on live CD45⁺ SSC high cells, square gate based on FMO control). Representative plot of 3 independent experiments is shown.

F) Quantification of % of SIGLEC1⁺ cells in blood circulating granulocytes from healthy donors (CTR) and breast cancer patients (n=3).

*p<0.01, **p<0.001; (B) One-way ANOVA, (D,F) Student's t-test, (B, D, F) Data depicted as Mean±SEM.

FIG. 9. Multiplex immunohistochemistry analysis of different breast cancer subtypes.

A) Tissue biopsies from 13 breast cancer patients and three prophylactic mastectomy/mammoplasty (PM) procedures were assessed using quantitative multiplex immunohistochemistry. Cumulative cell populations (indicted on right) from total tissue areas were normalized to total cell number.

B) Representative micrographs reflecting pseudo-colored images following multiplex IHC of cell populations across breast cancer subtypes as indicated. Boxed insets are depicted at higher magnification in corresponding columns. Scale bars as indicated.

C) A heatmap of each cell population as a percent of total cells is shown with a dendrogram of unsupervised hierarchical clustering, scaled by row and using correlation as a distance measure, and average as a clustering method. Each column represents an independent tumor according to sub-type. (Lum: luminal) and prophylactic mastectomy/mammoplasty (PM) samples.

FIG. 10. Expression of SIGLEC1 in macrophages after stimulation with cancer cell conditioned medium or cytokines.

A) SIGLEC1 mRNA expression in primary MDM stimulated for 24 h with PBS (black bar) normalized as 1, MDA-MB-231 conditioned medium (CM) (middle bar) or MDA-MB-468 CM (right bar). Data are depicted as fold change vs CTR (n=3, Mean±SEM).

B) SIGLEC1 mRNA expression in PMA-treated THP1 cells stimulated for 24 h with PBS (black bar) normalized as 1, MDA-MB-231 CM (middle bar) or MDA-MB-468 CM (right bar). Data are depicted as fold change vs CTR (n=3, Mean±SEM).

C) Flow cytometric analysis of SIGLEC1 expression in iPSDM cells stimulated with MDA-MB-231 CM. (Left panel) representative histogram showing SIGLEC1 expression levels in unstained (light grey histogram), PBS (black histogram) and MDA-MB-231 CM treated samples (dark grey histogram). (Right panel) SIGLEC1 expression (Geometric mean expressed as fold change vs CTR) in PBS (black bar) and MDA-MB-231 CM treated samples (grey bar). (n=3, Mean±SEM).

D) Flow cytometric analysis of SIGLEC1 expression in iPSDM cells stimulated with MDA-MB-468 CM. (Left panel) representative histogram showing SIGLEC1 expression levels in unstained (light grey histogram), PBS (black histogram) and MDA-MB-468 CM treated samples (dark grey histogram). (Right panel) SIGLEC1 expression (Geometric mean expressed as fold change vs CTR) in PBS (black bar) and MDA-MB-468 CM treated samples (grey bar). (n=3, Mean±SEM).

E) SIGLEC1 mRNA expression in PMA-treated THP1 cells stimulated for times shown with pro-inflammatory cytokines as indicated. LPS acts as a positive pro-inflammatory signal control. Solid black line indicates SIGLEC1 expression in treated samples, dotted black line indicates SIGLEC1 expression in control PBS-treated samples. Data are depicted as fold change vs CTR, Mean±SEM (n=3).

F) SIGLEC1 mRNA expression in PMA-treated THP1 cells stimulated for times shown with anti-inflammatory cytokines as indicated. Solid black line indicates SIGLEC1 expression in cytokine-treated samples, dotted black line indicates SIGLEC1 expression in PBS-treated samples. Data are depicted as fold change vs CTR, Mean±SEM (N=3).

FIG. 11. TNFα expression in TAMs

A) TNFα levels in supernatants of iPSDM incubated for 24 h with PBS, PBS plus isotype control and PBS plus anti-TNFα antibody (3^{rd} 4^{th} and 5^{th} bars from the left). Same conditions are shown for MDA-MB-231 (6^{th}, 7^{th} and 8^{th} bars from the left) and MDA-MB-468 CM (the three bars nearest the right of the chart) (n=3). Results are expressed as pg/ml. B) Expression of TNFA mRNA in Br-RM (N=4) and Br-TAM (n=6). C) TNFα protein levels in MDM supernatants incubated for 24 h with MDA-MB-231 and MDA-MB-468 conditioned medium (CM). Results are expressed as OD at 450 nm (n=3). D) SIGLEC1 mRNA expression in iPSDM stimulated for 24 h with MDA-MB-231 CM normalized as 1 (left bar), MDA-MB-231 CM+ TNFα neutralizing antibody (middle bar) and MDA-MB-231 CM+ isotype control antibody (right bar) (n=3). E) SIGLEC1 mRNA expression in iPSDM stimulated for 24 h with MDA-MB-468 CM normalized as 1 (left bar), MDA-MB-468 CM+ TNFα neutralizing antibody (middle bar) and MDA-MB-468 CM+ isotype control antibody (right bar). (n=3). *p<0.01, **p<0.001, ***p<0.0001, ****p<0.00001; (Data on expression are presented as Mean±SEM, Data are depicted as fold change vs control.)

FIG. 12. TAMs and cancer cells engage in cytokine feedback loops to support CCL8 and SIGLEC1 expression in breast cancer TAMs.

(A and B) Volcano plot showing genes whose expression was significantly (Log₂FC+/−1, p<0.05) deregulated in PMA-THP1 cells after incubation with MDA-MB-231 (A) or MDA-MB-468 (B) CM for 24 hr (n=3 each). (C) Venn diagram of commonly upregulated transcripts between MDA-MB-231 (left circle) and MDA-MB-468 (right circle) treated THP1 cells. (D) Selection of pro-inflammatory genes commonly upregulated in Br-TAM (n=4) (from RNA-seq analysis) and PMA-THP1 (n=3) (qPCR). (E) Scatterplot showing Pearson's correlation between CD163 and CCL8 expression in the METABRIC cohort. The line indicates local regression (LOESS) fit. (F) Disease-specific survival according to the mRNA level of CCL8 in the METABRIC cohort. (G) CCL8 mRNA expression in Br-RM (n=4) and Br-TAM (n=7). Data are expressed as fold change vs Br-RM. (H) CCL8 levels in CM from MDA-MB-231, MDA-MB-468, MDM, and MDM incubated for 24 hr with the two cancer cell CM, respectively. (n=3). (I) IF and FISH for CCL8 mRNA (top) or a DapB-control RNA (bottom) in breast cancer samples. All scale bars are 10 μm. (n=3) Inset representing a SIGLEC1⁺ CD163⁺ macrophage expressing CCL8 mRNA (top) or DapB-control mRNA (bottom). XY, XZ and YZ projections are shown (right panels)

(J and K) CCL8 mRNA expression in iPSDM stimulated for 24 hr with MDA-MB-231 CM normalized as 1 (CTR), MDA-MB-231 CM+ TNFα neutralizing antibody and MDA-MB-231 CM+ isotype control antibody (J) or with MDA-MB-468 CM normalized as 1 (CTR), MDA-MB-468 CM+ TNFα neutralizing and MDA-MB-468 CM+ isotype control antibody (K) (n=3 each). (L and M) CSF1 levels (L) and TNFα and IL1β levels (M) in supernatants from unstimulated MDA-MB-231 or MDA-MB-468 (CTR), and MDA-MB-231 or MDA-MB-468 incubated for 24 hr with 10 or 20 ng/ml (or 20 ng/ml for CSF1) of rCCL8, (n=3 each). (N) In vitro scratch assay of untreated MDA-MB-231 or treated with CCL8 or CCL2 for the indicated period of time, line=cell culture margins (n=4). All scale bars are 500 μm. (O) Quantification of in vitro scratch assay covered by MDA-MB-231 after 24 hr (calculated as area covered at 24 hr −1 hr) in untreated (CTR), CCL8- and CCL2-treated cells. Same symbols represent mean of technical replicates (n=4). (P) THP1 chemotaxis assay for CCL2 and CCL8. Cells were incubated with medium alone (CTR) or with 20 ng/ml of rCCL2 or rCCL8. Results shown as fold change vs CTR at 72 hr (n=3). (H, J, K, M, P) data depicted as Mean±SEM; (G, L) Horizontal bars represent the mean of the individual values±SD; (O) Horizontal bars represent the mean of the individual values. (F) p value is based on Wald test, (G, L) Student's t-test, (H, J, K, M, P) One-way ANOVA, (O) Two-way ANOVA, *p<0.01, **p<0.001, ***p<0.0001.

FIG. 13. Expression of CCL8 in macrophages after stimulation with cytokines

A) CCL8 mRNA expression in PMA-treated THP1 cells stimulated for 24 h with PBS (CTR; left bar), MDA-MB-231 CM (middle bar) or MDA-MB-468 CM (right bar). Data are depicted as fold change vs CTR (n=3, Mean±SEM).

B) CCL8 mRNA expression in primary MDM stimulated for 24 h with PBS (CTR; left bar), MDA-MB-231 CM (middle bar) or MDA-MB-468 CM (right bar). Data are depicted as fold change vs CTR (n=3 Mean±SEM).

C) CCL8 mRNA expression in iPSDM stimulated for 24 h with PBS (CTR; left bar), MDA-MB-231 CM (middle bar) or MDA-MB-468 CM (right bar). Data are depicted as fold change vs CTR (n=3, Mean±SEM).

D) ELISA for CCL8 in serum of healthy donors (black circles, N=21) and breast cancer patients (grey squares, N=38). Data are depicted as pg/ml (Mean±SEM).

E) CCL8 mRNA expression in PMA-treated THP1 cells stimulated for times shown with pro-inflammatory cytokines as shown. Solid line cytokine-treated samples, dotted line PBS-treated samples; Data are depicted as fold change vs CTR (n=3, Mean±SEM).

F) CCL8 mRNA expression in PMA-treated THP1 cells stimulated for times shown with anti-inflammatory cytokines as indicated. Solid line cytokine-treated samples, dotted line PBS-treated samples. Data are depicted as fold change vs CTR (n=3, Mean±SEM).

FIG. 14. Cancer cell line expression of CCL8 Receptors.

A) Representative histograms of CCR1, CCR2, CCR3, CCR5 and CCR8 expression in MDA-MB-231 (top) and MDA-MB-468 (bottom) cells. Histogram peaks to the left indicate unstained samples. Representative plot of 3 independent experiments is shown.

B) Percentage of CCR1, CCR2, CCR3, CCR5 and CCR8 positive cells in total MDA-MB-231 (left panel) and MDA-MB-468 cells (right panel), (n=3).

C) MDA-MB-231 (left) and MDA-MB-468 (right) proliferation assay in the presence of PBS (No treatment), 0.1 ng/ml, 1 ng/ml and 10 ng/ml of CCL8 from 0-80 hr. No statistical differences between treatments and controls (n=3).

(B, C) Data depicted as Mean±SEM

FIG. 15. Breast cancer qPCR array on cancer cells stimulated with rCCL8 and macrophage conditioned medium.

A) Volcano plot showing genes whose expression is significantly (Log₂FC +/−2, p<0.05) down-regulated (grey dots to the left of the first dotted line) and up-regulated (grey dots to the right of the second dotted line) in MDA-MB-231 cells after incubation with 1 ng/ml of rCCL8 for 16 h (n=3).

B) Volcano plot showing genes whose expression is significantly (Log₂FC +/−2, p<0.05) down-regulated (grey dots to the left of the first dotted line) and up-regulated (grey dots to the right of the second dotted line) in MDA-MB-468 cells after incubation with 1 ng/ml of rCCL8 for 16 h (n=3).

C) Venn diagram of commonly up-regulated genes between MDA-MB-231 (left circle) and MDA-MB-468 (right circle) after rCCL8 treatment. Expression of 6 genes was commonly up-regulated.

D) mRNA expression of 6 commonly up-regulated genes in MDA-MB-231 or MDA-MB-468 after CCL8 stimulation; dotted black line represents normalized expression level in untreated control samples. Data are depicted as fold change vs CTR (n=3, Mean±SEM).

E) Volcano plot showing genes whose expression is significantly (Log₂FC +/−2, p<0.05) down-regulated (grey dots below the dotted lines) and up-regulated (grey dots above the dotted lines) in MDA-MB-231 cells after incubation with condition medium (CM) primed MDM supernatant for 16 h (N=3).

F) Volcano plot showing genes whose expression is significantly (Log₂FC +/−2, p<0.05) down-regulated (grey dots below the dotted lines) and up-regulated (grey dots above the dotted lines) in MDA-MB-468 cells after incubation with CM primed MDM supernatant for 16 h (N=3).

G) Venn diagram of commonly upregulated genes between MDA-MB-231 (left circle) and MDA-MB-468 (right circle). Expression of 16 genes was commonly upregulated in the two conditions.

H) mRNA expression of 16 commonly upregulated genes in MDA-MB-231 (light grey bar, left) or MDA-MB-468 (dark grey bar, right) after CM primed MDM supernatant stimulation; dotted black line represents normalized expression level in untreated control samples (N=3, Mean±SEM).

*p<0.01, **p<0.001, ***p<0.0001, *****p<0.000001; (D, H) Student's t-test.

FIG. 16. High expression of SIGLEC1/CCL8 is associated with poor outcomes in breast cancer patients. Heatmap and disease-specific survival of SIGLEC1 and CCL8 gene expression on A) the breast cancer stroma dataset (Finak et al., 2008), B) the METABRIC cohort and C) the ER+/HER2− patients from the METABRIC cohort. All significant cut-points (p<0.05) are shown in black. Black vertical lines indicate positivity for ER and HER2 expression or grade III tumors.

FIG. 17. Schematic representation of the crosstalk between Br-TAM and cancer cells. Tumor cells up-regulate SIGLEC1, TNFα and CCL8 expression in Br-TAM. In turn cancer cells respond to CCL8 stimulation by producing CSF-1, IL1β and TNFα, which further contribute to the positive feedback loop.

FIG. 18. A box and whisker plot showing, per patient, mean distance (in μm) to T-Cell for CD163 Mac (left) and CD169 Mac (right). Median values are indicated by a white bar and are 163.26 μm and 71.38 μm for CD163 Mac and CD169 Mac, respectively. Lower and upper quartile shoulders are 96.85 μm & 163.26 μm, respectively, for CD163 Mac-to-T-Cell, and 47.75 μm & 71.38 μm, for CD169 Mac-to-T-Cell. Mean values are CD163=290.88 μm and CD169=104.58 μm (not shown). The mean distance of CD169 Mac-to-T-Cell is significantly smaller than CD163 Mac-to-T-Cell, with a P value of 0.004.

### Experimental Results

1. Materials and Methods

Patient and Control Samples

All study protocols were approved by the IRB of the Albert Einstein Medical College (Bronx, N.Y., USA), by The University of Edinburgh (Edinburgh, UK) and Duke University (Durham, N.C.) ethics committees as appropriate. Informed consent was obtained from all human subjects included in this study.

Cohort 1: Breast cancer tissue (0.1-1 grams) and endometrial cancer tissue (0.1-1 grams) was obtained from Montefiore Medical Center, NY, USA. Normal breast tissue from mammoplasty reduction surgeries (25-50 grams) was obtained from the Human Tissue Procurement Facility (HTPF), Ohio State University, USA; normal/benign endometrial tissue (1-2 grams) was obtained after surgery for conditions unrelated to cancer from Montefiore Medical Center, NY, USA.

Cohort 2: Cancer tissue (0.1-1 grams) was obtained from breast cancer patients from NHS, Edinburgh, Scotland, UK. Normal/benign breast tissue (0.5-1 grams) from patients with benign conditions was obtained from NHS, Edinburgh, Scotland, UK.

Cohort 3: Breast cancer tissue was obtained by Duke University, Durham N.C., USA. Pathologically the breast cancer patients consisted of invasive breast cancers with either node⁻ or node⁺ disease. Patients had biopsy-confirmed invasive tumors of at least 1.5 cm at diagnosis. Tumor samples were shipped on ice to Oregon Health & Science University Hospital (OHSU) for immune and genomic assays.

The exclusion criteria for all cancer patients at baseline included systemic metastatic disease, any inflammatory disorder, and active infection or immunocompromised status not related to cancer. All the patients recruited were chemotherapy and radiotherapy naive before collection.

CLINICAL DETAILS OF COHORT 1 AND 2

| Variables | Cohort1 | Cohort 1 | Cohort 2 | Cohort 2 |
|---|---|---|---|---|
| No of patients | 60 | 46 | 65 | 52 |
| Age, Years, median | 65 | 48 | 60 | 45 |
| (range) | (42-81) | (38-62) | (23-86) | (27-72) |
| Gender | | | | |
| Male | 0/60, | 0/46, | 0/65, | 0/52 |
| Female | 60/60 | 46/46 | 65/65 | 52/52 |
| Type of cancer | | | | |
| Breast | 40/60, | n/a | 65/65, | n/a |
| Endometrium | 20/60 | n/a | 0/65 | n/a |
| Breast Cancer Grade (invasive) | 1 (16/40) | n/a | 1 (18/65) | n/a |
| Breast Cancer Grade (invasive) | 2 (11/40) | | 2 (25/65) | |
| Breast Cancer Grade (invasive) | 3 (13/40) | n/a | 3 (22/65) | n/a |
| Breast Cancer ER⁺ | 32/40 | n/a | 65/65 | n/a |
| Breast Cancer PR⁺ | 28/40 | n/a | 7/65 | n/a |
| Breast Cancer HerER2+ | 8/40 | n/a | 1/65 | n/a |
| Breast Cancer ER⁻/PR⁻/HerER2⁻ | 8/40 | n/a | 0/65 | n/a |
| Endometrial Cancer Grade | 1 (4/20) | n/a | n/a | n/a |
| (invasive) | | | | |
| Endometrial Cancer Grade | 2 (5/20) | n/a | 0/65 | n/a |
| (invasive) | | | | |
| Endometrial Cancer Grade | 3 (11/20) | n/a | n/a | n/a |
| (invasive) | n/a | n/a | n/a | |
| Endometrial Cancer Type 1 | 10/20 | n/a | n/a | n/a |
| Endometrial Cancer Type 2 | 10/20 | | | |

CLINICAL DETAILS OF COHORT 3

| | | Tumor | | | Node | | | | |
|---|---|---|---|---|---|---|---|---|---|
| | | Size | | | Status | | ER | PR | Her2 |
| Age | Race | (cm) | Grade | Histology | (pos/total) | Node | Status | Status | Status |
| 60 | W | 2.4 | 1 | Lobular | 0/3 | − | 8 | 7 | 1+ |
| | | | | | | | (100) | (90) | (20) |
| 70 | W | 3.5 | 2 | Lobular | 13/15 | + | 8 | 6-7 | 2+ |
| | | | | | | | (100) | (66) | |
| 71 | W | 2.1 | 3 | Ductal | 0/2 | − | 0 | 0 | 2+ |
| 42 | AA | 15.9 | 3 | Ductal | 8/21 | + | 7-8 | 6 | 1+ |
| | | | | | | | (95) | (66) | (80) |
| 55 | AA | 3.7 | 2 | Ductal | 1/12 | + | 0 | 0 | 1+ |
| | | | | | | | | | (10) |
| 82 | Other | 3 | 2 | Ductal | 5/15 | + | 8 | 3-4 | 2+ |
| | | | | | | | (100) | (1) | (70) |
| 49 | W | 1.9 | 2 | Lobular | 0/6 | − | 8 | 8 | 1+ |
| | | | | | | | (100) | (100) | (10) |
| 43 | W | 1.5 | 2 | Ductal | 0/4 | − | 6 | 3 | 1+ |
| 68 | W | 4.2 | 3 | Metaplastic | 0/6 | − | 0 | 0 | 0 |
| 50 | W | 3.7 | 2 | Ductal | 4/27 | + | 0 | 0 | 0 |
| 34 | W | 2.5 | 2 | Solid | 0/1 | − | 7-8 | 0 | 2+ |
| | | | | | | | (90) | | (30) |
| 42 | AA | 3.5 | 2 | Ductal | 14/16 | + | 0 | 0 | 2+ |
| | | | | | | | | | (20) |
| 46 | W | 1.5 | 3 | Ductal | 0/5 | − | 7-8 | 8 | 3+ |
| | | | | | | | (89-90) | (100) | (100) |
| 76 | | 5.1 | 2 | Ductal | 0/3 | − | 0 | 0 | 2+ |
| 74 | | 0.8 | 2 | Ductal | 1/15 | + | 1+ | 0 | 2+ |
| | | | | | | | (1) | | |
| 53 | W | 3 | 3 | Ductal | 4/29 | + | 0 | 0 | 3+ |
| | | | | | | | | | (100) |
| 73 | | 4.2 | 2 | Ductal | 4/14 | + | 3+ | 2-3+ | 3+ |
| | | | | | | | (100) | (50) | |
| 66 | | 9.6 | 3 | Ductal | 4/4 | + | 0 | 0 | 0 |
| 56 | | 2.6 | 3 | Ductal | 0/7 | − | 3+ | 3+ | 0 |
| | | | | | | | (>95) | (>90) | |
| 49 | | 4.3 | 3 | Ductal | 0/3 | − | 3+ | 0 | 2+ |
| | | | | | | | (90) | | |
| 36 | | 1.9 | 1 | Ductal | 4/32 | + | 3+ | 3+ | 0 |
| | | | | | | | (100) | (100) | |
| 61 | | 4.8 | 3 | Ductal | 1/17 | + | 3+ | 3+ | 2+ |
| | | | | | | | (99) | (60) | |
| 78 | | 2.2 | 3 | Ductal | 0/1 | − | 3+ | 1-3+ | 3+ |
| | | | | | | | (30) | (40) | |
| 54 | | 7 | 2 | Lobular | 5/24 | + | 3+ | 3+ | 0 |
| | | | | | | | (30) | (30) | |
| 78 | | 2.2 | 3 | Ductal | 0/0 | NP | 3+ | 0 | 2+ |
| | | | | | | | (100) | | |
| 65 | | 9 | 2 | Ductal | 8/36 | + | 1-3+ | 1-3+ | − |
| | | | | | | | (90) | (60) | |
| 65 | | 1.1 | 2 | Lobular | 0/1 | − | 3+ | 1-3+ | − |
| | | | | | | | (100) | (60) | |
| 59 | | 7 | 2 | Lobular | 4/17 | + | 3+ | 1+ | 0 |
| | | | | | | | (100) | (10) | |
| 24 | | 2.8 | 3 | Ductal | 0/17 | − | 0 | 0 | 2+ |
| 39 | | 9.9 | 2 | Ductal | 05/13 | + | 3+ | 0 | 2+ |
| | | | | | | | (>95) | | |
| 38 | | 9.2 | 2 | Ductal | 01/15 | + | 0 | 0 | 3+ |
| 51 | | 4.2 | 3 | Ductal | 0/5 | − | 2+ | 0 | 2+ |
| | | | | | | | (20) | | |
| 71 | | 3 | 1 | Lobular | 0/3 | − | 3+ | 0 | 0 |
| | | | | | | | (>95) | | |
| 47 | | 2.1 | 1 | Ductal | 0/2 | − | 3+ | 3+ | 0 |
| | | | | | | | (90) | (70) | |
| 47 | | 5.3 | 1 | Ductal | 4/12 | + | 3+ | 3+ | 0 |
| | | | | | | | (95) | (50) | |
| 60 | | 3.7 | 2 | Ductal | 2/26 | + | 3+ | 1 | 2+ |
| | | | | | | | (>90) | (10) | |
| 33 | W | 10 | 3 | DCIS | 0/5 | − | 3 | 3 | − |
| | | | | (Comedo) | | | (10) | (<1) | |
| 70 | W | 2.2 | 2 | Ductal | 4/20 | + | 1 | 2 | 0 |
| | | | | | | | (95) | (99) | |
| 70 | W | 4.6 | 2 | Lobular | 5/16 | + | 8 | 8 | 0 |
| | | | | | | | (100) | (100) | |

| | | |
|---|---|---|
| 47 | W | Prophylactic Mastectomy |
| 37 | | Prophylactic Mastectomy |
| 41 | | Prophylactic Mastectomy |

Isolation of Human Tissue Macrophages.

Cancer tissue and normal endometrial tissue were washed with Phosphate Buffer Saline (PBS) in a petri dish and tissue was chopped into small fragments with a razorblade on ice. The sample was transferred to a 15-50 ml tube according to size and Liberase enzymes TL (14 U/mL) and DL (28 U/mL) (Roche) and DNAse (15 mg/mL) (Roche) were added in serum-free PBS. Tissue was digested at 37° C. on a rotating wheel for 1-18 hr depending on tissue weight; at the end of digestion the cell suspension was filtered using a 100 μm cell strainer and PBS 1% w/v Bovine Serum Albumin (BSA, Sigma-Aldrich) was added in order to interrupt the digestion process. Cells were centrifuged at 400 RCF for 5 min at 4° C. in a swinging bucket rotor. The pellet was re-suspended in PBS, 1% w/v BSA and cells counted and stained for FACS sorting or analysis. Macrophages were sorted using the antibodies CD45 AlexaFluor-700, CD3 PE-Cy5, CD56 PE-Cy5, CD19 PE-Cy5, CD14 FITC, CD11b PE-Cy7, CD163 APC [b 7].

Flow Cytometry Sorting and Analysis

Blocking of Fc receptors was performed by incubating samples with 10% v/v human serum (Sigma Aldrich) for 1 hr on ice. For cytofluorimetric analysis 5×10⁵ cells were stained in a final volume of 100 μL using the following antibodies at 1:100 dilutions: CD45 PE-Texas Red, CD3-, CD56-, CD19-BV711, CD11b BV605, CD14 BV510, CD16 EF450, CX3CR1 FITC, HLA-DR BV650, CCR2 PE-Cy7 (Biolegend). For macrophage sorting cells were stained and antibody concentration was scaled up based on cell number; cells were stained with the following antibodies at 1:100 dilutions: CD45-AlexaFluor 700, CD3-, CD56-, CD19-PE-Cy5, CD14 FITC, CD11 b PE-Cy7, CD16 PE-Texas Red, CD163 APC (Biolegend). Cancer cell lines were stained for the 5 CCL8 receptors with the following antibodies: CCR1 PE, CCR2 PE-Cy7, CCR3 FITC, CCR5 PE, CCR8 PE (Biolegend). Cells were incubated in the dark for 1 hr on ice; after washing with PBS 1% w/v BSA (analysis) or PBS 0.1% w/v BSA (sorting) cells were filtered and re-suspended in the appropriate buffer before analysis or sorting. Cytofluorimetric analysis was performed using a 6-laser Fortessa flow cytometer (BD); FACS sorting was performed using FACS Ariall and FACS Fusion sorters (BD). Cell sorting was performed at 4° C. in 1.5 ml RNAse and DNAse free tubes (Simport, Canada) pre-filled with 750 μl of PBS 0.1% w/v BSA; at the end of each isolation a sorting purity check was performed. A minimum of 5,000 events in the monocyte/macrophage gate was acquired for cytofluorimetric analysis. Results were analyzed with Flowjo (Treestar) or DIVA software (BD)

RNA Sequencing and Bioinformatic Analysis

Immediately after sorting all the samples were centrifuged at 450 RCF for 10 min at 4C. The cell pellet was resuspended in 350 uL of RLT lysis buffer and RNA extracted with RNAeasy Microkit (Qiagen) according to manufacturer's instructions. RNA quantity was determined by QUBIT (Invitrogen); total RNA integrity was assessed by Agilent Bioanalyzer and the RNA Integrity Number (RIN) was calculated; samples that had a RIN>7 were selected for RNA amplification and sequencing. RNA was amplified with Ovation RNAseq Amplification kit v2 (Nugen) according to manufacturer's instructions; amplified RNA was sent to Albert Einstein Genomic Facility (https://www.einstein.yu.edu/departments/genetics/resources/genomics-core.aspx) or BGI (Philadelphia; http://en.genomics.cn/navigation/show_navigation?nid=271) where library preparation, fragmentation and paired-end multiplex sequencing were performed (HIseq 2000 and 2005, IIlumina). All samples were processed and randomly assigned to lanes without knowledge of clinical identity to avoid bias and batch effects.

Sequencing Alignment and Quantification

FastQ files of 2×100 bp paired-end reads were quality checked using FastQC. Samples were filtered for low quality reads (Phred score>=20) and adapters were removed when necessary using Cutadapt. Quality controlled reads were then aligned to the human reference genome (GRCh37/hg19) using STAR aligner (version 2.3). Quantification of genes was performed using the count function of HTseq. Reads were counted at the gene level and the unstranded option was used (-s no).

Statistical Analysis for Differentially Expressed Genes

All statistical calculations were performed in R programming language (version 3.2.3). For macrophage samples, genes with count per million (CPM) reads >1 in at least N samples (N number of the fewest replicates of a condition) was retained. Gene expression levels were normalized using the Trimmed Mean of M-values (TMM) method using the calcNormFactors( ) function and log₂ transformed using the cpm( )function from the EdgeR package in R. Differential expression analysis was performed with sample quality weights using the package limma-voom package in R. Differential expression analysis was performed using the limma package. Significantly differentially expressed genes (DEGs) were selected with controlled False Positive Rate (B&H method) at 5% (FDR<=0.05). Up-regulated genes were selected at a minimum log₂fold change of 1.5 and down-regulated genes at a minimum log₂fold change of −1.5. PCA plots were drawn using the TMM/log₂ transformed (macrophages) values on expressed genes. Heatmaps were drawn on the normalized expression matrix using the pheatmap package in R. Euclidean distance and complete linkage were used for hierarchical clustering. Venn diagrams were constructed based on the overlapping differentially expressed transcripts (FDR<=0.05, Log₂FC more or less than 1.5/−1.5).

Enrichment and Pathway Analysis

Gene set enrichment analysis was performed using the gsea( ) function from phenoTest package in R. The function is used to compute the enrichment scores and simulated enrichment scores for each variable and signature. For our analysis, the logscale variable was set to false, as the log₂ transformed expression values were fed into the function and 20,000 simulations were used (B=20,000). The Database for Annotation, Visualization and Integrated Discovery (DAVID) functional annotation tool was used for gene ontology and pathway (KEGG and Reactome) analysis on the list of differentially expressed genes (FDR<=0.05, Log₂FC more or less than 1.5/−1.5). Important GO terms and pathways were selected based on an FDR<=0.05.

Publicly Available Datasets

The following publicly available datasets were used in this study:

Breast cancer:
a) Karnoub et al. (GSE8977) [6]: total of 22 samples coming from breast ductal carcinoma-in-situ (DCIS) patients (n=15) and invasive ductal (IDC) breast cancer patients (n=7) were downloaded from GEO. Samples were processed and normalized using the robust Multi-Array average expression measure (RMA) from the affy package in R. Probes representing the same gene were averaged to a single value.
b) Finak et al. (GSE9014) [4]: total of 59 samples coming from breast cancer stroma patients (n=53) and healthy controls (n=6) including updated clinical information were downloaded from GEO. Technical replicates were averaged to a single array using the averarrays( ) function from limma package in R. Data were then quantile normalized using the normalizeQuantiles( ) function. Samples were annotated and probes representing the same gene were averaged to a single value.
c) METABRIC cohort [8]: Microarray gene expression data and associated clinical information (n=1980) (Loge transformed intensity values) were downloaded from the cBioPortal for cancer genomics database (http://www.cbioportal.org/) under the study name Breast cancer. Gene expression values were quantile normalized and samples with gene expression and corresponding clinical information were selected resulting in n=1353 patients. Data were filtered for missing values and samples with molecular subtype NC were removed. The filtering resulted in n=1350 patients that were used for further analysis. For survival analysis, events were censored based on disease-related deaths (Died of disease=1; Living or Died of other causes=0).
d) Cancer cell Encyclopedia (CCLE) data: Gene expression RPKM normalized reads from breast cancer cell lines (n=57) were downloaded from the CCLE website (https://portals.broadinstitute.org/ccle).

Colorectal cancer:

a)GSE14333 [9] array of colorectal cancers in surgically resected specimens in 290 colorectal cancer patients;

b)GSE17536 [10] array of 55 colorectal cancer patients from Vanderbilt Medical Center (VMC) and 177 patients from the Moffitt Cancer Center.

Lung cancer:

GSE31210 [11] Expression profiles in of 226 lung adenocarcinomas (127 with EGFR mutation, 20 with KRAS mutation, 11 with EML4-ALK fusion and 68 triple negative cases).

Quantitative PCR

Cells were lysed and RNA extracted with RNAeasy Microkit (Qiagen) according to manufacturer's instructions. Typically, 0.1 ug of total RNA was reverse transcribed using Super Script Vilo kit (Invitrogen) and the cDNA generated was used for semi quantitative PCR on a 7900 Real Time cycler (Applied Biosystem) as per manufacturer's instructions. Target gene expression was normalized to the expression of the housekeeping gene GAPDH. Relative gene expression was calculated using the standard 2-ΔΔCT method. Primers were designed using Primer Bank. The primers used are shown in Table 3.

TABLE 3
qPCR primer list

| Target | | Sequence (5′→3′) | SEQ ID No. |
|---|---|---|---|
| GAPDH | FWD | GGAGCGAGATCCCTCCAAAAT | 4 |
| | REV | GGCTGTTGTCATACTTCTCATGG | 5 |
| CX3CR1 | FWD | ACTTTGAGTACGATGATTTGGCT | 6 |
| | REV | GGTAAATGTCGGTGACACTCTT | 7 |
| SIGLEC1 | FWD | CCTCGGGGAGGAACATCCTT | 8 |
| | REV | AGGCGTACCCCATCCTTGA | 9 |
| CCR2 | FWD | TACGGTGCTCCCTGTCATAAA | 10 |
| | REV | TAAGATGAGGACGACCAGCAT | 11 |
| CCL8 | FWD | TGGAGAGCTACACAAGAATCACC | 12 |
| | REV | TGGTCCAGATGCTTCATGGAA | 13 |

Survival Analysis

For the SIGLEC1/CCL8 signature the summed normalised gene expression values were dichotomized based on the optimal cutoff calculated by iteratively calculating every possible expression cutoff (n-1) and selecting the value with the lowest p-value. For the METABRIC cohort, disease-specific survival (DSS) was used as an endpoint. For the breast cancer stroma dataset, recurrence-free survival (RFS) was used as an endpoint and censored at date of last follow-up. Survival curves were estimated using the Kaplan Meier method (survival and survminer R packages). For SIGLEC1 and CCL8 single gene survival analysis, clinical risk factors such as ER status (positive or negative), PR status (positive or negative), HER2 status (positive or negative), histological grade (I, II or III), age (greater or less than 55) and tumor size (greater or less than 50 mm) were used in the univariate and multivariate models. Candidate prognostic factors for RFS and DSS with a p-value (Wald test) lower than 0.05 in univariate analysis were used in the multivariate analysis. Multivariate analysis was performed by fitting a Cox proportional hazard regression model. The Cox regression model was used to calculate the Hazard ratio (HR) and 95% confidence internal (CI). A p-value less than 0.05 based on a Wald test was considered significant.

Immunofluorescence and Quantitation

All tissues were fixed in 4% w/v paraformaldehyde, dehydrated and embedded in paraffin blocks; 5 μm sections were cut onto positively charged glass slides and stained with the following antibodies: CD163 (Leica Biosystems NCL-LCD163, Clone 10D6) dilution 1:1000, CD169 (Novus Biologicals, NPB2-30903, polyclonal) dilution 1:100. High throughput immunofluorescence was performed by the SURF Facility at the University of Edinburgh (http://surf.ed.ac.uk/facilities/immunodetection-and-histological-imaging/) after primary antibody optimization. Immunofluorescently stained tissues were batch-scanned on a Zeiss AxioScan.Z1 (Carl Zeiss, Oberkochen, Germany) with specific scan profiles for each stain group and using a 40× Plan-Apochromat 0.95NA coverslip corrected air objective. Slide scanned images were imported into a Definiens Tissue Studio workspace (Definiens AG, Munich, Germany) and pre-processed for nuclear detection and cell simulation using built-in nuclear detection and cell growth algorithms. The pre-processed workspace was then imported into Definiens Developer XD (Definiens AG, Munich, Germany) for further processing, quality control, machine learning, and k-Nearest Neighbour classification and output compiled in Mathematica 10.3 (Wolfram Inc., Champaign, Ill., United States) and tabulated in a spreadsheet. Incomplete or low-quality nuclei and cells were discarded using a combination of DAPI pixel intensities and standard deviation. For CD163, examples of 300 cells each were given for positive and negative cases in a single large tissue sub-region of one cancer tissue previously identified to show the most variation of intensity. These class samples were used to optimize a feature space consisting of 49 subjectively selected morphological, textural, statistical, and intensity-based metrics. Feature space optimization indicated 19 features as being most important for separation of both populations using a Euclidean distance matrix. A classifier algorithm was used to compile these 19 metrics for each given class sample in each population and then used to classify all remaining cells in that tissue. A selection of at least 10 incorrectly classed cells were then manually corrected and added to the relevant class sample populations before recompiling the 19-dimensional feature space and reclassifying the whole tissue. This iterative learning process was repeated at least 10 times with a final sample size of 400-500 cells for each class. The classifier was stored as .xml and used to batch classify the entire data set of tissues from all patients.

Multiplex Immunohistochemistry

Staining: Formalin-fixed paraffin embedded (FFPE) tissue samples (5 μm) were used for iterative multiplex immunohistochemistry as previously described [12]. Briefly, following standard antigen retrieval and blocking, primary antibodies (listed in Table 4 below) were applied to tissue sections and incubated overnight at 4° C. Primary antibodies were detected using a species-specific F(ab′) fragment-specific secondary antibody-labeled polymer-based peroxidase system (Histofine, Nichirei Biosciences Inc, Japan) in conjunction with 3-amino-9-ethylcarbazole (AEC). Images were acquired using Aperio ImageScope AT (Leica Biosystems) and slides were subject to iterative cycles of staining.

Image processing and analysis: All image processing and analysis was performed as previously described [12] on three regions/slide, which encompassed the total tissue area. Image cytometry was performed using FCS Express 5 Image Cytometry (De Novo Software) and cell populations were determined using multiparameter cytometric image analysis (see gating schema). Cell populations were normalized to total cell number (Cells/Total Cells) and populations were quantified. Unsupervised hierarchical clustering was performed using R package pheatmap_1.0.8. Correlation was used as a distance measure and average was used as clustering method.

TABLE 4
Antibodies used for multiplex IHC

| Target | Company/Product# | Clone | Dilution |
|---|---|---|---|
| CD45 | eBiosciences: 14-0459-82 | H130 | 1:100 |
| CD3 | ThermoFisher Scientific: MA1-90582 | SP7 | 1:150 |
| CD8 | ThermoFisher Scientific: MA5-13473 | 08/144B | 1:100 |
| CSF1R | abcam: ab183316 | SP211 | 1:150 |
| CD169 | Millipore: MABT328 | 5F1.1 | 1:200 |
| CD163 | ThermoFisher: MA5-11458 | 10D6 | 1:100 |
| CD56 | Santa Cruz Biotech | 123C3 | 1:100 |
| CCR2 | Novus: MAB150-SP | 48607 | 1:400 |

Monocyte Derived Macrophages (MDM) Isolation and Stimulation

Peripheral blood was collected from healthy donors in EDTA coated blood tubes and diluted 1:2 using serum free PBS. 40 mL of the diluted blood was then stratified on top of 10 mL of Ficoll; samples were centrifuged at 400 RCF (no brake, no acceleration) for 30 min at room temperature (RT) in a swinging bucket rotor. The peripheral mononuclear cell (PBMC) fraction (ring) was collected with a pipette and cells washed with PBS [13]. PBMC were counted and seeded in a 12-well plate (NUNC-BD) at the concentration of 8×10⁶cells/ml for 2 hr at 37° C. 5% v/v CO₂ in serum free medium (Dulbecco's Modified Eagle Medium, DMEM). Non-adherent cells were removed and wells washed twice with PBS and 2 ml of DMEM 10% v/v Fetal Bovine Serum (Lonza), 5% v/v Human AB serum (Lonza) and 1% v/v penicillin/streptomycin were added to each well; 50% of the medium (1.0 ml) was replaced with fresh medium every 3 days. After 7 days of differentiation monocyte-derived macrophages (MDM) were treated for 24 hr with MDA-MB-231 and MDA-MB-468 cancer cell derived supernatant (CM) as reported in the below sections [14]. After 24 hr all the supernatant was removed and used for quantitative real-time (qPCR) metastasis breast cancer array (see below), cells were washed twice with PBS and lysed with Trizol Reagent (Thermo Fisher) for RNA extraction; RNA was extracted using Trizol manufacturer's protocol. RNA was converted to cDNA using Invitrogen Superscript Vilo cDNA synthesis kit and qPCR was performed using the protocol described above in the text.

IPSC Derived Macrophages

The SFCi55 iPSC line was generated in house and was confirmed to be pluripotent and have a normal karyotype [15]. The cells were maintained in StemPro medium prepared by supplementing DMEM/F12+ Glutamax (Invitrogen) with StemPro hESC supplement (Invitrogen), 1.8% BSA (Invitrogen), 0.1 mM β-mercaptoethanol (Invitrogen) and 20 ng/ml human basic FGF (Invitrogen). Differentiation of iPSCs to macrophages was carried out as previously described (Lopez-Yrigoyen et al., 2018). When iPSC colonies covered approximately 80% of the culture surface, (Day 0), spent medium was removed and replaced with 1.0 ml StemPro supplemented with cytokine Mix 1 (50 ng/ml BM P4, 50 ng/ml VEGF, and 20 ng/ml SCF). Colonies were cut using the EZPassage™ tool, and gently dislodged with a Pasteur pipette. They were divided equally into two wells of an Ultra-Low Attachment 6-well plate (Corning), and 2 ml of fresh StemPro media with cytokine Mix 1. Cells were cultured in suspension until day 4 with a cytokine top up on Day 2, to make embryoid bodies (EBs). On Day 4, EBs were lifted and transferred to gelatin-coated tissue-culture grade 6-well plates in X-VIVOTM 15 media (Lonza) supplemented with cytokine Mix 2 (100 ng/ml CSF1, 25 ng/ml IL3, 2.0 mM Glutamax, 1% Penicillin/Streptomycin, 0.055 M β-mercaptoethanol). 10 to 15 EBs were plated in each well. EBs were maintained in this medium for the remainder of the protocol, with spent medium being replaced with fresh medium every 3-4 days. After about 2-3 weeks, the EBs produced macrophage progenitors in the culture supernatant that were harvested and transferred to 10 cm² bacteriological dishes in X-VIVOTM 15 medium supplemented with cytokine Mix 3 (100 ng/ml CSF1, 2.0 mM Glutamax, 1% v/v Penicillin/Streptomycin) and allowed to mature for 7 days into iPSC-derived macrophages (iPSC-DM). Macrophage progenitors were harvested every 4 days for approximately 2 months.

THP-1 Monocyte Differentiation and Cytokine Stimulation

Human THP-1 monocytes were maintained in culture medium (10% v/v Fetal Bovine Serum [FBS] Roswell Park Memorial Institute [RPMI]1640 Medium) and incubated at 37° C. in a 5% v/v CO₂ atmosphere. For monocyte-macrophage differentiation, cells were seeded in at a density of 2.5×10⁵ cells/ml on 12-well plates, or 5×10⁵ cells/ml in 6-well plates and macrophage differentiation was initiated by exposing the cells to 5ng/ml phorbol-12-myristate-13-acetate (PMA) (Sigma-Aldrich, 16561-29-8) in 10% v/v FBS culture medium at 37° C. in a 5% v/v CO₂ atmosphere for 24 h. Subsequently, THP-1 derived macrophages were polarized using different combinations of IL-4, IL-10, IL-13 and TGF-β (InvivoGen, USA) or using different pro-inflammatory cytokines including TNFα, IFNγ IL-β, IL-6 and IL-12 (InvivoGen, USA). The cytokines doses were 20 ng/ml and LPS was used at 25 ng/ml.

Cancer Cell Culture, Conditioned Medium Production and Cytokine Stimulation

MDA-MB-468 and THP1 cell lines were cultured in (RPM11640 with 10% v/v serum (GIBCO, Life Technologies); MDA-MB-231 cells were cultured in DMEM with 10% v/v serum (GIBCO, Life Technologies). All cells were originally obtained from ATCC (Manassas, Va., USA) and subsequently maintained in our laboratory. All cell lines were frequently tested for mycoplasma contamination using a commercially available Mycoplasma detection kit (Myco alert kit, Lonza, USA), and all tested negative. To obtain MDA-MB-231 and -468 CMs cells were resuspended in culture medium, seeded at a density of 1×10⁵ cells/ml in 2.5 ml culture medium on 6-well plates and cultured overnight at 37° C. in a 5% v/v CO₂ atmosphere. Subsequently, for CM exposure on PMA-THP-1 monocytes, culture medium was replaced with 10% v/v FBS RPM1640 medium, for CM exposure on human monocyte-derived macrophages (MDMs), culture medium was replaced with 10% v/v FBS DMEM supplemented with 5% v/v human serum and for CM exposure on human iPSDM culture medium was replaced with 10% v/v FBS DMEM. After medium change, cells were cultured for an additional 24 h with fresh medium and thereafter, cell free supernatants were harvested and directly used for the experiment.

CCL8 MRNA, SIGLEC1 and CD163 Protein Detection

Mixed multiplex staining (RNAscope, Tyramide dual immunofluorescence) for CCL8 mRNA, CD169 (SIGLEC1) and CD163 protein detection was performed on a Leica RX research-staining robot (Newcastle, UK). RNAscope (ACD Bio Newark, Calif.) was performed using manufacturers recommendations using ACD LS2.5 Brown kit (322100) as follows. FFPE fixed breast cancer needle biopsies were dewaxed in xylene and rehydrated through graded ethanol, following a brief rinse in water sections were washed in tris buffered saline containing 0.01% v/v tween 20 (TBST). Slides were placed onto a Leica RX robot and stained using the manufacturer's recommended LS2.5 Brown RNAscope protocol. mRNA integrity was assessed using PPIB (cat 313908) using the following standard tissue pretreatments: ACD ER2 for 10 min with ACD Protease 5 min or ACD ER2 at 95° C. for 15 min with ACD Protease 15 min or ACD ER2 at 95° C. for 20 min with ACD Protease 25 min. Mild conditions (ACD ER2 95° C. 10 min) with ACD protease (5 min) were assessed as providing optimal mRNA detection whilst maintaining both protein antigenicity and tissue section morphology of these relatively delicate sections. Following tissue pretreatment the standard recommended protocol was followed, briefly AMP1 30 min, AMP2 15 min, AMP3 30 min, AMP4 15 min, AMP5 30 min and AMP6 15 min followed by visualisation in DAB for 20 min using standard recommended washes. Modification of standard protocol for CCL8 (466498) to obtain a fluorescent endpoint involved omitting DAB substrate and replacing with Tyramide Cy5 at 1:100 dilution (Perkin Elmer, NEL745001KT, Seer Green, UK).

Following CCL8 mRNA detection using RNAscope with Cy5, sections were sequentially stained for CD169 protein with Tyramide Cy3 detection and CD163 with Tyramide FITC detection using heat elution between detections for specificity. Using a Leica RX robot slides were subject to further Heat Induced Epitope Retrieval (HIER) using ER1 retrieval buffer for 10 min at 99C, followed by blocking in 3% Hydrogen Peroxide and 20% Normal Goat Serum. CD169 (Novus Biologicals, NBP2-30903, Cambridge, UK) was added to sections at 1:100 dilution for 60 min followed by secondary antibody Goat anti Rabbit Peroxidase fab at 1:500 dilution (Abcam, ab7171, Cambridge, UK) before visualisation with Tyramide Cy3 at 1:50 dilution. (Perkin Elmer, NEL744E001KT). Stripping of antibodies from the tissue sections was performed for 10 min at 99C followed by blocking in 3% v/v Hydrogen Peroxide and 20% v/v Normal Goat Serum. CD163 (Leica Biosystems NCL-LCD163, Clone 10D6) was added to sections at 1:1000 dilution for 60 min followed by secondary antibody Goat anti Rabbit Peroxidase fab at 1:500 dilution (Abcam, ab7171) before visualisation with Tyramide FITC (Perkin Elmer, NEL741001KT) at 1:50 dilution and counterstaining with DAPI at 1:1000 dilution. All washes between incubations were for 2×5 min in TBST [16].

ELISAS

Human CCL8, TNFα, and IL1β protein levels were quantified by Duoset ELISA kits (R&D systems, USA) following manufacturer's instructions. Human CSF1 protein levels were quantified by quantikine ELISA kit (R&D systems, USA). All cell culture supernatants were used undiluted. CCL2 levels were assessed in plasma from 15 healthy donors and 42 breast cancer patients using Legendplex bead-based immunoassays (Biolegend) according to manufacturer's protocol. Data were collected using the C4 Accuri (BD). ELISA for human CX3CL1 was done using a human CX3CL1 Quantikine ELISA kit (R&D Systems) as per manufacturer's instructions.

Cytokine Array

Human Cytokine ELISA Plate Array (Signosis, EA-4002), consisting of one pre-coated plate able to detect 32 cytokines simultaneously for 3 independent human samples was used to quantify cytokines in supernatants from MDMs before or after cancer CM stimulation. Detection of cytokines produced from MDMs before or after CM stimulation was performed based on the manufacturers instructions) (Sigma-Aldrich, 16561-29). 8.0 μl of MDM supernatants from each group was added into each well of the plate and incubated at room temperature for 2 h. After washing, 100 μl of diluted biotin-labeled antibody mixtures were added into each well for another one hour incubation. After washing again, each well was incubated with detection antibody mix and then HRP, and the plate was read on a plate reader at 450 nm.

IPSDM-Cancer Cell Conditioned Medium Production

Human iPSDM culture medium was replaced with 10% v/v FBS DMEM 24 h before CM incubation. iPSDM were incubated with MDA-MB-231 and -468 CMs (prepared as described above) for 24 h and then medium was changed; after medium change, cells were cultured for an additional 24 h with fresh medium and thereafter, cell free supernatants were harvested and directly used for the experiment.

TNFA Neutralization in IPSDM-Cancer Cell Conditioned Medium

iPSDM-Cancer cell conditioned medium was incubated for 24 h with 1.0 μg/ml of mouse anti human TNFα neutralizing antibody (R&D systems, USA, MAB210-SP, Clone 1825) or 1.0 pg/ml of mouse IgGi isotype control (R&D systems, USA, MAB002). Efficacy of anti-TNFα antibody neutralization was tested by TNFα ELISA before use.

PCR Arrays

PCR-based microarrays for evaluating the expression of genes mediating the inflammatory response were performed using the human inflammatory cytokines and receptors RT2 Profiler TM PCR array (Qiagen, PAHS-011ZE-4); PCR-based microarrays for evaluating the expression of genes in breast cancer cell lines were performed using the Breast cancer PCR array RT2 Profiler TM PCR array (Qiagen, PAHS-131Z-4) and the Tumor Metastasis PCR array RT2 Profiler TM PCR array (Qiagen, PAHS-028Z). The arrays were configured in a 384-well plate consisted of a panel of 92 genes and 4 endogenous genes. Reverse transcription was performed using the RTC First Strand Kit (Qiagen, 330401) and qPCR was performed using RTC SYBR Green/ROX PCR Master mix (Qiagen, 330521), and the raw data were analyzed by the GeneGlobe Data Analysis Center (www.qiagen.com) according to the manufacturer's instructions.

Cell Proliferation Assay

Cell proliferation was determined using the Cell Counting Kit (CCK)-8 assay (Sigma-Aldrich, 96992) according to the manufacturer's instructions. A total of 5,000 cells were seeded into each well in the 96-well plates and allowed to attach overnight. Cells were then treated with 0.1 ng/ml, 1.0 ng/ml or 10 ng/ml CCL8 (R&D Systems, 281-CP-010/CF). After the treatment (6 to 72 hours), a CCL8 solution was added to each well and then cells were incubated at 37° C. for 2 h. Cell proliferation was measured using the microplate reader and the proliferation of cells was defined as OD450-OD620.

In Vitro Cell Migration Assay

The “scratch” assay to assess cell migration was performed following previously published protocols [17]. Cells were grown in DMEM with 10% v/v FBS in 12-well plates until they reached confluence; after 24 h of starvation (DMEM 0% FBS), a scratch was performed using a p200 Eppendorf tip. Recombinant CCL8 and CCL2 were used at 1 ng/ml concentrations in all the experiments. Cells were filmed for 24 h in a 37° C. thermostatic chamber using an Axiovert Scope. 2-3 independent sections/well were filmed and 4 independent experiments per condition were performed. Data analysis was performed with Image J (NIH).

Chemotaxis Assay

THP1 chemotaxis was performed using Essen Biosciences reagents. THP1 cells were cultivated in RPMI medium with 10% FBS and seeded at 4000 cells/well in 96 well chemotaxis plates in the presence or absence of 20 ng/ml recombinant human CCL2 or CCL8 (R&D systems). Migration was recorded every hour for 72 h using the IncuCyte system (Essen Bioscience) and number of cells migrated was calculated using IncuCyte quantification software.

Determination of Interactions Between Cells in Clinical ER+ Breast Cancer Samples Derived from Immunofluorescent Stained Sections

Patient biopsy samples were sectioned, stained, and imaged for component cell analysis. Cell objects were detected in image analysis software. Each cell object was then classified as positive or negative for three immunofluorescence markers, CD163, CD169 (Siglec1), and CD8, using individual machine learning models for each marker. Cells with CD163 positivity only were classified as “CD163 Mac”. Cells with both CD163 and CD169 were classified as “CD169 Mac”. Cells with CD8 positivity were classified as “T-Cells”.

The distance to the nearest T-Cell for every CD163 Mac and CD169 Mac was then calculated and the population means were taken for each pairing in each patient biopsy sample. E.g. All CD163Mac-to-T-Cell distances were averaged for Patient1. Likewise, CD169-Mac-to-TCell distances were averaged for Patient1. This process was repeated for Patient2-Patient26.

The average distances of CD163 Mac-to-T-Cell and CD169 Mac-to-T-Cell were then compared statistically using a T-Test.

Statistics

Statistical significance was calculated by Student's t-test when comparing two groups or by one-way or two-way ANOVA when comparing three or more groups. A p-value<0.05 was considered as statistically significant.

2. TAMS from Breast and Endometrial Cancers Exhibit A Distinct Transcriptional Profile from Resident Macrophages

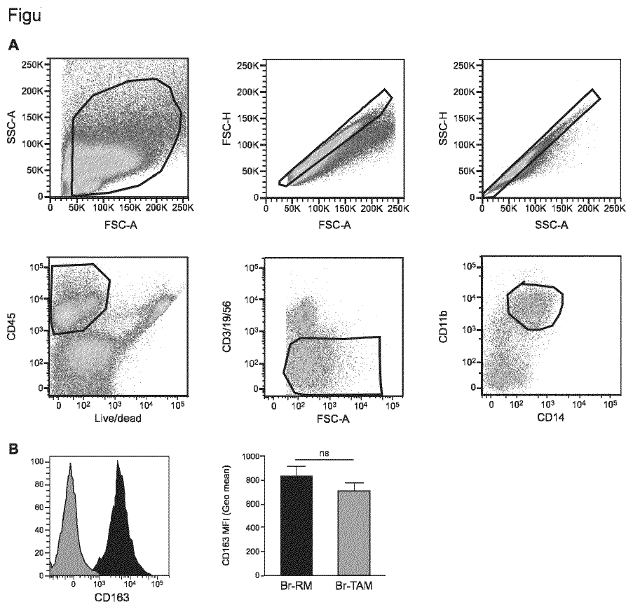

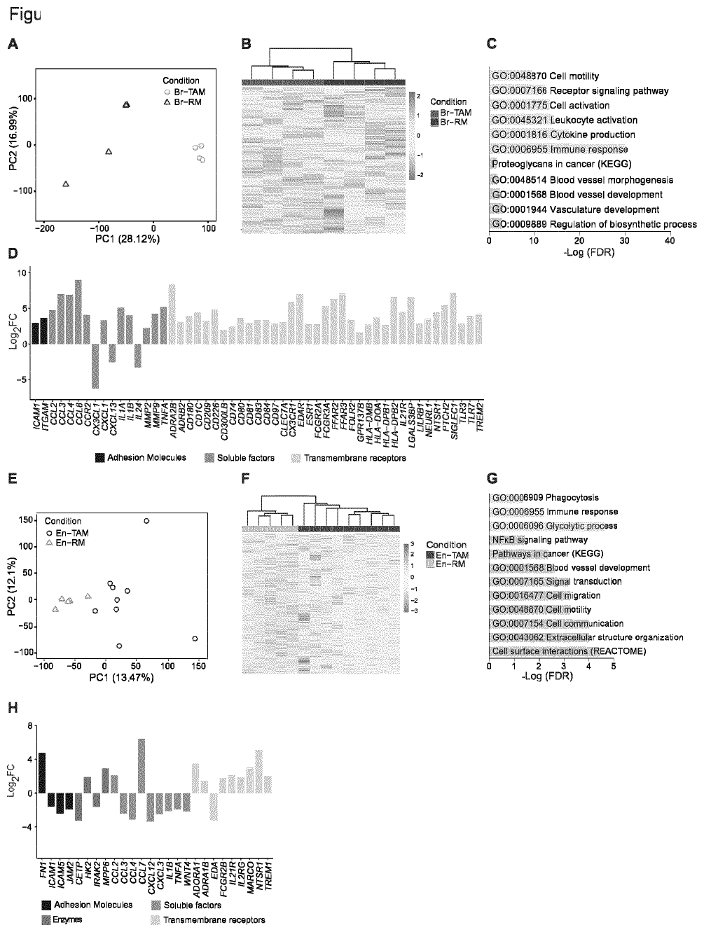

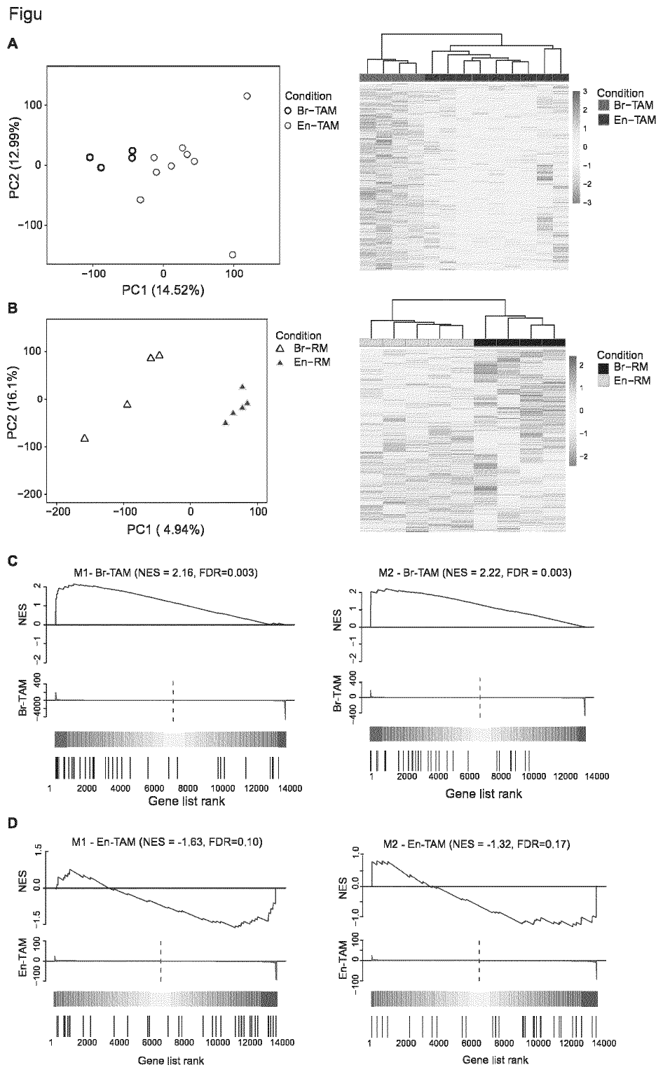

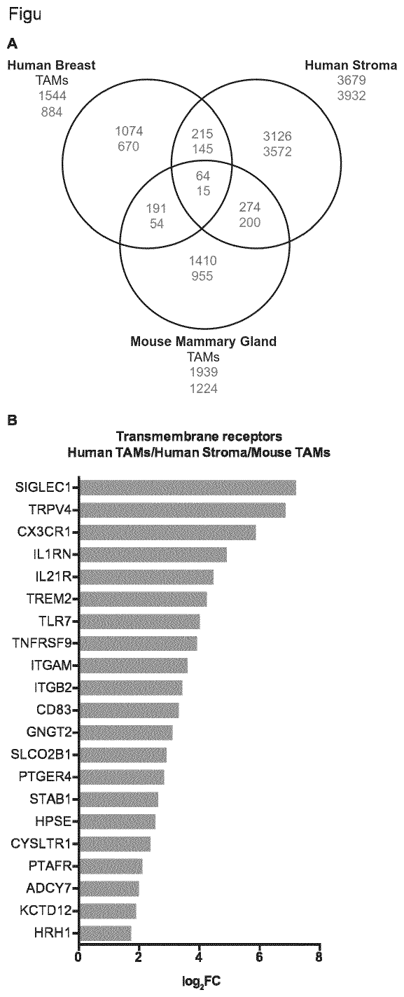

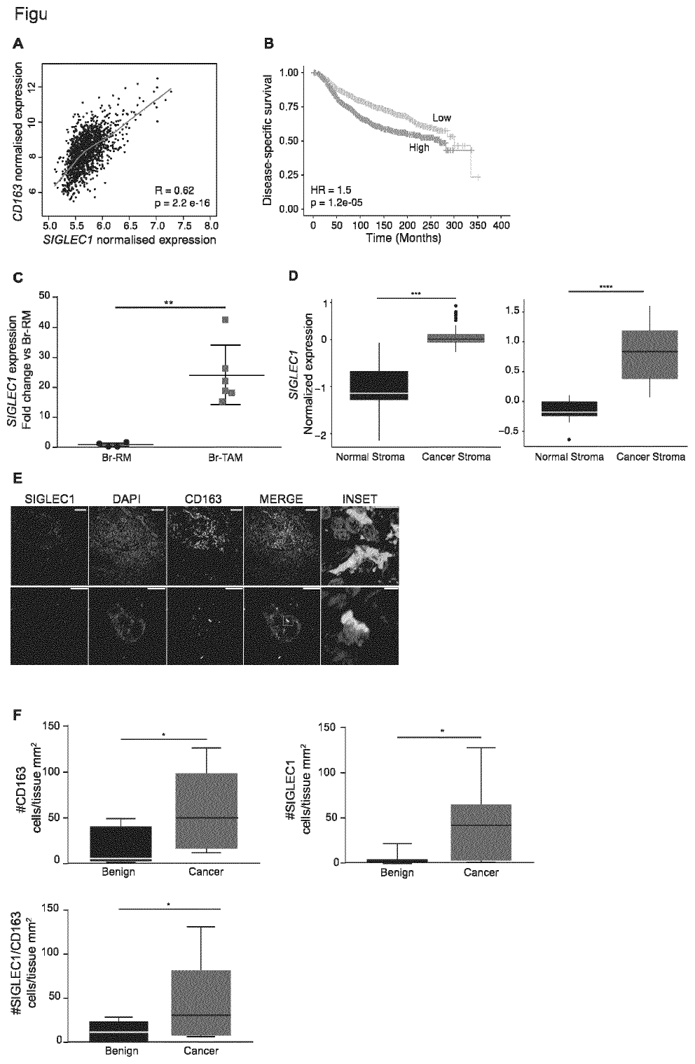

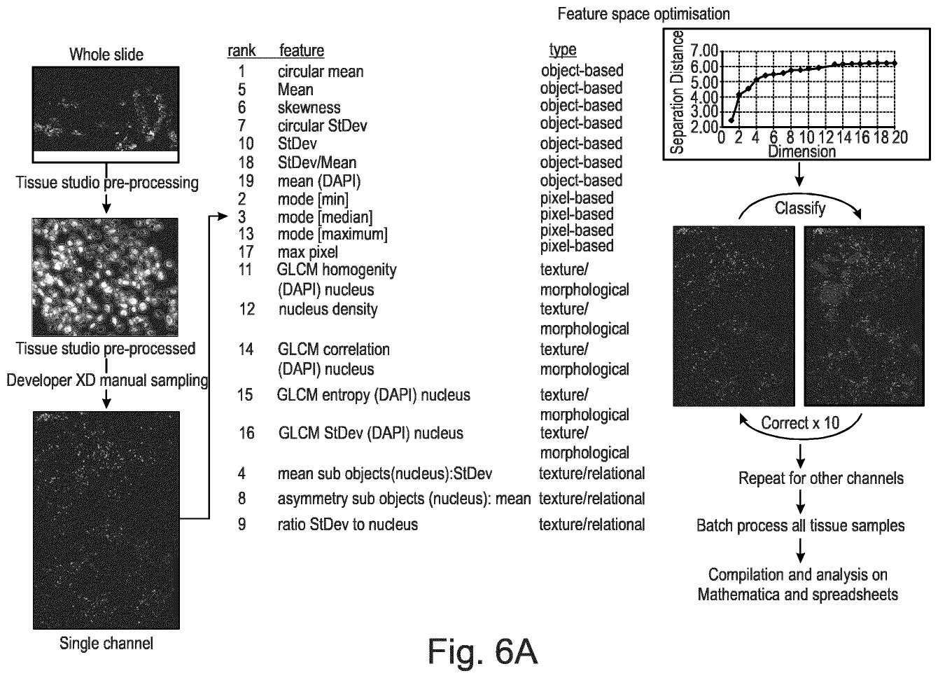

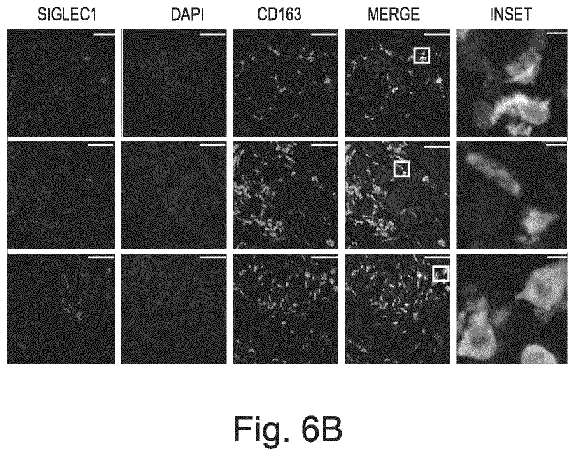

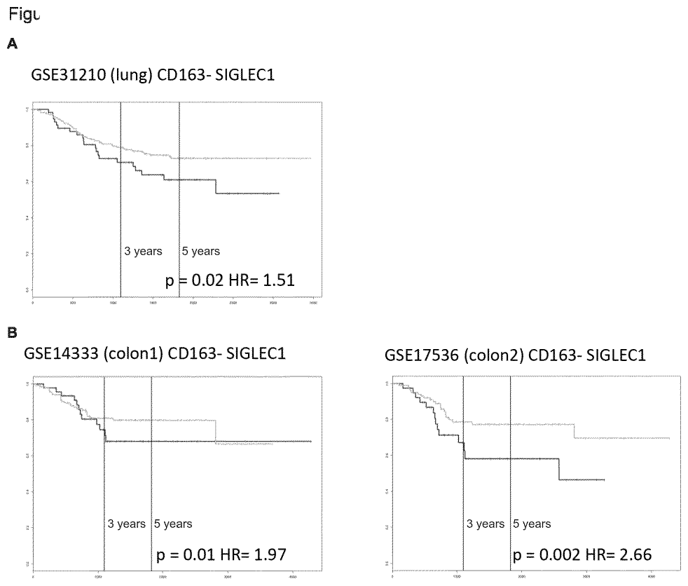

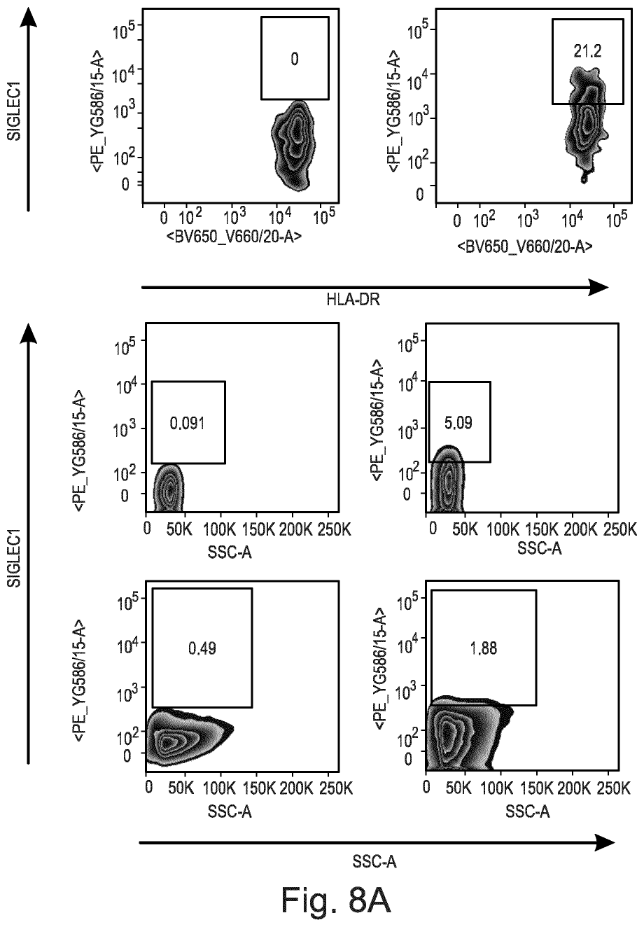

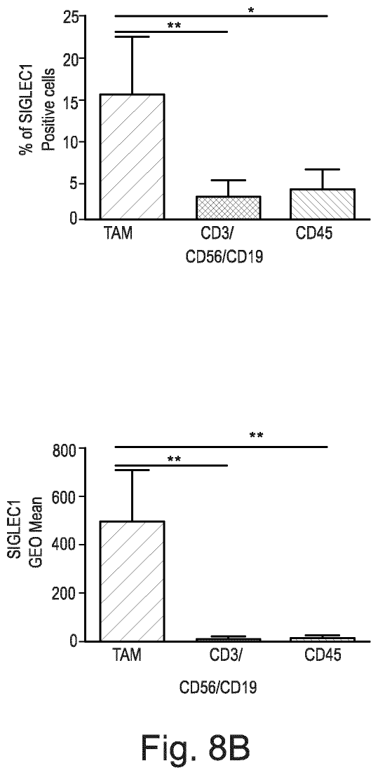

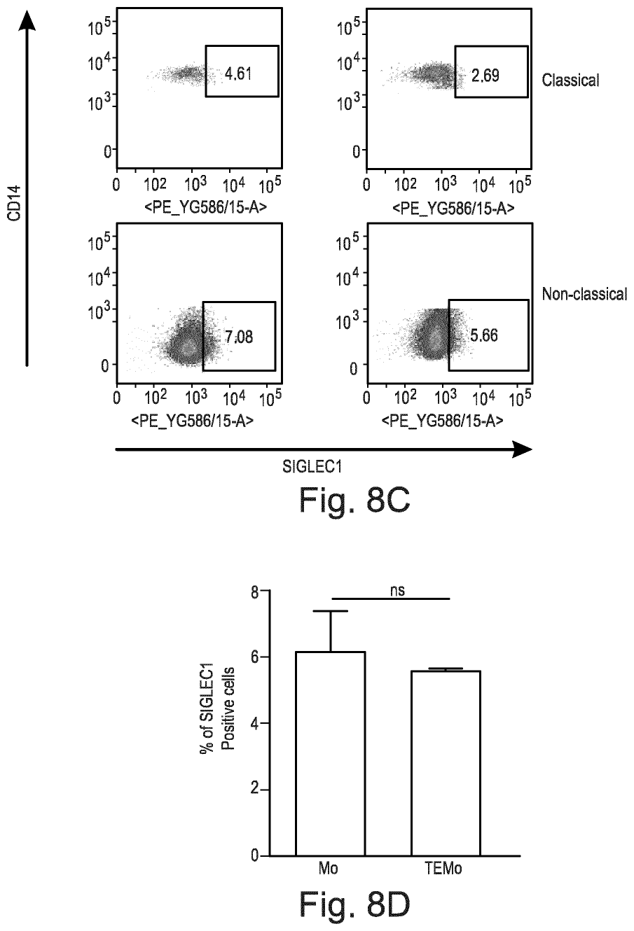

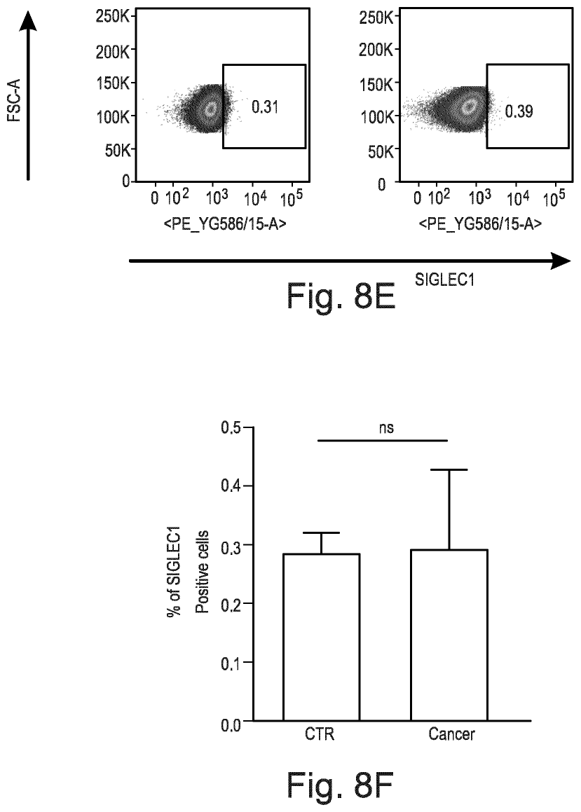

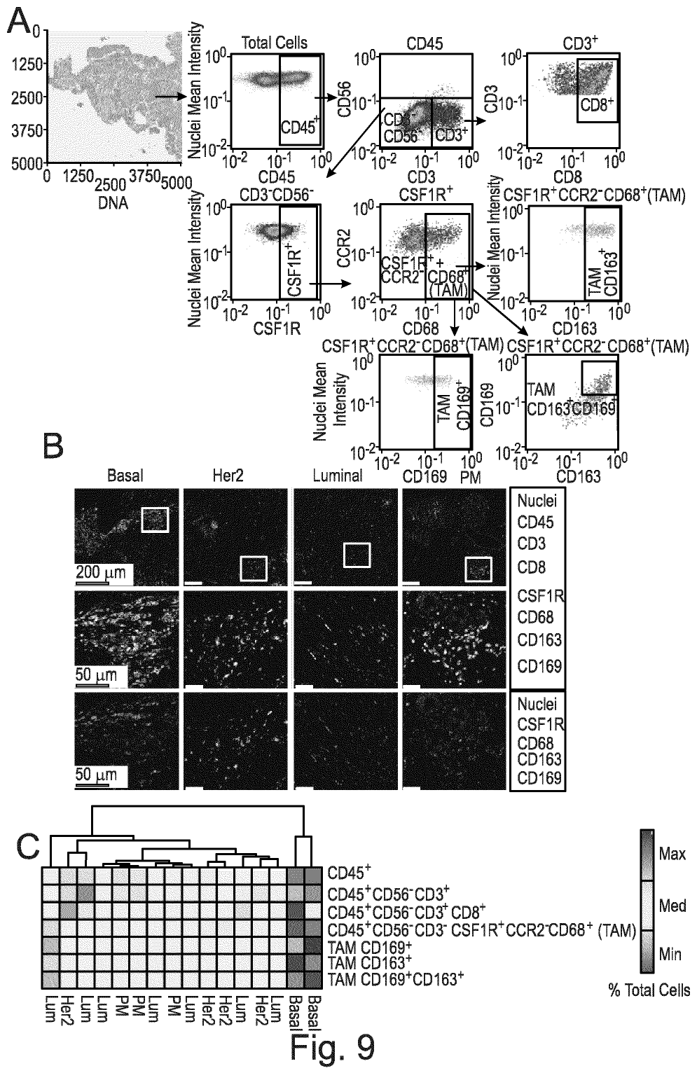

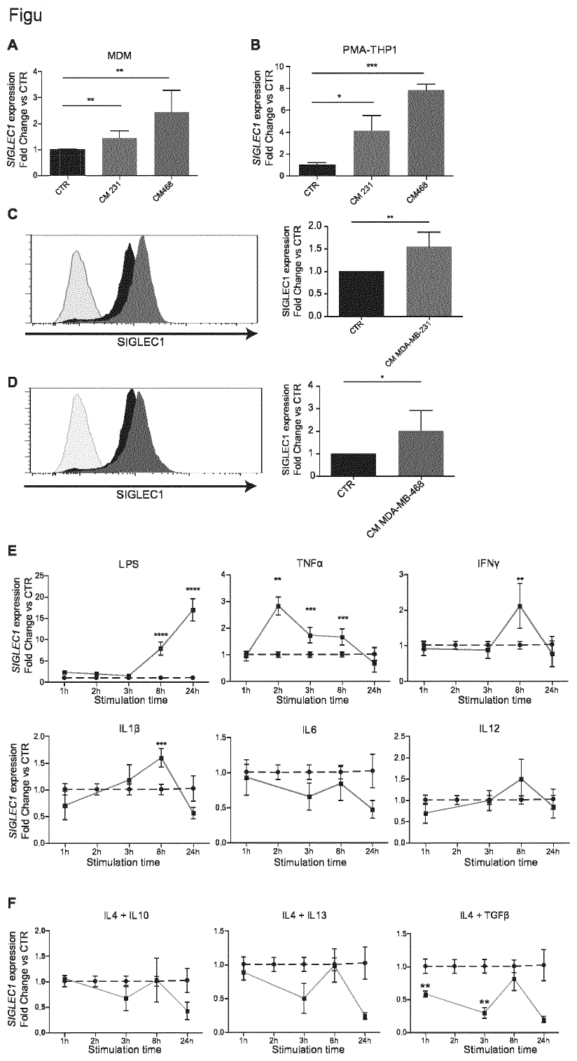

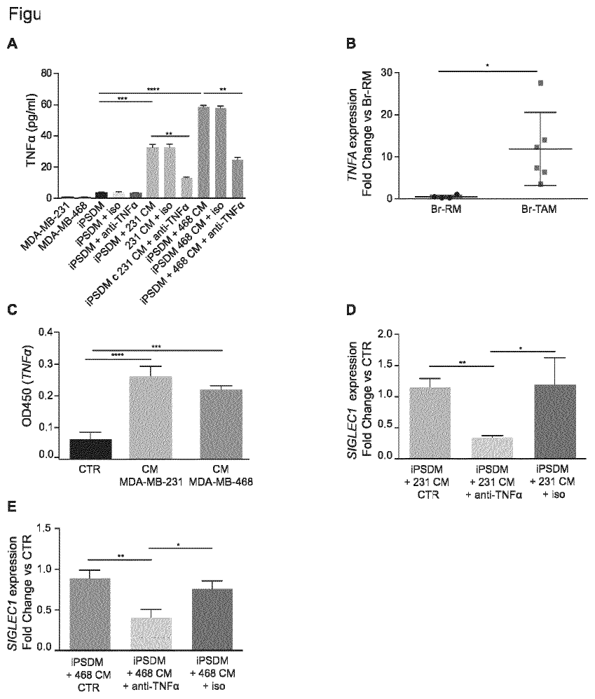

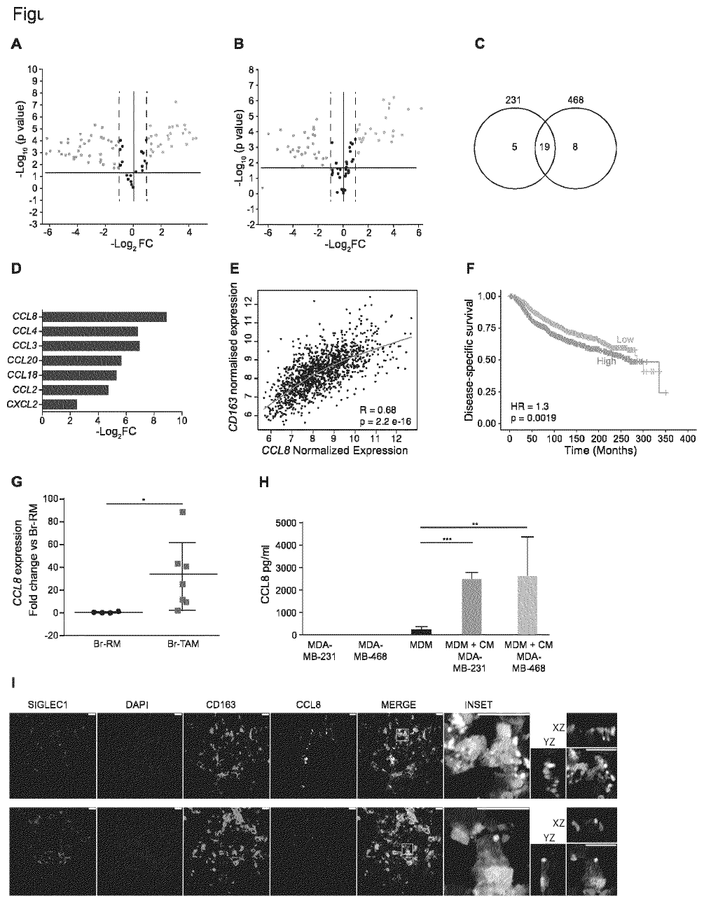

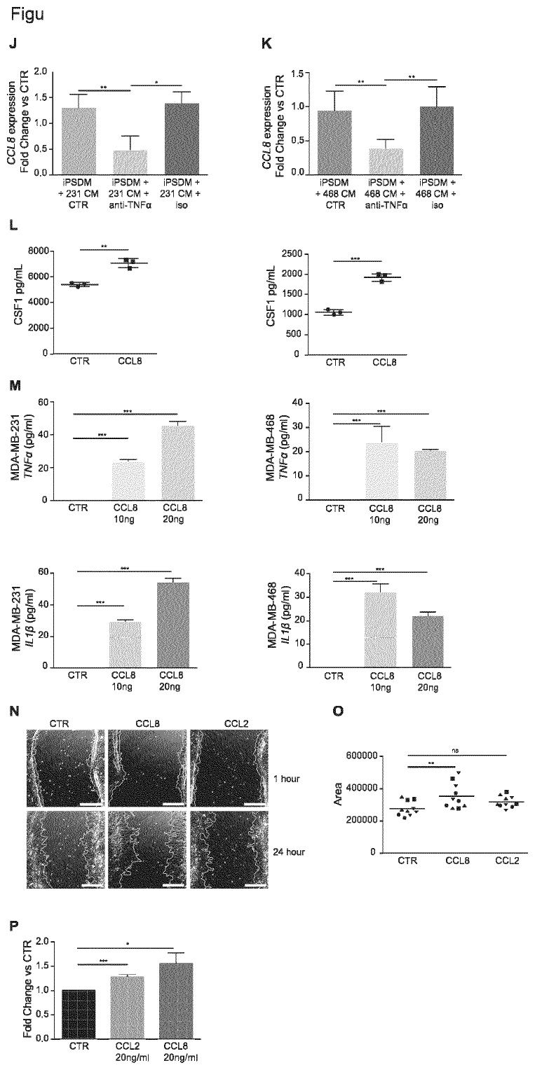

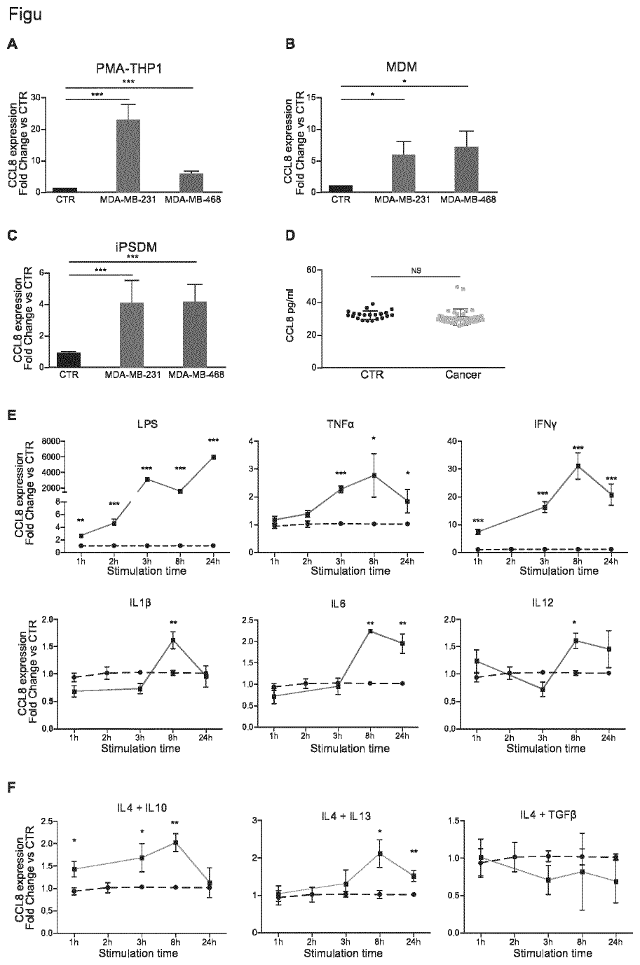

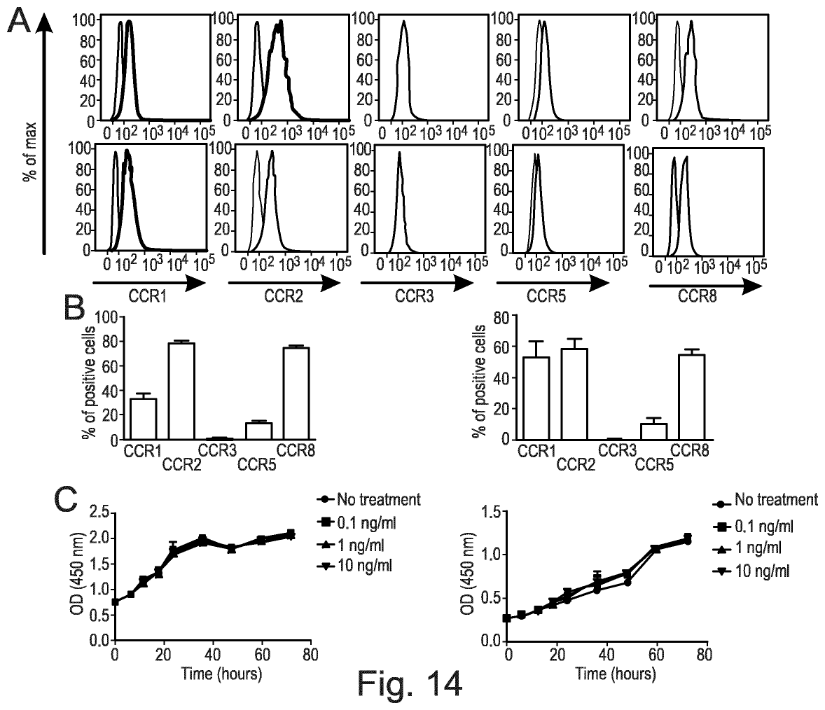

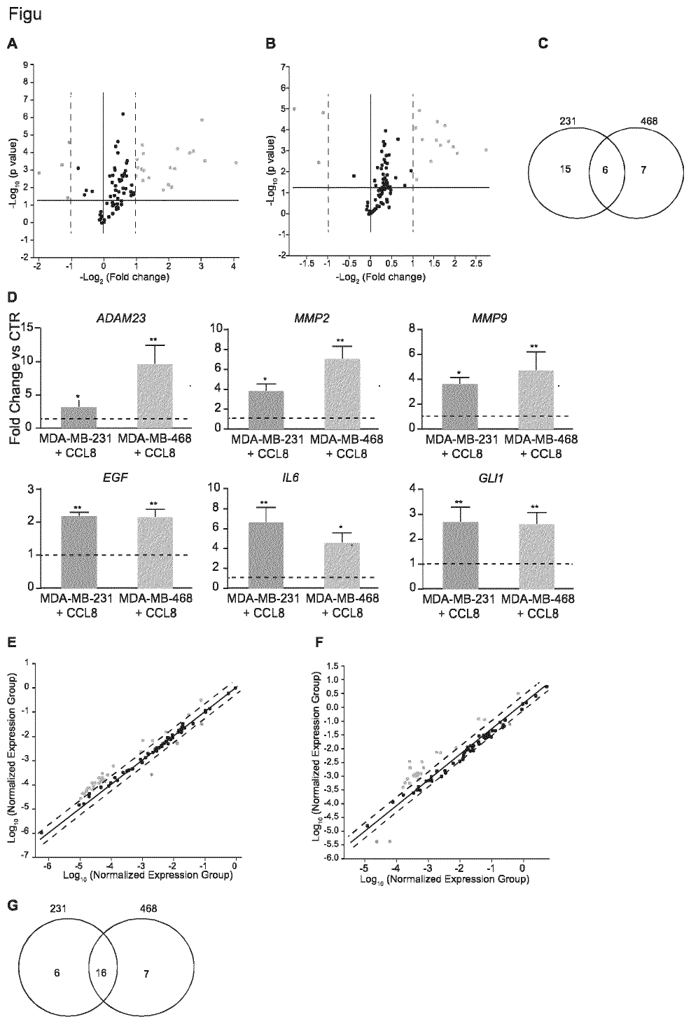

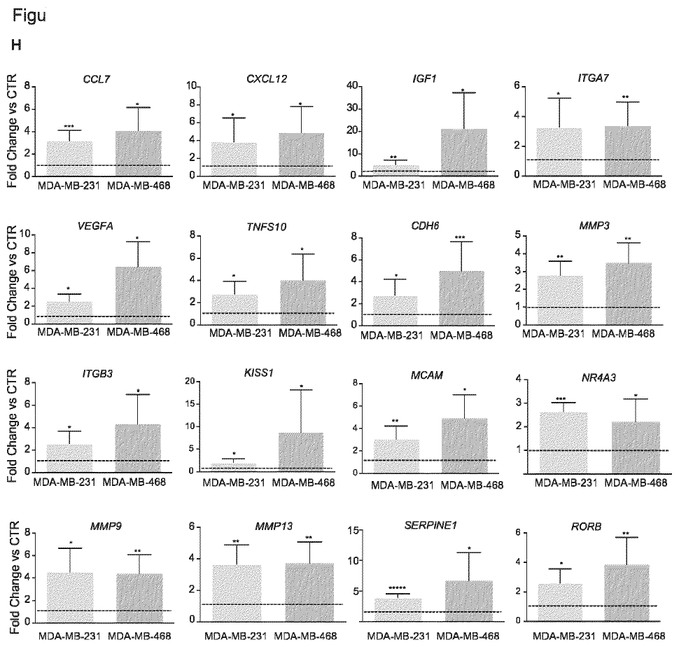

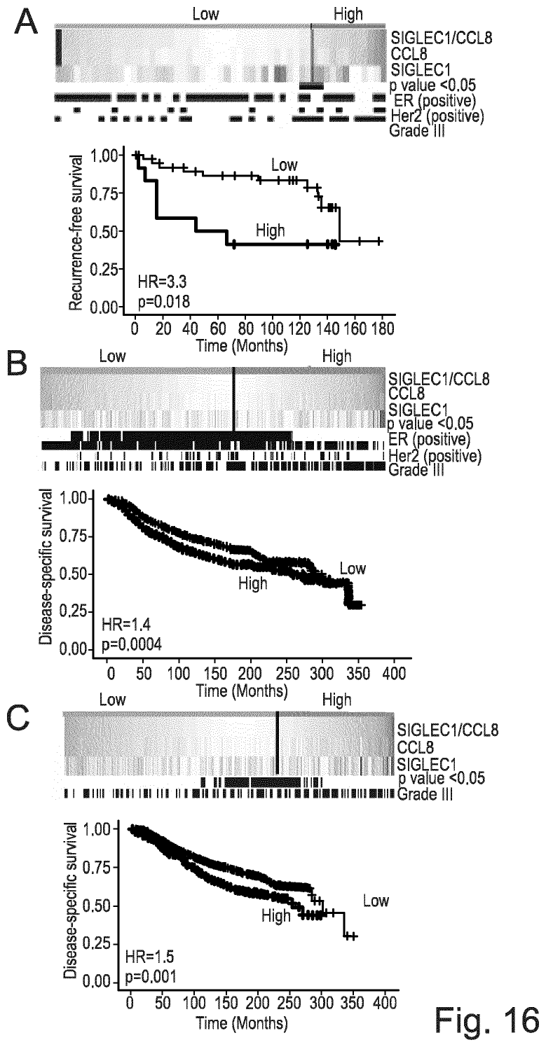

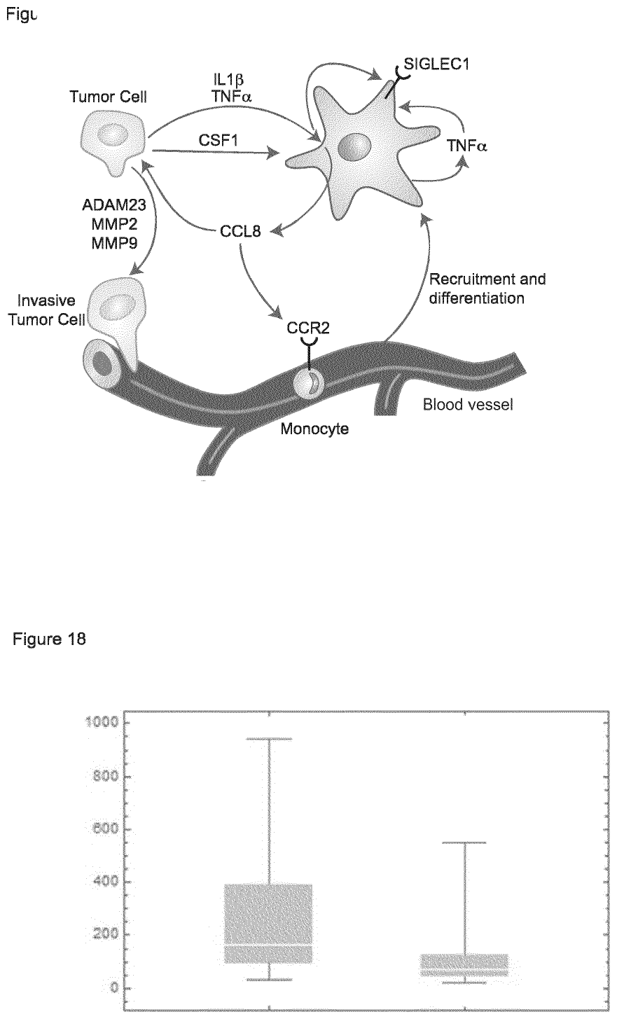

There is significant evidence showing pro-tumoral profiles of TAMs in mouse models of cancer; however, a detailed characterization of their transcriptomes and phenotypes in human cancers is still lacking. Thus, we analyzed TAM transcriptomes by RNA-seq from breast and endometrial cancer in comparison to resident macrophages from homeostatic tissue after FACS sorting (FIG. 1A). PCA and hierarchical clustering revealed distinct clusters of breast tissue resident macrophages (Br-RM) and breast cancer TAMs (Br-TAM) (FIG. 2A, 2B). Limma DEA revealed 1873 DEGs in Br-TAM compared with Br-RM (1301 up and 572 down; FDR<=0.05). Gene ontology (GO) analysis reported several enriched GO terms such as cell motility and activation, vasculature development and immune response (FIG. 2C). Br-TAM showed increased transcript abundance of genes encoding transmembrane receptors associated with immune cell activation and antigen presentation such as MHC class II molecules, Fc receptors, T cell co-stimulatory molecules (CD80 and CD83), TLRs and Ig receptor superfamilies, and TREMs (FIG. 2D). Although in mice CD163 is often referred to as a TAM marker, we did not observe a significant difference in CD163 expression between Br-RM and Br-TAM (FIG. 1B).

PCA and hierarchical clustering revealed distinct clusters of endometrial tissue resident macrophages (En-RM) and endometrial cancer TAMs (En-TAM) (FIG. 2E, 2F). Limma DEA between En-RM and En-TAM identified 831 DEGs (115 up and 716 down; FDR<=0.05). GO analysis reported several enriched GO terms such as phagocytosis, immune response, cell communication, migration and blood vessel development (FIG. 2G). Additionally, a number of genes encoding transmembrane receptors, soluble factors, transcription factors and enzymes were differentially expressed; the scavenger receptor MARCO, TREM1, FCG2RB and IL21RG were up-regulated in En-TAM as compared to En-RM (FIG. 2H).

To better understand TAMs in different cancer types, we compared the gene expression profiles of Br-TAM and En-TAM. PCA and hierarchical clustering revealed two distinct groups (FIG. 3A) with very few DEGs commonly up- and down-regulated (18 genes up and 35 down), indicating that breast and endometrial cancers activate cancer tissue-specific transcriptional profiles in TAMs. Resident macrophages from endometrial and breast tissue also exhibited a distinct transcriptional profile confirming the diversity of tissue macrophage phenotypes in homeostatic states (FIG. 3B).

Macrophages exhibit distinct phenotypes in response to environmental stimuli and have been classified into two alternative polarization states, referred to as ‘M1’ and ‘M2’ with the latter being immune suppressive and pro-tumoral [3]. To determine whether these polarization states exist within human En- and Br-TAM, we performed gene set enrichment analysis (GSEA) using the M1/M2 signature as proposed by Martinez et al. Neither Br- nor En-TAM showed a preferential enrichment for M2-associated genes supporting the idea that TAM phenotypes are much more complex and cannot be simply categorized into binary states (FIG. 3C, 3D). Similarly, canonical markers for M2 that have been identified in mice, such as arginase, were minimally, and not differentially expressed in either Br- or En-TAM.

3. Datasets Meta Analysis Identified Potential New TAM Associated Markers.

TAM density is associated with markers of poor prognosis in many human cancers including breast and endometrium¹⁶. As breast cancer data sets are more abundant and available for mammary cancers in mice, we analyzed TAM transcriptional datasets to determine whether markers predictive of prognosis could be identified. Finak et al determined a stromal signature for breast [4] and Ojalvo et al determined the transcriptomes of TAMs that promote tumor cell invasion in the Polyoma middle T mouse model of breast cancer [18]. The availability of these datasets and the new one produced herein allowed us to perform a screening of transmembrane receptors upregulated on human and murine TAMs. Selecting significantly DEGs (Log₂FC 1.0/−1.0, FDR<0.05) commonly regulated in all three datasets (FIG. 4A), and in order to identify breast TAM-specific surface biomarkers, we focused on transmembrane receptor genes commonly expressed among all three datasets (FIG. 4B).

4. SIGLEC1 (CD169) Expression is Upregulated in Breast TAMS.

We selected SIGLEC1 (CD169), as it was one of the top up-regulated genes in Br-TAM (Log₂FC=7.2, FDR=0.0017) compared to Br-RM and it was also correlated with expression of the pan-macrophage marker CD163 (R=0.62, p=2.2e-16) (FIG. 5A). In the METABRIC cohort, univariate analysis showed that SIGLEC1 high expression was significantly associated with shorter disease-specific survival (HR=1.5, p=1.2e-0.5, FIG. 5B). Consistent with this, in Cox multivariate analysis after adjusting for clinical parameters such as ER, PR, HER2, grade and tumor size, SIGLEC1 high expression was independently significantly associated with shorter disease-specific survival (HR=1.42, p=1.85e-0.4,). Internal validation by qPCR confirmed the significant up-regulation of SIGLEC1 mRNA observed in the RNA-seq analysis (FIG. 5C). Furthermore, SIGLEC1 showed significantly higher expression in breast tumor stroma compared to normal breast stroma in the two datasets available (FIG. 5D).

We performed immunofluorescent (IF) staining using anti-SIGLEC1 and anti-CD163 antibodies on tissue biopsies from patients with invasive breast cancer and benign lesions (FIG. 5E). Using machine-learning image analysis (FIG. 6A) for unbiased quantification, we were able to segment and classify CD163 and SIGLEC1 single- and double-positive populations and determine their numbers within whole and sub-regions of the tissue sections. Cancer tissues had higher numbers of macrophages per mm² tissue area, and a higher percentage of SIGLEC1-positive cells compared to benign tissue (FIG. 5F); results that were further confirmed by confocal microscopy of the stained sections (FIG. 6B). These results indicate that SIGLEC1 is a human breast TAM-associated marker. SIGLEC1 and CD163 are also associated with poor prognosis in additional cancers such as colon and lung (FIG. 7A, 7B).

We used multicolor flow cytometric analysis to determine SIGLEC1 expression at the protein level in an independent cohort of breast cancer patients and found that SIGLEC1 was expressed on Br-TAM, but not on other immune cells or CD45⁻ non-immune cells, indicating specificity to macrophages/TAMs (FIG. 8A, 8B). In the circulation, classical and non-classical monocytes (FIG. 8C, 8D), but not granulocytes (FIG. 8E, 8F), exhibited low expression of SIGLEC1 with no difference between cancer and non-cancer patients.

5. SIGLEC1 Positive Macrophages Accumulate in Basal and HER2 Breast Cancers

To investigate expression of SIGLEC1 in different breast cancer subtypes we performed multiplex immunohistochemistry [12] on breast cancer tissues that had been independently acquired from cohort 3. Using image cytometry we identified 3 distinct Br-TAM subtypes (CSFR1+CCR2−CD68+CD163+SIGLEC1−, CSFR1+CCR2−CD68+CD163+SIGLEC1+and CSFR1+CCR2−CD68+CD163−SIGLEC1+, FIG. 9A) confirming results reported in (FIG. 5F). Quantification of these three Br-TAM populations revealed enrichment in Basal tumors compared to HER2 and luminal subtypes, while the three subsets were almost absent in tissues from prophylactic mastectomies (FIG. 9B, 9C). This is consistent with the increased expression of the TAM signature in aggressive breast tumors at the mRNA level.

Next, we investigated the regulation of SIGLEC1 expression in human macrophages using human monocyte-derived macrophages (MDM), induced pluripotent stem cell (iPSC) derived macrophages (iPSDM), and THP1 cells differentiated into macrophages using PMA (PMA-THP1). All three were exposed to conditioned medium (CM) from MDA-MB-231 and MDA-MB-468 cell line derived from triple negative breast cancers (Neve et al., 2006). CM from both cell lines increased expression of SIGLEC1 mRNA in MDM and PMA-THP1 (FIG. 10A, 10B). Additionally, CM enhanced SIGLEC1 protein expression on the cell surface of iPSDM (FIG. 10C, 10D). These results indicated that cancer cells actively enhance the expression of SIGLEC1 on human macrophages.

In order to further investigate the stimulus generated by cancer cells we stimulated PMA-THP1 macrophages with a panel of pro-inflammatory and anti-inflammatory cytokines and measured SIGLEC1 expression by qPCR. The inflammatory mediator positive control, Lipopolysaccharides (LPS) and the pro-inflammatory cytokine Tumor Necrosis Factor a (TNFα) were the main modulators of SIGLEC1 expression, while Interleukin 1β (IL1β) and Interferon γ (IFNγ ) produced a modest effect (FIG. 10E). Conversely, anti-inflammatory cytokines did not affect SIGLEC1 expression in a significant way, except for a down-regulation after combined exposure with IL4 and TGFβ (FIG. 10F).

In order to investigate if cancer cells produce TNFα, we performed ELISA on MDA-MB-231 and MDA-MB-468 CM, but did not detect significant levels of this cytokine (FIG. 11A). In contrast, qPCR analysis indicated a significant up-regulation of TNFα at the mRNA level in Br-TAM compared to Br-RM (FIG. 11B). Consistent with this elevated expression in Br-TAM, MDM and iPSDM incubated with either MDA-MB-231 or MDA-MB-468 CM produced significantly higher levels of TNFα compared to untreated controls at the protein level (FIG. 11C). We next neutralized TNFα in MDA-MB-231 and MDA-MB-468 CM-treated iPSDM (FIG. 11A) and exposed new iPSDM to the neutralized CM. TNFα neutralization resulted in a significant reduction of SIGLEC1 expression compared to isotype control treated CM (FIG. 11D, 11E). These results indicate that Br-TAM respond to cancer signals by up-regulating the expression of SIGLEC1 and by producing TNFα that further supports SIGLEC1 expression in macrophages.

6. CCL8 is a Breast TAM Marker

In order to identify additional mediators of the cross-talk between human cancer cells and macrophages we performed a qPCR array for inflammatory proteins on PMA-THP1 cells incubated with either MDA-MB-231 or MDA-MB-468 CM (FIG. 12A, 12B). PMA-THP1 exposed to the different CMs commonly up-regulated the expression of 19 pro-inflammatory genes (FIG. 12C). By comparing this list with genes up-regulated in Br-TAM we identified seven factors commonly up-regulated (FIG. 12D). Among these 7 factors CCL8 (Monocyte Chemotactic protein-2 or MCP-2), was the most significantly up-regulated factor in the Br-TAM dataset. Interestingly, CCL8 has been reported to play a role in the tumor microenvironment by supporting mouse mammary cancer cells dissemination (Farmaki et al., 2016). In our data, CCL8 was correlated with CD163 expression (R=0.68, p=2.2e-16) (FIG. 12E). In the METABRIC cohort, univariate analysis showed that CCL8 high expression was significantly associated with shorter disease-specific survival (HR=1.3, p=0.0019, FIG. 12F). However, in Cox multivariate analysis, after adjusting for clinical parameters such as ER, PR, HER2, grade and tumor size, high CCL8 expression was not independently significantly associated with shorter disease-specific survival (HR=1.16, p=0.13,). Internal validation by qPCR on samples used for RNA-seq showed significant up-regulation of the CCL8 transcript in Br-TAM (FIG. 12G). We next validated by incubating PMA-THP1 macrophages, MDM and iPSDM with cancer CM and assessed CCL8 mRNA and protein levels (FIG. 13 A-C and FIG. 12H). In addition, CCL8 fluorescence in situ hybridization (FISH) analysis of breast cancer tissue sections revealed that CCL8 mRNA is found in Br-TAM but not in cancer cells (FIG. 12I). There were no differences in CCL8 serum levels between normal and cancer patients indicating local production (FIG. 13D). CCL8 production in human macrophages was induced by both pro-inflammatory and anti-inflammatory stimulation (FIG. 13E, 13F) consistent with reports using cultured mouse macrophages [19].

Similarly to the observations with SIGLEC1, TNFα modulated the expression of CCL8. We neutralized TNFα in MDA-MB-231 and MDA-MB-468 CM-treated iPSDM with neutralizing antibodies and exposed new iPSDM to the neutralized CM. TNFα neutralization resulted in a significant reduction of CCL8 expression compared to isotype control treated CM, confirming a role for TNFα in CCL8 regulation in macrophages exposed to cancer cell CM (FIG. 12J, 12K). CCL8 treatment of both cancer cell lines significantly up-regulated the expression of CSF1, at both the mRNA and protein level (Log₂FC>1, p<0.05, FIG. 12L), as well as TNFα and IL1β (FIG. 12M). These results indicate that CCL8 is a marker of human breast TAMs and that cancer cells are able to modulate its expression in these cells by a TNFα-dependent mechanism. In turn, cancer cells respond to CCL8 by producing the macrophage survival and proliferation factor (CSF-1) and pro-inflammatory mediators, which further propagate the auto-stimulatory loop.

7. CCL8 Enhances Breast Cancer Cell Motility And Monocyte Recruitment

We investigated the effect of CCL8 on cancer cells. Cancer cell lines were analyzed for expression of the five reported CCL8 receptors (FIG. 14A, 14B). Of these CCR1, 2, 5 and 8 were detected on the cell surface of both MDA-MB-231 and MDA-MB-468 cells. CCL8 receptors, mainly CCR1 and CCR2, have also been shown to be expressed upon tumor cells in human breast cancers. Stimulation with recombinant CCL8 (rCCL8) did not affect cell proliferation of either breast cancer cell line (FIG. 14C). We stimulated MDA-MB-231 and MDA-MB-468 with rCCL8 and performed a qPCR array for genes associated with breast cancer progression. Using stringent criteria for changes in gene expression (Log₂FC>2, p<0.05) (FIG. 15A, 15B), six genes were identified that were commonly upregulated in both cell lines following stimulation with rCCL8 (FIG. 15C). The product of these genes have been predicted to be involved in cancer cell invasion (MMP2, MMP9, ADAM23) [20] and progression (IL6, EGF, GLI1) [19] (FIG. 15D). Similar genes were identified by a metastasis qPCR array after exposure of MDA-MB-231 and MDA-MB-468 with CM from cancer-cell primed MDM (FIG. 15E-15G, 15H). Consistent with the upregulated expression of genes involved in invasion, rCCL8 treatment enhanced motility of MDA-MB-231 cells (FIG. 12N, 12O) to a greater extent than previously reported for CCL2. Finally, as TEMo express CCR2 as the only CCL8 receptor differentially expressed, we assessed the ability of CCL8 to recruit monocytes using an in vitro chemotaxis assay with THP1 monocytic cells in the presence of CCL2 and CCL8 as chemo-attractants. Both CCL2 and CCL8 attract these monocytic cells compared to controls (FIG. 12P).

8. SIGLEC1/CCL8 Gene Signature is an Independent Prognostic Factor in ER+ Breast Cancer

To assess whether a 2-gene SIGLEC1/CCL8 signature had clinical relevance in breast cancer, Cox proportional hazard regression analysis was performed on a breast cancer stroma dataset [4] representing 53 patients suffering 17 recurrence events reported over a median follow-up time of 8.7 years. Gene expression values of SIGLEC1/CCL8 were dichotomized into high and low expression groups according to all possible cutoffs [21]. Univariate analysis revealed that SIGLEC1/CCL8 high expression was associated with shorter recurrence-free survival (RFS) (HR=3.3, p=0.018) (FIG. 16A). To further validate the clinical relevance of the SIGLEC1/CCL8 gene signature, we utilized the METABRIC cohort (N=1350) with 456 breast-cancer specific events over a median follow-up time of 9.69 years. In univariate analysis high expression of SIGLEC1/CCL8 was significantly associated with shorter disease-specific survival (HR=1.4, p=4e-04) (FIG. 16B), along with HER2 status (HR=2.1, p=2.3e-10), grade (HR=1.8, p=1.9e-09) and tumor size (HR=1.8, p=0.002). Conversely, ER (HR=0.6, p=7.8e-07) and PR (HR=0.64, p=3e-06) status were significantly associated with better disease-specific survival. In Cox multivariate analysis, SIGLEC1/CCL8 high expression was associated with shorter disease-specific survival but didn't reach significance (HR=1.2, p=0.06).

In a subset of ER positive patients from the METABRIC cohort (N=960), univariate analysis revealed that SIGLEC1/CCL8 high expression was significantly associated with shorter disease-specific survival (HR=1.5, p=0.001), along with grade (HR=1.7, p=9e-06) and age (HR=1.5, p=0.002) (FIG. 16C). Cox multivariate analysis demonstrated that SIGLEC1/CCL8 high expression was independently significantly associated with shorter disease-specific survival (HR=1.35, p=0.014) along with, grade (HR=1.54, p=3.4e-04) and age (HR=1.44, p=0.008).

9. CD163 CD169/SIGLEC1 Positive Macrophages are Significantly Closer to CD8 T Cells than CD163 Positive Macrophages

We investigated interactions between cells in clinical ER+ breast cancer samples derived from immunofluorescent stained sections. The average distance of CD169-CD163 double positive tumour associated macrophages to T-Cells (104.58 μm) is smaller than that of CD163-only positive tumour associated macrophages (290.88 μm) (FIG. 18). This indicates that, on average, CD169+ve TAMs are more closely interacting with CD8 T-Cells than their CD169−ve counterparts. These data strongly support the assertion that CD16(positive macrophages are regulating T cells and that targeting them is likely to alleviate the T cell suppression.

10. Discussion

In mouse models of cancer, cells of the mononuclear phagocytic system play profound roles in shaping the tumor microenvironment to one that promotes malignancy. Despite the large number of studies in mice and clinical correlative data suggestive of similar roles in human cancers for TAMs, there is little data describing their phenotypes. In the present work, we have shown that in breast and endometrial cancers TAMs respond to the presence of malignancy by altering their transcriptomes and therefore showing distinct profiles compared to healthy women. Detailed analysis and mechanistic studies showed paracrine signalling interactions between tumor cells and TAMs that involve SIGLEC1 and CCL8, two TAM markers that in multivariate analysis were independent predictors of disease-specific survival in ER positive patients.

In mouse models of cancer, monocytes are recruited to primary or metastatic tumors where they differentiate to TAMs that promote tumor progression and metastasis [22]. However, little is known about TAMs in human cancers. We profiled TAMs from samples belonging to a cohort of patients and defined their transcriptional landscape through bulk RNA-seq. Surprisingly, TAM transcriptomes from endometrial and breast cancers are distinct from each other, from their respective resident macrophages and their progenitor monocytes. These data suggest the existence of cancer specific niches at the tissue level that influence the TAM transcriptional profile according to tumor location and subtype. Multiplex IHC identified at least TAM sub-populations (CSFR1+CCR2−CD68+CD163+SIGLEC1−, CSFR1+CCR2−CD68+CD163+SIGLEC1+and CSFR1+CCR2−CD68+CD163−SIGLEC1+) in breast cancer patient samples and showed that there is considerable heterogeneity in TAM populations within the tumor. Although macrophages were traditionally classified into M1 and M2 polarization states, more recent studies describe a spectrum of activation states [23] and an association of both states with tumor progression [24]. Indeed, our analysis failed to reveal a unique polarization state in human TAMs. A recent study that analyzed the transcriptomes of CD45+ cells from healthy tissue and breast tumors at the single cell level (Azizi et al., 2018), confirmed the heterogeneity of myeloid cells. However, unlike this study the moderate depth of scRNA-seq studies doesn't allow for identification of novel markers.

We focused on transmembrane receptors included in the TAM signature, of which, SIGLEC1, a sialic binding receptor mainly expressed by macrophages, was the most highly differentially expressed in our Br-TAM dataset as well as in publicly available breast cancer stroma datasets. In homeostatic conditions, SIGLEC1 positive macrophages are mainly localized in the bone marrow, liver, spleen, colon and lymph node [2] and they are thought to be involved in erythropoiesis and modulation of adaptive immune responses. Consistent with our findings, SIGLEC1 positive macrophages have been identified in colorectal [25] and hepatocellular carcinoma [26]. Interestingly, while infiltration of SIGLEC1 positive macrophages in colorectal cancer was associated with tumor progression, in hepatocellular carcinoma they predicted favourable patient outcomes.

In order to elucidate the crosstalk between human TAMs and breast cancer cells, we focused on soluble factors produced by TAMs in response to cancer cell CM. Our screening identified CCL8 as the top upregulated soluble factor in Br-TAM. This chemokine is involved in the regulation of activation of immune cells involved in inflammatory responses [27]. CCL8 was recently reported to have a role in metastasis formation in melanoma [28] and more relevantly in a mouse model of breast cancer [29], where CCL8 promoted cancer cell invasion and motility. Interestingly, SIGLEC1 positive macrophages in the mouse intestine produce high levels of CCL8 in response to sterile and non-sterile inflammatory stimuli [30]. CCL8 production was also shown to sustain dextran sulphate sodium colitis and to recruit pro-inflammatory monocytes to the inflamed site. We demonstrated that TAMs are the major source of CCL8, and CCL8 and SIGLEC1 engage in a tumor cell-TAM regulatory loop, involving TNFα that in turn enhances their expression and leads to increased tumor cell motility. Our data showed that cancer cells and TAMs secrete high levels of TNFα that further supports CCL8 production in the tumor microenvironment and that cancer cells respond to the presence of CCL8 by producing significant higher levels of CSF-1, the major survival and proliferation factor for macrophages. The high concentration of CCL8 not only supports the cancer-TAM crosstalk, but also acts as a monocyte chemoattractant. In mouse models of metastatic breast cancer, CCL8 has been shown to recruit Tregs through its receptor CCR5 and in turn inhibition of CCR5 reduced metastasis [31]. Similarly in breast cancer tumors, infiltrating CCR8+Tregs were associated with immunosuppressive functions and high expression of CCR8 correlated with poor clinical outcomes [32]. Therefore, we postulate that CCL8 will also increase monocyte infiltration in the tumor site, resulting in increased numbers of pro-tumoral TAMs (FIG. 17) and an immunosuppressive microenvironment. Consistent with this positive auto-regulatory loop that potentially increases TAM and Treg recruitment and cancer cell malignancy, SIGLEC1 and CCL8 were associated with shorter disease-specific survival and recurrence-free survival in public datasets derived from whole tumor homogenates. Such data reinforce the concept derived from mouse models that TAMs in the tumor microenvironment promote malignancy, and the identification of uniquely expressed genes in human TAMs provides for new therapeutic targets and diagnostic/prognostic markers, as described herein.

References
1. Cassetta, L. and Pollard, J. W. (2018) Targeting macrophages: therapeutic approaches in cancer. Nat Rev Drug Discov.
2. Chávez-Galán, L., Olleros, M. L., Vesin, D., Garcia, I. (2015) Much More than M1 and M2 Macrophages, There are also CD169(+) and TCR(+) Macrophages. Front Immunol 6, 263.
3. Martinez, F. O., Gordon, S., Locati, M., Mantovani, A. (2006) Transcriptional profiling of the human monocyte-to-macrophage differentiation and polarization: new molecules and patterns of gene expression. J Immunol 177, 7303-11.
4. Finak, G., Bertos, N., Pepin, F., Sadekova, S., Souleimanova, M., Zhao, H., Chen, H., Omeroglu, G., Meterissian, S., Omeroglu, A., Hallett, M., Park, M. (2008) Stromal gene expression predicts clinical outcome in breast cancer. Nat Med 14, 518-27.
5. Ojalvo, L. S., King, W., Cox, D., Pollard, J. W. (2009) High-density gene expression analysis of tumor-associated macrophages from mouse mammary tumors. Am J Pathol 174, 1048-64.
6. Karnoub, A. E., Dash, A. B., Vo, A. P., Sullivan, A., Brooks, M. W., Bell, G. W., Richardson, A. L., Polyak, K., Tubo, R., Weinberg, R. A. (2007) Mesenchymal stem cells within tumour stroma promote breast cancer metastasis. Nature 449, 557-63.
7. Cassetta, L., Noy, R., Swierczak, A., Sugano, G., Smith, H., Wiechmann, L., Pollard, J. W. (2016) Isolation of Mouse and Human Tumor-Associated Macrophages. Adv Exp Med Biol 899, 211-29.
8. Curtis, C., Shah, S. P., Chin, S. F., Turashvili, G., Rueda, O. M., Dunning, M. J., Speed, D., Lynch, A. G., Samarajiwa, S., Yuan, Y., Gräf, S., Ha, G., Haffari, G., Bashashati, A., Russell, R., McKinney, S., Langerød, A., Green, A., Provenzano, E., Wishart, G., Pinder, S., Watson, P., Markowetz, F., Murphy, L., Ellis, I., Purushotham, A., Børresen-Dale, A. L., Brenton, J. D., Tavarë, S., Caldas, C., Aparicio, S., Group, M. (2012) The genomic and transcriptomic architecture of 2,000 breast tumours reveals novel subgroups. Nature 486, 346-52.
9. Jorissen, R. N., Gibbs, P., Christie, M., Prakash, S., Lipton, L., Desai, J., Kerr, D., Aaltonen, L. A., Arango, D., Kruhøffer, M., Orntoft, T. F., Andersen, C. L., Gruidl, M., Kamath, V. P., Eschrich, S., Yeatman, T. J., Sieber, O. M. (2009) Metastasis-Associated Gene Expression Changes Predict Poor Outcomes in Patients with Dukes Stage B and C Colorectal Cancer. Clin Cancer Res 15, 7642-7651.
10. Smith, J. J., Deane, N. G., Wu, F., Merchant, N. B., Zhang, B., Jiang, A., Lu, P., Johnson, J. C., Schmidt, C., Bailey, C. E., Eschrich, S., Kis, C., Levy, S., Washington, M. K., Heslin, M. J., Coffey, R. J., Yeatman, T. J., Shyr, Y., Beauchamp, R. D. (2010) Experimentally derived metastasis gene expression profile predicts recurrence and death in patients with colon cancer. Gastroenterology 138, 958-68.
11. Okayama, H., Kohno, T., Ishii, Y., Shimada, Y., Shiraishi, K., Iwakawa, R., Furuta, K., Tsuta, K., Shibata, T., Yamamoto, S., Watanabe, S., Sakamoto, H., Kumamoto, K., Takenoshita, S., Gotoh, N., Mizuno, H., Sarai, A., Kawano, S., Yamaguchi, R., Miyano, S., Yokota, J. (2012) Identification of genes upregulated in ALK-positive and EGFR/KRAS/ALK-negative lung adenocarcinomas. Cancer Res 72, 100-11.
12. Tsujikawa, T., Kumar, S., Borkar, R. N., Azimi, V., Thibault, G., Chang, Y. H., Balter, A., Kawashima, R., Choe, G., Sauer, D., El Rassi, E., Clayburgh, D. R., Kulesz-Martin, M. F., Lutz, E. R., Zheng, L., Jaffee, E. M., Leyshock, P., Margolin, A. A., Mori, M., Gray, J. W., Flint, P. W., Coussens, L. M. (2017) Quantitative Multiplex Immunohistochemistry Reveals Myeloid-Inflamed Tumor-Immune Complexity Associated with Poor Prognosis. Cell Rep 19, 203-217.
13. Cassetta, L., Kajaste-Rudnitski, A., Coradin, T., Saba, E., Della Chiara, G., Barbagallo, M., Graziano, F., Alfano, M., Cassol, E., Vicenzi, E., Poli, G. (2013) M1 polarization of human monocyte-derived macrophages restricts pre and postintegration steps of HIV-1 replication. AIDS 27, 1847-56.
14. Kitamura, T., Qian, B. Z., Soong, D., Cassetta, L., Noy, R., Sugano, G., Kato, Y., Li, J., Pollard, J. W. (2015) CCL2-induced chemokine cascade promotes breast cancer metastasis by enhancing retention of metastasis-associated macrophages. J Exp Med 212, 1043-59.
15. Yang, C. T., Ma, R., Axton, R. A., Jackson, M., Taylor, A. H., Fidanza, A., Marenah, L., Frayne, J., Mountford, J. C., Forrester, L. M. (2017) Activation of KLF1 Enhances the Differentiation and Maturation of Red Blood Cells from Human Pluripotent Stem Cells. Stem Cells 35, 886-897.
16. Töth, Z. E. and Mezey, E. (2007) Simultaneous visualization of multiple antigens with tyramide signal amplification using antibodies from the same species. J Histochem Cytochem 55, 545-54.
17. Liang, C. C., Park, A. Y., Guan, J. L. (2007) In vitro scratch assay: a convenient and inexpensive method for analysis of cell migration in vitro. Nat Protoc 2, 329-33.
18. Ojalvo, L. S., Whittaker, C. A., Condeelis, J. S., Pollard, J. W. (2010) Gene expression analysis of macrophages that facilitate tumor invasion supports a role for Wnt-signaling in mediating their activity in primary mammary tumors. J Immunol 184, 702-12.
19. Makita, N., Hizukuri, Y., Yamashiro, K., Murakawa, M., Hayashi, Y. (2015) IL-10 enhances the phenotype of M2 macrophages induced by IL-4 and confers the ability to increase eosinophil migration. Int Immunol 27, 131-41.
20. Roomi, M. W., Monterrey, J. C., Kalinovsky, T., Rath, M., Niedzwiecki, A. (2009) Patterns of MMP-2 and MMP-9 expression in human cancer cell lines. Oncol Rep 21, 1323-33.
21. Dominic, P., Ajit, N., Tom, F., Andrew, S. (2018) Continuous Biomarker Assessment by Exhaustive Survival Analysis. BioRxiv.
22. Arwert, E. N., Harney, A. S., Entenberg, D., Wang, Y., Sahai, E., Pollard, J. W., Condeelis, J. S. (2018) A Unidirectional Transition from Migratory to Perivascular Macrophage Is Required for Tumor Cell Intravasation. Cell Rep 23, 1239-1248.
23. Xue, J., Schmidt, S. V., Sander, J., Draffehn, A., Krebs, W., Quester, I., De Nardo, D., Gohel, T. D., Emde, M., Schmidleithner, L., Ganesan, H., Nino-Castro, A., Mallmann, M. R., Labzin, L., Theis, H., Kraut, M., Beyer, M., Latz, E., Freeman, T. C., Ulas, T., Schultze, J. L. (2014) Transcriptome-based network analysis reveals a spectrum model of human macrophage activation. Immunity 40, 274-88.
24. Franklin, R. A., Liao, W., Sarkar, A., Kim, M. V., Bivona, M. R., Liu, K., Pamer, E. G., Li, M. O. (2014) The cellular and molecular origin of tumor-associated macrophages. Science 344, 921-5.
25. Li, C., Luo, X., Lin, Y., Tang, X., Ling, L., Wang, L., Jiang, Y. (2015) A Higher Frequency of CD14+ CD169+ Monocytes/Macrophages in Patients with Colorectal Cancer. PLoS One 10, e0141817.
26. Zhang, Y., Li, J. Q., Jiang, Z. Z., Li, L., Wu, Y., Zheng, L. (2016) CD169 identifies an anti-tumour macrophage subpopulation in human hepatocellular carcinoma. J Pathol 239, 231-41.
27. Proost, P., Wuyts, A., Van Damme, J. (1996) Human monocyte chemotactic proteins-2 and -3: structural and functional comparison with MCP-1. J Leukoc Biol 59, 67-74.
28. Barbai, T., Fejős, Z., Puskas, L. G., Timär, J., Räsö, E. (2015) The importance of microenvironment: the role of CCL8 in metastasis formation of melanoma. Oncotarget 6, 29111-28.
29. Farmaki, E., Chatzistamou, I., Kaza, V., Kiaris, H. (2016) A CCL8 gradient drives breast cancer cell dissemination. Oncogene.
30. Asano, K., Takahashi, N., Ushiki, M., Monya, M., Aihara, F., Kuboki, E., Moriyama, S., lida, M., Kitamura, H., Qiu, C. H., Watanabe, T., Tanaka, M. (2015) Intestinal CD169(+) macrophages initiate mucosal inflammation by secreting CCL8 that recruits inflammatory monocytes. Nat Commun 6, 7802.
31. Halvorsen, E. C., Hamilton, M. J., Young, A., Wadsworth, B. J., LePard, N. E., Lee, H. N., Firmino, N., Collier, J. L., Bennewith, K. L. (2016) Maraviroc decreases CCL8-mediated migration of CCR5(+) regulatory T cells and reduces metastatic tumor growth in the lungs. Oncoimmunology 5, e1150398.
32. Plitas, G., Konopacki, C., Wu, K., Bos, P. D., Morrow, M., Putintseva, E. V., Chudakov, D. M., Rudensky, A. Y. (2016) Regulatory T Cells Exhibit Distinct Features in Human Breast Cancer. Immunity 45, 1122-1134.

1. A method of diagnosing and/or prognosing cancer, predicting efficacy of treatment for cancer, assessing outcome of treatment for cancer, assessing likelihood of metastasis and/or assessing recurrence of cancer, the method comprising:
a) analyzing a biological sample obtained from a subject to determine the presence of one or more target molecules representative of expression of SIGLEC1 and/or CCL8; and
b) comparing the expression level of SIGLEC1 and/or CCL8 determined in (a) with one or more reference values, wherein whether there is a difference in the expression of SIGLEC1 and/or CCL8 in the sample from the subject or not compared to the one or more reference values is indicative of a clinical indication.

2. A method according to claim 1 comprising analyzing a biological sample obtained from a subject to determine the presence of one or more target molecules representative of expression of SIGLEC1.

3. A method according to claim 1 or claim 2 wherein said clinical indication comprises one or more of: the presence or absence of cancer in the subject, likelihood of metastasis, likely outcome of treatment of the cancer in the subject, likelihood of recurrence of the cancer following treatment, an indication of whether the prognosis for the cancer treatment and subject is good or poor, predicted survival (life expectancy) of the subject, and likelihood of benign tissues progressing to malignancy.

4. A method according to any of claims 1 to 3 wherein step a) comprises analyzing a biological sample obtained from a subject to determine the presence of target molecules representative of expression of SIGLEC1, and one or more of CCL8, CD163, and CD68; and and step b) comprises comparing the expression levels of SIGLEC1, and one or more of CCL8, CD163, and CD68, determined in (a) with one or more reference values, wherein whether there is a difference in the expression of SIGLEC1, CCL8, CD163 and/or CD68 in the sample from the subject compared to the one or more reference values is indicative of a clinical indication.

5. A method according to any of claims 1 to 3 wherein step a) comprises analyzing a biological sample obtained from a subject to determine the presence of target molecules representative of expression of CCL8, and one or more of SIGLEC1, CD163, and CD68; and step b) comprises comparing the expression levels of CCL8, and the one or more of SIGLEC1, CD163, and CD68, determined in (a) with one or more reference values, wherein whether there is a difference in the expression of CCL8, SIGLEC1, CD163 and/or CD68 in the sample from the subject compared to the one or more reference values is indicative of a clinical indication.

6. A method of treating cancer in a subject comprising:
a) analyzing a biological sample obtained from a subject to determine the presence of one or more target molecules representative of expression of SIGLEC1 and/or CCL8; and
b) comparing the expression level of SIGLEC1 and/or CCL8 determined in (a) with one or more reference values, and providing the subject with a particular treatment for cancer or not according to whether there is a difference in the expression of SIGLEC1 in the sample from the subject or not compared to the one or more reference values.

7. A method of treatment according to claim 6 comprising determining the expression levels of two or more of SIGLEC1, CCL8,CD163, and CD68, and comparing those expression levels with reference values.

8. A method according to any preceding claim comprising determining the expression levels of:
i) SIGLEC1, CCL8, and CD163; or
ii) SIGLEC1, CCL8, and CD68; or
iii) SIGLEC1, CCL8, CD163 and CD68.

9. A method according to any preceding claim wherein the biological sample comprises, or substantially consists of, stromal tissue that is or was adjacent to suspected cancerous cells.

10. A method according to claim 9 wherein the stromal tissue was adjacent cancerous cells and treatment was carried out to remove or destroy the cancerous cells, such that the method comprises an analysis of how effective that treatment was.

11. A method according to claim 10 wherein the stromal tissue was removed weeks or months after the treatment to remove or destroy the cancerous cells.

12. A kit comprising binding partners capable of binding to target molecules representative of expression of SIGLEC1 and/or CCL8, and optionally CD163 and/or CD68.

13. A kit according to claim 12 further comprising indicators capable of indicating when said binding occurs.

14. An assay device comprising:
a) a loading area for receipt of a biological sample;
b) binding partners specific for target molecules representative of expression of SIGLEC1 and/or CCL8, and optionally CD163 and/or CD68; and
c) detection means to detect the levels of said target molecules present in the sample.

15. An assay device according to claim 14 comprising:
a) a loading area for receipt of a biological sample;
b) binding partners specific for target molecules representative of expression of CCL8, and optionally SIGLEC1 and/or CD163 and/or CD68; and
c) detection means to detect the levels of said target molecules present in the sample.

16. A method of identifying one or more molecules for use in treating cancer comprising:
a) preparing a candidate molecule;
b) contacting a cell that expresses SIGLEC1, CD163, CCL8, and/or a CCL8 receptor, with the candidate molecule; and
c) determining whether said candidate molecule binds the SIGLEC1, CD163, CCL8 and/or CCL8 receptor and affects its activity.

17. A method of identifying one or more molecules for use in treating cancer comprising:
a) preparing a candidate molecule;
b) contacting a cell that expresses SIGLEC1, CD163, CCL8, and/or a CCL8 receptor, with the candidate molecule; and
c) determining whether said candidate molecule interferes with either transcription or translation of the SIGLEC1, CD163, CCL8 and/or CCL8 receptor and thereby affects its expression.

18. A method according to claim 16 or claim 17 wherein the cell is either an induced Pluripotent stem cell (iPS) derived macrophage conditioned by tumor cell conditioned media or a cell from a mouse model of cancer.

19. A method according to any of claims 16 to 18 wherein the CCL8 receptor is CCR1, CCR2, CCR5 or CCR8.


SEQUENCE LISTING (14 sequences)

SEQ ID NO: 1 - DNA, 50 bp, Artificial Sequence
  Probe Sequence (Illumina Human HT 12 V4 probes) for SIGLEC1
gagaccacgcagctcattgatcctgatgcagccacatgtgagacctcaac

SEQ ID NO: 2 - DNA, 50 bp, Artificial Sequence
  Probe Sequence (Illumina Human HT 12 V4 probes) for CD163
agccacaacaggtcgctcatcccgtcagtcatcctttattgcagtcggga

SEQ ID NO: 3 - DNA, 50 bp, Artificial Sequence
  Probe Sequence (Illumina Human HT 12 V4 probes) for CCL8
gtcattgttctccctcctacctgtctgtagtgttgtggggtcctcccatg

SEQ ID NO: 4 - DNA, 21 bp, Artificial Sequence
  forward qPCR primer for GAPDH
ggagcgagatccctccaaaat

SEQ ID NO: 5 - DNA, 23 bp, Artificial Sequence
  reverse qPCR primer for GAPDH
ggctgttgtcatacttctcatgg

SEQ ID NO: 6 - DNA, 23 bp, Artificial Sequence
  forward qPCR primer for CX3CR1
actttgagtacgatgatttggct

SEQ ID NO: 7 - DNA, 22 bp, Artificial Sequence
  reverse qPCR primer for CX3CR1
ggtaaatgtcggtgacactctt

SEQ ID NO: 8 - DNA, 20 bp, Artificial Sequence
  forward qPCR primer for SIGLEC1
cctcggggaggaacatcctt

SEQ ID NO: 9 - DNA, 19 bp, Artificial Sequence
  reverse qPCR primer for SIGLEC1
aggcgtaccccatccttga

SEQ ID NO: 10 - DNA, 21 bp, Artificial Sequence
  forward qPCR primer for CCR2
tacggtgctccctgtcataaa

SEQ ID NO: 11 - DNA, 21 bp, Artificial Sequence
  reverse qPCR primer for CCR2
taagatgaggacgaccagcat

SEQ ID NO: 12 - DNA, 23 bp, Artificial Sequence
  forward qPCR primer for CCL8
tggagagctacacaagaatcacc

SEQ ID NO: 13 - DNA, 21 bp, Artificial Sequence
  reverse qPCR primer for CCL8
tggtccagatgcttcatggaa

SEQ ID NO: 14 - DNA, 50 bp, Artificial Sequence
  Probe Sequence (Illumina Human HT 12 V4 probes) for CD68
gacggggttttccttgctcctgccaggattaaaagtccatgagtttcttg